(19) United States
(12) Patent Application Publication
(10) Pub. No.: US 2014/0080188 A1
(43) Pub. Date: Mar. 20, 2014

(54) YEAST MICROORGANISMS WITH REDUCED 2,3-BUTANEDIOL ACCUMULATION FOR IMPROVED PRODUCTION OF FUELS, CHEMICALS, AND AMINO ACIDS

(71) Applicant: GEVO, Inc., Englewood, CO (US)

(72) Inventors: Christopher Smith, Parker, CO (US); Thomas Buelter, Santa Monica, CA (US); Peter Meinhold, Topanga, CA (US); Aristos Aristidou, Maple Grove, MN (US); Stephanie Porter-Scheinman, Conifer, CO (US)

(73) Assignee: GEVO, Inc., Englewwod, CO (US)

(21) Appl. No.: 14/003,534

(22) PCT Filed: Mar. 9, 2012
(86) PCT No.: PCT/US12/28460

§ 371 Date: Nov. 14, 2013

Related U.S. Application Data
(60) Provisional application No. 61/451,042, filed on Mar. 9, 2011.

(51) Int. Cl. C12P 7/16; C12N 15/81

(52) U.S. Cl. CPC C12P 7/16; C12N 15/81
USPC 435/160; 435/254.2; 435/254.21

(57) ABSTRACT

The present invention relates to recombinant microorganisms comprising biosynthetic pathways and methods of using said recombinant microorganisms to produce various beneficial metabolites. In various aspects of the invention, the recombinant microorganisms may further comprise one or more modifications resulting in the reduction or elimination of an acetolactate-derived by-product such as 2,3-butanediol. In various embodiments described herein, the recombinant microorganisms may be microorganisms of the Saccharomyces clade, Crabtree-negative yeast microorganisms, Crabtree-positive yeast microorganisms, post-WGD (whole genome duplication) yeast microorganisms, pre-WGD (whole genome duplication) yeast microorganisms, and non-fermenting yeast microorganisms.

### CROSS REFERENCE TO RELATED APPLICATIONS

This application claims priority to U.S. Provisional Application Ser. No. 61/451,042, filed Mar. 9, 2011, which is hereby incorporated by reference in its entirety for all purposes.

### ACKNOWLEDGMENT OF GOVERNMENTAL SUPPORT

This invention was made with government support under Contract No. 2009-10006-05919, awarded by the United States Department of Agriculture. The government has certain rights in the invention.

### TECHNICAL FIELD

Recombinant microorganisms and methods of producing such microorganisms are provided. Also provided are methods of producing beneficial metabolites including fuels, chemicals, and amino acids by contacting a suitable substrate with said recombinant microorganisms and enzymatic preparations therefrom.

### BACKGROUND

The ability of microorganisms to convert sugars to beneficial metabolites including fuels, chemicals, and amino acids has been widely described in the literature in recent years. See, e.g., Alper et al., 2009, Nature Microbiol. Rev. 7: 715-723 and McCourt et al., 2006, Amino Acids 31: 173-210. Recombinant engineering techniques have enabled the creation of microorganisms that express biosynthetic pathways capable of producing a number of useful products, such as valine, isoleucine, leucine, and pantothenic acid (vitamin B5). In addition, fuels such as isobutanol have been produced recombinantly in microorganisms expressing a heterologous metabolic pathway (See, e.g., WO/2007/050671 to Donaldson et al., and WO/2008/098227 to Liao et al.). Although engineered microorganisms represent potentially useful tools for the renewable production of fuels, chemicals, and amino acids, many of these microorganisms have fallen short of commercial relevance due to their low performance characteristics, including low productivity, low titers, and low yields.

One of the primary reasons for the sub-optimal performance observed in many existing microorganisms is the undesirable conversion of pathway intermediates to unwanted by-products. The present inventors have identified various unwanted by-products, including acetoin and 2,3-butanediol, which are derived from acetolactate, an intermediate of many biosynthetic pathways used to produce fuels, chemicals, and amino acids. Until now, the enzymatic activities responsible for the production of these unwanted by-products had not fully been characterized. The present application shows that the enzymatic activities of endogenous yeast enzymes, including, but not limited to, acetolactate decarboxylases, diacetyl reductases, and/or acetoin reductases contribute to the formation of 2,3-butanediol from acetolactate. Further, certain enzymes responsible for the accumulation of 2,3-butanediol can compete with the engineered biosynthetic pathways for reduced co-factors, NADH and/or NADPH, thereby reducing productivity and/or yield of the desired metabolite.

The present invention results from the study of the enzymes involved in the conversion of acetolactate to 2,3-butanediol and shows that the suppression of one or more of these enzymes considerably reduces or eliminates the formation of 2,3-butanediol.

### SUMMARY OF THE INVENTION

The present inventors have discovered that unwanted by-products such as diacetyl, acetoin and 2,3-butanediol can accumulate during various fermentation processes, including fermentation of the biofuel candidate, isobutanol. The accumulation of these unwanted by-products results from the undesirable conversion of acetolactate, a key intermediate in certain biosynthetic pathways. The enzymatic conversion of acetolactate to these unwanted by-products can hinder the optimal yield of a desirable acetolactate-derived product by diverting carbon flow and by competing for reduced co-factors. Furthermore, the accumulation of unwanted by-products such as 2,3-butanediol may inhibit downstream processing of the desired metabolite and complicate purification techniques. Therefore, the present inventors have developed methods for reducing the conversion of acetolactate to acetoin and/or 2,3-butanediol during processes where acetolactate acts as a pathway intermediate.

In a first aspect, the present invention relates to a recombinant microorganism comprising a biosynthetic pathway of which acetolactate is an intermediate, wherein said recombinant microorganism is (a) substantially free of an enzyme catalyzing a pathway step in the conversion of acetolactate to 2,3-butanediol; and/or (b) engineered to reduce or eliminate the expression or activity of an enzyme catalyzing a pathway step the conversion of acetolactate to 2,3-butanediol.

In one embodiment, the invention is directed to a recombinant microorganism comprising a biosynthetic pathway which uses acetolactate as an intermediate, wherein said recombinant microorganism is engineered to reduce or eliminate the expression or activity of one or more enzymes catalyzing the conversion of acetolactate to acetoin. In some embodiments, the enzyme catalyzing the conversion of acetolactate to acetoin is an acetolactate decarboxylase (ALDC). In an exemplary embodiment, the acetolactate decarboxylase (ALDC) is the S. cerevisiae acetolactate decarboxylase or a homolog or variant thereof.

In one embodiment, the invention is directed to a recombinant microorganism comprising a biosynthetic pathway which uses acetolactate as an intermediate, wherein said recombinant microorganism is engineered to reduce or eliminate the expression or activity of one or more enzymes catalyzing the conversion of diacetyl to acetoin. In some embodiments, the enzyme catalyzing the conversion of diacetyl to acetoin is a diacetyl reductase. In an exemplary embodiment, the diacetyl reductase is the S. cerevisiae protein, Oye2p (SEQ ID NO: 2), or a homolog or variant thereof. In another exemplary embodiment, the diacetyl reductase is the S. cerevisiae protein, Ara1p (SEQ ID NO: 4), or a homolog or variant thereof. In yet another exemplary embodiment, the diacetyl reductase is selected from the S. cerevisiae proteins Bdh1p (SEQ ID NO: 6), Bdh2p (SEQ ID NO: 8), Erg19p (SEQ ID NO: 10), Gcy1p (SEQ ID NO: 12), Gre3p (SEQ ID NO: 14), Oye3p (SEQ ID NO: 16), Trr1p (SEQ ID NO: 18), Ypr1p (SEQ ID NO: 20), Zwf1p (SEQ ID NO: 22), and YPL088W (SEQ ID NO: 24), or homologs or variants thereof.

In one embodiment, the invention is directed to a recombinant microorganism comprising a biosynthetic pathway which uses acetolactate as an intermediate, wherein said recombinant microorganism is engineered to reduce or eliminate the expression or activity of one or more enzymes catalyzing the conversion of acetoin to 2,3-butanediol. In some embodiments, the enzyme catalyzing the conversion of acetoin to 2,3-butanediol is an acetoin reductase. In an exemplary embodiment, the acetoin reductase is the S. cerevisiae protein, Bdh1p, or a homolog or variant thereof. In another exemplary embodiment, acetoin reductase is the S. cerevisiae protein, Bdh2p, or a homolog or variant thereof. In yet another exemplary embodiment, the acetoin reductase is the S. cerevisiae protein, Ara1p, or a homolog or variant thereof. In yet another exemplary embodiment, the acetoin reductase is selected from the S. cerevisiae proteins Erg19p, Gcy1p, Gre3p, Oye2p, Oye3p, Trr1p, Ypr1p, Zwf1p, and YPL088W, or homologs or variants thereof.

In one embodiment, the invention is directed to a recombinant microorganism comprising a biosynthetic pathway which uses acetolactate as an intermediate, wherein the recombinant microorganism is engineered to reduce or eliminate the expression or activity of at least two of the following: (i) one or more enzymes catalyzing the conversion of acetolactate to acetoin; (ii) one or more enzymes catalyzing the conversion of diacetyl to acetoin; and/or (iii) one or more enzymes catalyzing the conversion of acetoin to 2,3-butanediol. In another embodiment, the invention is directed to a recombinant microorganism comprising a biosynthetic pathway which uses acetolactate as an intermediate, wherein the recombinant microorganism is engineered to reduce or eliminate the expression or activity of all of the following: (i) one or more enzymes catalyzing the conversion of acetolactate to acetoin; (ii) one or more enzymes catalyzing the conversion of diacetyl to acetoin; and (iii) one or more enzymes catalyzing the conversion of acetoin to 2,3-butanediol.

In yet another embodiment, the recombinant microorganism comprising a biosynthetic pathway which uses acetolactate as an intermediate may be engineered to reduce the activity or expression of an endogenous transporter protein selected from the group consisting of Opt1p, Opt2p, YGL141W, Adp1p, Arb1p, Atm1p, Aus1p, Bpt1p, Mdl1p, Mdl2p, Nft1p, Pdr5p, Pdr10p, Pdr11p, Pdr12p, Pdr15p, Pdr18p, Pxa1p, Pxa2p, Rli1p, Snq2p, Step 6p, Vma8p, Vmr1p, Ybt1p, Ycf1p, Yor1p, YKR104W, YOL075C, Aqr1p, Atr1p, Azr1p, Dtr1p, Enb1p, Flr1p, Hol1p, Pdr8p, Qdr1p, Qdr2p, Qdr3p, Seo1p, Sge1p, Ssu1p, Thi7p, Tpn1p, Vba5p, YIL166C, Agp1p, Agp2p, Agp3p, Alp1p, Bap2p, Bap3p, Bio5p, Can1p, Dip5p, Gap1p, Gnp1p, Hip1p, Hnm1p, Lyp1p, Mmp1p, Put4p, Sam3p, Ssy1p, Tat1p, Tat2p, Tpo1p, Tpo2p, Tpo3p, Tpo4p, Tpo5p, and Uga4p. In a further embodiment, the recombinant microorganism comprising a biosynthetic pathway which uses acetolactate as an intermediate may be engineered to reduce the activity or expression of an endogenous transcriptional regulator of an endogenous transporter protein. In an exemplary embodiment, the transcriptional regulator is War1p.

In various embodiments described herein, the recombinant microorganism may comprise a biosynthetic pathway which uses acetolactate as an intermediate. The biosynthetic pathway which uses acetolactate as an intermediate may be selected from a pathway for the biosynthesis of isobutanol, 1-butanol, valine, leucine, pantothenic acid, isobutylene, 3-methyl-1-butanol, 4-methyl-1-pentanol, and coenzyme A.

In various embodiments described herein, the recombinant microorganisms of the application that comprise a biosynthetic pathway of which acetolactate is an intermediate may be further engineered to reduce or eliminate the expression and/or activity of one or more enzymes selected from a pyruvate decarboxylase (PDC), a glycerol-3-phosphate dehydrogenase (GPD), a 3-keto acid reductase (3-KAR), or an aldehyde dehydrogenase (ALDH).

In one embodiment, the invention is directed to a recombinant microorganism for producing isobutanol, wherein said recombinant microorganism comprises an isobutanol producing metabolic pathway and wherein said microorganism is engineered to reduce or eliminate the expression or activity of one or more enzymes catalyzing the conversion of acetolactate to acetoin. In some embodiments, the enzyme catalyzing the conversion of acetolactate to acetoin is an acetolactate decarboxylase (ALDC). In an exemplary embodiment, the acetolactate decarboxylase is the S. cerevisiae acetolactate decarboxylase or a homolog or variant thereof.

In another embodiment, the invention is directed to a recombinant microorganism for producing isobutanol, wherein said recombinant microorganism comprises an isobutanol producing metabolic pathway and wherein said microorganism is engineered to reduce or eliminate the expression or activity of one or more enzymes catalyzing the conversion of diacetyl to acetoin. In some embodiments, the enzyme catalyzing the conversion of diacetyl to acetoin is a diacetyl reductase. In an exemplary embodiment, the diacetyl reductase is the S. cerevisiae protein, Oye2p, or a homolog or variant thereof. In another exemplary embodiment, the diacetyl reductase is the S. cerevisiae protein, Ara1p, or a homolog or variant thereof. In yet another exemplary embodiment, the diacetyl reductase is selected from the S. cerevisiae proteins Bdh1p, Bdh2p, Erg19p, Gcy1p, Gre3p, Oye3p, Trr1p, Ypr1p, Zwf1p, and YPL088W, or homologs or variants thereof.

In yet another embodiment, the invention is directed to a recombinant microorganism for producing isobutanol, wherein said recombinant microorganism comprises an isobutanol producing metabolic pathway and wherein said microorganism is engineered to reduce or eliminate the expression or activity of one or more enzymes catalyzing the conversion of acetoin to 2,3-butanediol. In some embodiments, the enzyme catalyzing the conversion of acetoin to 2,3-butanediol is an acetoin reductase. In an exemplary embodiment, the acetoin reductase is the S. cerevisiae protein, Bdh1p, or a homolog or variant thereof. In another exemplary embodiment, acetoin reductase is the S. cerevisiae protein, Bdh2p, or a homolog or variant thereof. In yet another exemplary embodiment, the acetoin reductase is the S. cerevisiae protein, Ara1p, or a homolog or variant thereof. In yet another exemplary embodiment, the acetoin reductase is selected from the S. cerevisiae proteins Erg19p, Gcy1p, Gre3p, Oye2p, Oye3p, Trr1p, Ypr1p, Zwf1p, and YPL088W, or homologs or variants thereof.

In one embodiment, the invention is directed to a recombinant microorganism for producing isobutanol, wherein said recombinant microorganism comprises an isobutanol producing metabolic pathway and wherein the recombinant microorganism is engineered to reduce or eliminate the expression or activity of at least two of the following: (i) one or more enzymes catalyzing the conversion of acetolactate to acetoin; (ii) one or more enzymes catalyzing the conversion of diacetyl to acetoin; and/or (iii) one or more enzymes catalyzing the conversion of acetoin to 2,3-butanediol. In another embodiment, the invention is directed to a recombinant microorganism comprising a biosynthetic pathway which uses acetolactate as an intermediate, wherein the recombinant microorganism is engineered to reduce or eliminate the expression or activity of all of the following: (i) one or more enzymes catalyzing the conversion of acetolactate to acetoin; (ii) one or more enzymes catalyzing the conversion of diacetyl to acetoin; and (iii) one or more enzymes catalyzing the conversion of acetoin to 2,3-butanediol.

In yet another embodiment, the recombinant microorganism comprising an isobutanol producing metabolic pathway may be engineered to reduce the activity or expression of an endogenous transporter protein selected from the group consisting of Opt1p, Opt2p, YGL141W, Adp1p, Arb1p, Atm1p, Aus1p, Bpt1p, Mdl1p, Mdl2p, Nft1p, Pdr5p, Pdr10p, Pdr11p, Pdr12p, Pdr15p, Pdr18p, Pxa1p, Pxa2p, Rli1p, Snq2p, Step 6p, Vma8p, Vmr1p, Ybt1p, Ycf1p, Yor1p, YKR104W, YOL075C, Aqr1p, Atr1p, Azr1p, Dtr1p, Enb1p, Flr1p, Hol1p, Pdr8p, Qdr1p, Qdr2p, Qdr3p, Seo1p, Sge1p, Ssu1p, Thi7p, Tpn1p, Vba5p, YIL166C, Agp1p, Agp2p, Agp3p, Alp1p, Bap2p, Bap3p, Bio5p, Can1p, Dip5p, Gap1p, Gnp1p, Hip1p, Hnm1p, Lyp1p, Mmp1p, Put4p, Sam3p, Ssy1p, Tat1p, Tat2p, Tpo1p, Tpo2p, Tpo3p, Tpo4p, Tpo5p, and Uga4p. In a further embodiment, the recombinant microorganism comprising an isobutanol producing metabolic pathway may be engineered to reduce the activity or expression of an endogenous transcriptional regulator of an endogenous transporter protein. In an exemplary embodiment, the transcriptional regulator is War1p.

In one embodiment, the isobutanol producing metabolic pathway comprises at least one exogenous gene encoding a polypeptide that catalyzes a step in the conversion of pyruvate to isobutanol. In another embodiment, the isobutanol producing metabolic pathway comprises at least two exogenous genes encoding polypeptides that catalyze steps in the conversion of pyruvate to isobutanol. In yet another embodiment, the isobutanol producing metabolic pathway comprises at least three exogenous genes encoding polypeptides that catalyze steps in the conversion of pyruvate to isobutanol. In yet another embodiment, the isobutanol producing metabolic pathway comprises at least four exogenous genes encoding polypeptides that catalyze steps in the conversion of pyruvate to isobutanol. In yet another embodiment, the isobutanol producing metabolic pathway comprises at least five exogenous genes encoding polypeptides that catalyze steps in the conversion of pyruvate to isobutanol. In yet another embodiment, all of the isobutanol producing metabolic pathway steps in the conversion of pyruvate to isobutanol are converted by exogenously encoded enzymes.

In one embodiment, one or more of the isobutanol pathway genes encodes an enzyme that is localized to the cytosol. In one embodiment, the recombinant microorganisms comprise an isobutanol producing metabolic pathway with at least one isobutanol pathway enzyme localized in the cytosol. In another embodiment, the recombinant microorganisms comprise an isobutanol producing metabolic pathway with at least two isobutanol pathway enzymes localized in the cytosol. In yet another embodiment, the recombinant microorganisms comprise an isobutanol producing metabolic pathway with at least three isobutanol pathway enzymes localized in the cytosol. In yet another embodiment, the recombinant microorganisms comprise an isobutanol producing metabolic pathway with at least four isobutanol pathway enzymes localized in the cytosol. In an exemplary embodiment, the recombinant microorganisms comprise an isobutanol producing metabolic pathway with five isobutanol pathway enzymes localized in the cytosol. In yet another exemplary embodiment, the recombinant microorganisms comprise an isobutanol producing metabolic pathway with all isobutanol pathway enzymes localized in the cytosol.

In various embodiments described herein, the isobutanol pathway genes may encode enzyme(s) selected from the group consisting of acetolactate synthase (ALS), ketol-acid reductoisomerase (KARI), dihydroxyacid dehydratase (DHAD), 2-keto-acid decarboxylase, e.g., keto-isovalerate decarboxylase (KIVD), and alcohol dehydrogenase (ADH). In one embodiment, the KARI is an NADH-dependent KARI (NKR). In another embodiment, the ADH is an NADH-dependent ADH. In yet another embodiment, the KARI is an NADH-dependent KARI (NKR) and the ADH is an NADH-dependent ADH.

In various embodiments described herein, the recombinant microorganisms of the invention that comprise an isobutanol producing metabolic pathway may be further engineered to reduce or eliminate the expression or activity of one or more enzymes selected from a pyruvate decarboxylase (PDC), a glycerol-3-phosphate dehydrogenase (GPD), a 3-keto acid reductase (3-KAR), or an aldehyde dehydrogenase (ALDH).

As described herein, in preferred embodiments, the recombinant microorganisms of the application are recombinant yeast microorganisms.

In various embodiments described herein, the recombinant microorganisms may be microorganisms of the Saccharomyces clade, Saccharomyces sensu stricto microorganisms, Crabtree-negative yeast microorganisms, Crabtree-positive yeast microorganisms, post-WGD (whole genome duplication) yeast microorganisms, pre-WGD (whole genome duplication) yeast microorganisms, and non-fermenting yeast microorganisms.

In some embodiments, the recombinant yeast microorganisms may be members of the Saccharomyces clade, Saccharomyces sensu stricto microorganisms, Crabtree-negative yeast microorganisms, Crabtree-positive yeast microorganisms, post-WGD (whole genome duplication) yeast microorganisms, pre-WGD (whole genome duplication) yeast microorganisms, and non-fermenting yeast microorganisms.

In some embodiments, the recombinant microorganisms may be yeast recombinant microorganisms of the Saccharomyces clade.

In some embodiments, the recombinant microorganisms may be Saccharomyces sensu stricto microorganisms. In one embodiment, the Saccharomyces sensu stricto is selected from the group consisting of S. cerevisiae, S. kudriavzevii, S. mikatae, S. bayanus, S. uvarum, S. carocanis and hybrids thereof.

In some embodiments, the recombinant microorganisms may be Crabtree-negative recombinant yeast microorganisms. In one embodiment, the Crabtree-negative yeast microorganism is classified into a genera selected from the group consisting of Saccharomyces, Kluyveromyces, Pichia, Issatchenkia, Hansenula, or Candida. In additional embodiments, the Crabtree-negative yeast microorganism is selected from Saccharomyces kluyveri, Kluyveromyces lactis, Kluyveromyces marxianus, Pichia anomala, Pichia stipitis, Hansenula anomala, Candida utilis and Kluyveromyces waltii.

In some embodiments, the recombinant microorganisms may be Crabtree-positive recombinant yeast microorganisms. In one embodiment, the Crabtree-positive yeast microorganism is classified into a genera selected from the group consisting of Saccharomyces, Kluyveromyces, Zygosaccharomyces, Debaryomyces, Candida, Pichia and Schizosaccharomyces. In additional embodiments, the Crabtree-positive yeast microorganism is selected from the group consisting of Saccharomyces cerevisiae, Saccharomyces uvarum, Saccharomyces bayanus, Saccharomyces paradoxus, Saccharomyces castelli, Kluyveromyces thermotolerans, Candida glabrata, Z. bailli, Z. rouxii, Debaryomyces hansenii, Pichia pastorius, Schizosaccharomyces pombe, and Saccharomyces uvarum.

In some embodiments, the recombinant microorganisms may be post-WGD (whole genome duplication) yeast recombinant microorganisms. In one embodiment, the post-WGD yeast recombinant microorganism is classified into a genera selected from the group consisting of Saccharomyces or Candida. In additional embodiments, the post-WGD yeast is selected from the group consisting of Saccharomyces cerevisiae, Saccharomyces uvarum, Saccharomyces bayanus, Saccharomyces paradoxus, Saccharomyces castelli, and Candida glabrata.

In some embodiments, the recombinant microorganisms may be pre-WGD (whole genome duplication) yeast recombinant microorganisms. In one embodiment, the pre-WGD yeast recombinant microorganism is classified into a genera selected from the group consisting of Saccharomyces, Kluyveromyces, Candida, Pichia, Issatchenkia, Debaryomyces, Hansenula, Pachysolen, Yarrowia and Schizosaccharomyces. In additional embodiments, the pre-WGD yeast is selected from the group consisting of Saccharomyces kluyveri, Kluyveromyces thermotolerans, Kluyveromyces marxianus, Kluyveromyces waltii, Kluyveromyces lactis, Candida tropicalis, Pichia pastoris, Pichia anomala, Pichia stipitis, Issatchenkia orientalis, Issatchenkia occidentalis, Debaryomyces hansenii, Hansenula anomala, Pachysolen tannophilis, Yarrowia lipolytica, and Schizosaccharomyces pombe.

In some embodiments, the recombinant microorganisms may be microorganisms that are non-fermenting yeast microorganisms, including, but not limited to those, classified into a genera selected from the group consisting of Tricosporon, Rhodotorula, Myxozyma, or Candida. In a specific embodiment, the non-fermenting yeast is C. xestobii.

In another aspect, the present invention provides methods of producing beneficial metabolites including fuels, chemicals, and amino acids using a recombinant microorganism as described herein. In one embodiment, the method includes cultivating the recombinant microorganism in a culture medium containing a feedstock providing the carbon source until the metabolite is produced and optionally, recovering the metabolite. In one embodiment, the microorganism produces the metabolite from a carbon source at a yield of at least about 5 percent theoretical. In another embodiment, the microorganism produces the metabolite at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent, at least about 85 percent, at least about 90 percent, at least about 95 percent, or at least about 97.5 percent theoretical. In one embodiment, the metabolite may be derived from a biosynthetic pathway which uses acetolactate as an intermediate, including, but not limited to, isobutanol, 2-butanol, 1-butanol, 2-butanone, valine, leucine, pantothenic acid, isobutylene, 3-methyl-1-butanol, 4-methyl-1-pentanol, and coenzyme A biosynthetic pathways. In an exemplary embodiment, the metabolite is isobutanol.

In one embodiment, the recombinant microorganism converts the carbon source to the desired metabolite under aerobic conditions. In another embodiment, the recombinant microorganism converts the carbon source to the desired metabolite under microaerobic conditions. In yet another embodiment, the recombinant microorganism converts the carbon source to the desired metabolite under anaerobic conditions.

### BRIEF DESCRIPTION OF DRAWINGS

Illustrative embodiments of the invention are illustrated in the drawings, in which:

FIG. 1 illustrates an exemplary embodiment of an isobutanol pathway.

FIG. 2 illustrates an exemplary embodiment of an NADH-dependent isobutanol pathway.

FIG. 3 illustrates biosynthetic pathways utilizing acetolactate as an intermediate.

FIG. 4 illustrates the diacetyl, acetoin, and 2,3-butanediol pathways in the context of isobutanol production.

FIG. 5 illustrates the conversion of acetolactate to acetoin via the action of an acetolactate decarboxylase.

FIG. 6 illustrates the conversion of diacetyl to acetoin via the action of a diacetyl reductase.

FIG. 7 illustrates the conversion of acetoin to 2,3-butanediol via the action of an acetoin reductase.

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “genus” is defined as a taxonomic group of related species according to the Taxonomic Outline of Bacteria and Archaea (Garrity, G. M., Lilburn, T. G., Cole, J. R., Harrison, S. H., Euzeby, J., and Tindall, B. J. (2007) The Taxonomic Outline of Bacteria and Archaea. TOBA Release 7.7, March 2007. Michigan State University Board of Trustees.

The term “species” is defined as a collection of closely related organisms with greater than 97% 16S ribosomal RNA sequence homology and greater than 70% genomic hybridization and sufficiently different from all other organisms so as to be recognized as a distinct unit.

The terms “recombinant microorganism,” “modified microorganism,” and “recombinant host cell” are used interchangeably herein and refer to microorganisms that have been genetically modified to express or to overexpress endogenous polynucleotides, to express heterologous polynucleotides, such as those included in a vector, in an integration construct, or which have an alteration in expression of an endogenous gene. By “alteration” it is meant that the expression of the gene, or level of a RNA molecule or equivalent RNA molecules encoding one or more polypeptides or polypeptide subunits, or activity of one or more polypeptides or polypeptide subunits is up regulated or down regulated, such that expression, level, or activity is greater than or less than that observed in the absence of the alteration. For example, the term “alter” can mean “inhibit,” but the use of the word “alter” is not limited to this definition. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence. The level of expression of a desired product in a host cell may be determined on the basis of either the amount of corresponding mRNA that is present in the cell, or the amount of the desired product encoded by the selected sequence. For example, mRNA transcribed from a selected sequence can be quantitated by qRT-PCR or by Northern hybridization (see Sambrook et al., Molecular Cloning: A Laboratory Manual, Cold Spring Harbor Laboratory Press (1989)). Protein encoded by a selected sequence can be quantitated by various methods, e.g., by ELISA, by assaying for the biological activity of the protein, or by employing assays that are independent of such activity, such as western blotting or radioimmunoassay, using antibodies that recognize and bind the protein. See Sambrook et al., 1989, supra.

The term “overexpression” refers to an elevated level (e.g., aberrant level) of mRNAs encoding for a protein(s), and/or to elevated levels of protein(s) in cells as compared to similar corresponding unmodified cells expressing basal levels of mRNAs or having basal levels of proteins. In particular embodiments mRNA(s) or protein(s) may be overexpressed by at least 2-fold, 3-fold, 4-fold, 5-fold, 6-fold, 8-fold, 10-fold, 12-fold, 15-fold or more in microorganisms engineered to exhibit increased gene mRNA, protein, and/or activity.

As used herein and as would be understood by one of ordinary skill in the art, “reduced activity and/or expression” of a protein such as an enzyme can mean either a reduced specific catalytic activity of the protein (e.g. reduced activity) and/or decreased concentrations of the protein in the cell (e.g. reduced expression).

The term “wild-type microorganism” describes a cell that occurs in nature, i.e. a cell that has not been genetically modified. A wild-type microorganism can be genetically modified to express or overexpress a first target enzyme. This microorganism can act as a parental microorganism in the generation of a microorganism modified to express or overexpress a second target enzyme. In turn, the microorganism modified to express or overexpress a first and a second target enzyme can be modified to express or overexpress a third target enzyme.

Accordingly, a “parental microorganism” functions as a reference cell for successive genetic modification events. Each modification event can be accomplished by introducing a nucleic acid molecule in to the reference cell. The introduction facilitates the expression or overexpression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of heterologous polynucleotides encoding a target enzyme in to a parental microorganism.

The term “engineer” refers to any manipulation of a microorganism that results in a detectable change in the microorganism, wherein the manipulation includes but is not limited to inserting a polynucleotide and/or polypeptide heterologous to the microorganism and mutating a polynucleotide and/or polypeptide native to the microorganism.

The term “mutation” as used herein indicates any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, a nonsense mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are identified and/or enriched through artificial selection pressure. In still other embodiments, the mutations in the microorganism genome are the result of genetic engineering.

The term “biosynthetic pathway”, also referred to as “metabolic pathway”, refers to a set of anabolic or catabolic biochemical reactions for converting one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

As used herein, the term “isobutanol producing metabolic pathway” refers to an enzyme pathway which produces isobutanol from pyruvate.

The term “NADH-dependent” as used herein with reference to an enzyme, e.g., KARI and/or ADH, refers to an enzyme that catalyzes the reduction of a substrate coupled to the oxidation of NADH with a catalytic efficiency that is greater than the reduction of the same substrate coupled to the oxidation of NADPH.

The term “exogenous” as used herein with reference to various molecules, e.g., polynucleotides, polypeptides, enzymes, etc., refers to molecules that are not normally or naturally found in and/or produced by a given yeast, bacterium, organism, microorganism, or cell in nature.

On the other hand, the term “endogenous” or “native” as used herein with reference to various molecules, e.g., polynucleotides, polypeptides, enzymes, etc., refers to molecules that are normally or naturally found in and/or produced by a given yeast, bacterium, organism, microorganism, or cell in nature.

The term “heterologous” as used herein in the context of a modified host cell refers to various molecules, e.g., polynucleotides, polypeptides, enzymes, etc., wherein at least one of the following is true: (a) the molecule(s) is/are foreign (“exogenous”) to (i.e., not naturally found in) the host cell; (b) the molecule(s) is/are naturally found in (e.g., is “endogenous to”) a given host microorganism or host cell but is either produced in an unnatural location or in an unnatural amount in the cell; and/or (c) the molecule(s) differ(s) in nucleotide or amino acid sequence from the endogenous nucleotide or amino acid sequence(s) such that the molecule differing in nucleotide or amino acid sequence from the endogenous nucleotide or amino acid as found endogenously is produced in an unnatural (e.g., greater than naturally found) amount in the cell.

The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a biofuel in a fermentation process. However, a feedstock may contain nutrients other than a carbon source.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a recombinant microorganism as described herein.

The term “fermentation” or “fermentation process” is defined as a process in which a microorganism is cultivated in a culture medium containing raw materials, such as feedstock and nutrients, wherein the microorganism converts raw materials, such as a feedstock, into products.

The term “volumetric productivity” or “production rate” is defined as the amount of product formed per volume of medium per unit of time. Volumetric productivity is reported in gram per liter per hour (g/L/h).

The term “specific productivity” or “specific production rate” is defined as the amount of product formed per volume of medium per unit of time per amount of cells. Specific productivity is reported in gram (or milligram) per gram cell dry weight per hour (g/g h).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product. For example, the theoretical yield for one typical conversion of glucose to isobutanol is 0.41 g/g. As such, a yield of isobutanol from glucose of 0.39 g/g would be expressed as 95% of theoretical or 95% theoretical yield.

The term “titer” is defined as the strength of a solution or the concentration of a substance in solution. For example, the titer of a biofuel in a fermentation broth is described as g of biofuel in solution per liter of fermentation broth (g/L).

“Aerobic conditions” are defined as conditions under which the oxygen concentration in the fermentation medium is sufficiently high for an aerobic or facultative anaerobic microorganism to use as a terminal electron acceptor.

In contrast, “anaerobic conditions” are defined as conditions under which the oxygen concentration in the fermentation medium is too low for the microorganism to use as a terminal electron acceptor. Anaerobic conditions may be achieved by sparging a fermentation medium with an inert gas such as nitrogen until oxygen is no longer available to the microorganism as a terminal electron acceptor. Alternatively, anaerobic conditions may be achieved by the microorganism consuming the available oxygen of the fermentation until oxygen is unavailable to the microorganism as a terminal electron acceptor. Methods for the production of isobutanol under anaerobic conditions are described in commonly owned and co-pending publication, US 2010/0143997, the disclosures of which are herein incorporated by reference in its entirety for all purposes.

“Aerobic metabolism” refers to a biochemical process in which oxygen is used as a terminal electron acceptor to make energy, typically in the form of ATP, from carbohydrates. Aerobic metabolism occurs e.g. via glycolysis and the TCA cycle, wherein a single glucose molecule is metabolized completely into carbon dioxide in the presence of oxygen.

In contrast, “anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a “fermentative pathway.”

In “fermentative pathways”, NAD(P)H donates its electrons to a molecule produced by the same metabolic pathway that produced the electrons carried in NAD(P)H. For example, in one of the fermentative pathways of certain yeast strains, NAD(P)H generated through glycolysis transfers its electrons to pyruvate, yielding ethanol. Fermentative pathways are usually active under anaerobic conditions but may also occur under aerobic conditions, under conditions where NADH is not fully oxidized via the respiratory chain. For example, above certain glucose concentrations, Crabtree positive yeasts produce large amounts of ethanol under aerobic conditions.

The term “byproduct” or “by-product” means an undesired product related to the production of an amino acid, amino acid precursor, chemical, chemical precursor, biofuel, or biofuel precursor.

The term “substantially free” when used in reference to the presence or absence of enzymatic activities (acetolactate decarboxylase, diacetyl reductase, acetoin reductase, 3-KAR, ALDH, PDC, GPD, etc.) in carbon pathways that compete with the desired metabolic pathway (e.g., an isobutanol-producing metabolic pathway) means the level of the protein is substantially less than that of the same protein in the wild-type host, wherein less than about 50% of the wild-type level is preferred and less than about 30% is more preferred. The activity may be less than about 20%, less than about 10%, less than about 5%, or less than about 1% of wild-type activity. Microorganisms which are “substantially free” of a particular enzymatic activity (acetolactate decarboxylase, diacetyl reductase, acetoin reductase, 3-KAR, ALDH, PDC, GPD, etc.) may be created through recombinant means or identified in nature.

The term “non-fermenting yeast” is a yeast species that fails to demonstrate an anaerobic metabolism in which the electrons from NADH are utilized to generate a reduced product via a fermentative pathway such as the production of ethanol and CO₂ from glucose. Non-fermentative yeast can be identified by the “Durham Tube Test” (J. A. Barnett, R. W. Payne, and D. Yarrow. 2000. Yeasts Characteristics and Identification. 3^{rd} edition. p. 28-29. Cambridge University Press, Cambridge, UK) or by monitoring the production of fermentation productions such as ethanol and CO₂.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, DNA, RNA, analogs and fragments thereof. A polynucleotide of three or more nucleotides is also called nucleotidic oligomer or oligonucleotide.

It is understood that the polynucleotides described herein include “genes” and that the nucleic acid molecules described herein include “vectors” or “plasmids.” Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “operon” refers to two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including chemical transformation (e.g. lithium acetate transformation), electroporation, microinjection, biolistics (or particle bombardment-mediated delivery), or agrobacterium mediated transformation.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “protein,” “peptide,” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide.

The term “homolog,” used with respect to an original polynucleotide or polypeptide of a first family or species, refers to distinct polynucleotides or polypeptides of a second family or species which are determined by functional, structural or genomic analyses to be a polynucleotide or polypeptide of the second family or species which corresponds to the original polynucleotide or polypeptide of the first family or species. Most often, homologs will have functional, structural or genomic similarities. Techniques are known by which homologs of a polynucleotide or polypeptide can readily be cloned using genetic probes and PCR. Identity of cloned sequences as homolog can be confirmed using functional assays and/or by genomic mapping of the genes.

A polypeptide has “homology” or is “homologous” to a second polypeptide if the amino acid sequence encoded by a gene has a similar amino acid sequence to that of the second gene. Alternatively, a polypeptide has homology to a second polypeptide if the two polypeptides have “similar” amino acid sequences. (Thus, the terms “homologous polypeptides” or “homologous proteins” are defined to mean that the two polypeptides have similar amino acid sequences).

The term “analog” or “analogous” refers to polynucleotide or polypeptide sequences that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

Recombinant Microorganisms with Reduced By-Product Accumulation

Yeast cells convert sugars to produce pyruvate, which is then utilized in a number of pathways of cellular metabolism. In recent years, yeast cells have been engineered to produce a number of desirable products via pyruvate-driven biosynthetic pathways. In many of these biosynthetic pathways, the initial pathway step is the conversion of endogenous pyruvate to acetolactate.

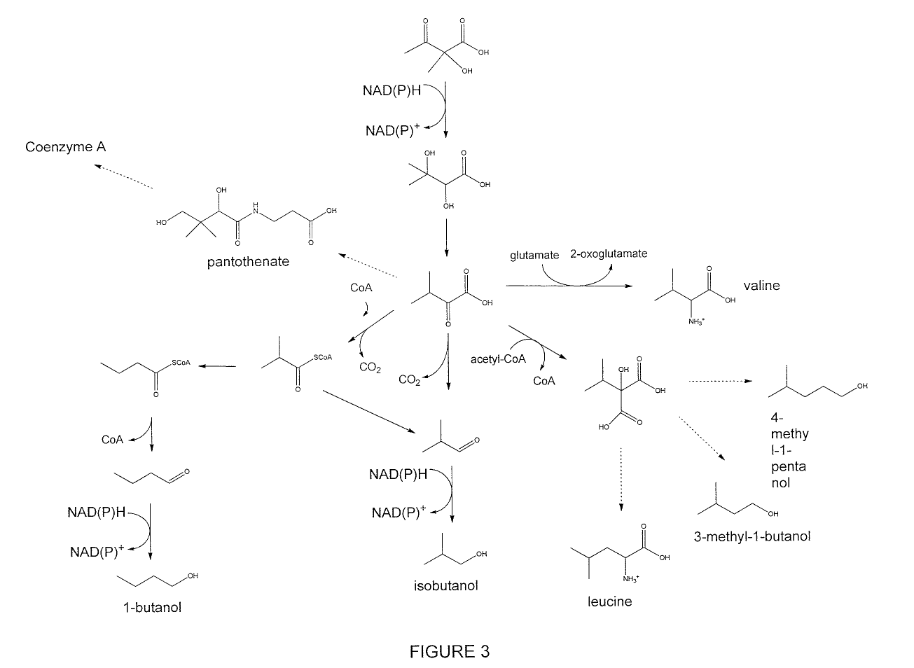

Acetolactate is formed from pyruvate by the action of the enzyme acetolactate synthase (also known as acetohydroxy acid synthase). Amongst the biosynthetic pathways using acetolactate as intermediate include pathways for the production of isobutanol, 1-butanol, valine, leucine, pantothenic acid, isobutylene, 3-methyl-1-butanol, 4-methyl-1-pentanol, and coenzyme A. Engineered biosynthetic pathways for the synthesis of acetolactate-derived metabolites are found in Table 1 and FIG. 3.

TABLE 1
Exemplary Biosynthetic Pathways Utilizing
Acetolactate as an Intermediate.

| Biosynthetic | |
|---|---|
| Pathway | Reference^{a} |
| Isobutanol | US 2009/0226991 (Feldman et al.), US 2011/0020889 |
| | (Feldman et al.), and US 2010/0143997 (Buelter et al.) |
| 1-Butanol | WO/2010/017230 (Lynch), WO/2010/031772 (Wu et al.), |
| | and KR2011002130 (Lee et al.) |
| Valine | WO/2001/021772 (Yocum et al.) and McCourt et al., 2006, |
| | Amino Acids 31: 173-210 |
| Leucine | WO/2001/021772 (Yocum et al.) and McCourt et al., 2006, |
| | Amino Acids 31: 173-210 |
| Pantothenic | WO/2001/021772 (Yocum et al.) |
| Acid | |
| 3-Methyl- | WO/2008/098227 (Liao et al.), Atsumi et al., 2008, Nature |
| 1-Butanol | 451: 86-89, and Connor et al., 2008, Appl. Environ. |
| | Microbiol. 74: 5769-5775 |
| 4-Methyl- | WO/2010/045629 (Liao et al.), Zhang et al., 2008, |
| 1-Pentanol | Proc Natl Acad Sci USA 105: 20653-20658 |
| Coenzyme A | WO/2001/021772 (Yocum et al.) |

^{a}The contents of each of the references in this table are herein incorporated by reference in their entireties for all purposes.

Each of the biosynthetic pathways listed in Table 1 shares the common 3-keto acid intermediate, acetolactate. Therefore, the product yield from these biosynthetic pathways will in part depend upon the amount of acetolactate that is available to downstream enzymes of said biosynthetic pathways.

As described herein, the present inventors have characterized the enzymatic activities responsible for the accumulation of acetoin and 2,3-butanediol, which derived from acetolactate. The present inventors have found that suppressing these newly-characterized enzymatic activities considerably reduces or eliminates the formation of 2,3-butanediol.

Reduced Accumulation of Acetoin and/or 2,3-Butanediol from Acetolactate

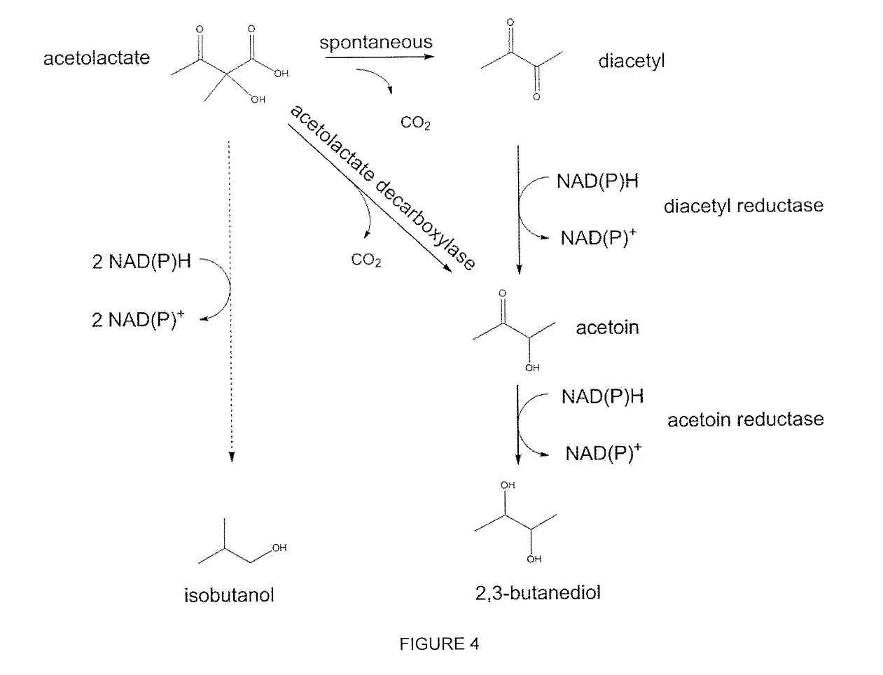

As described herein, the present inventors have found that unwanted by-products, diacetyl, acetoin, and 2,3-butanediol, can accumulate during fermentation reactions with microorganisms comprising a pathway involving an acetolactate intermediate. The conversion of acetolactate to diacetyl, acetoin, and 2,3-butanediol in the context of an isobutanol-producing metabolic pathway is illustrated in FIG. 4.

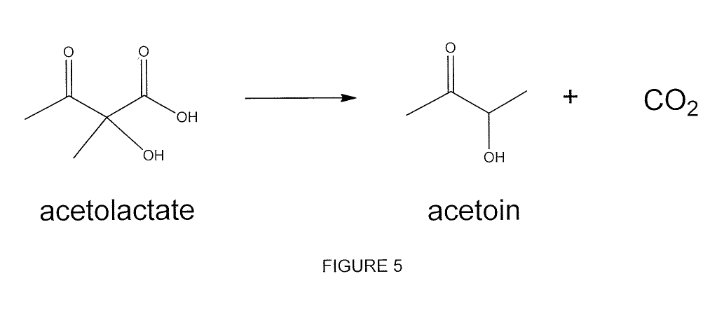

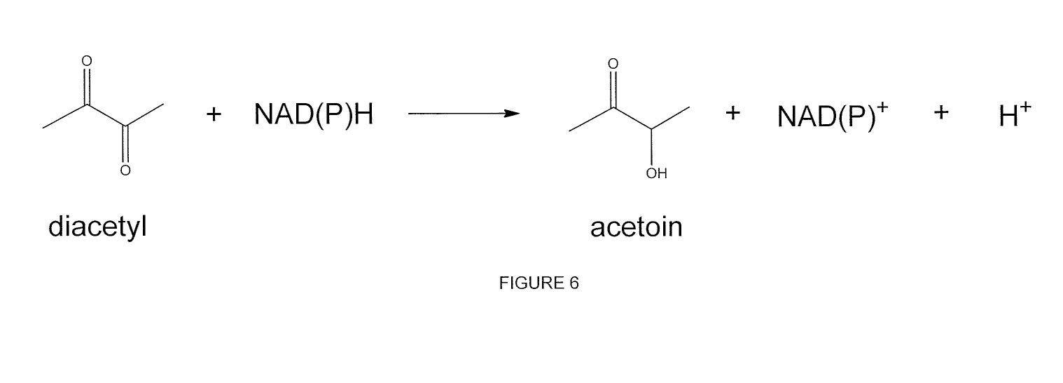

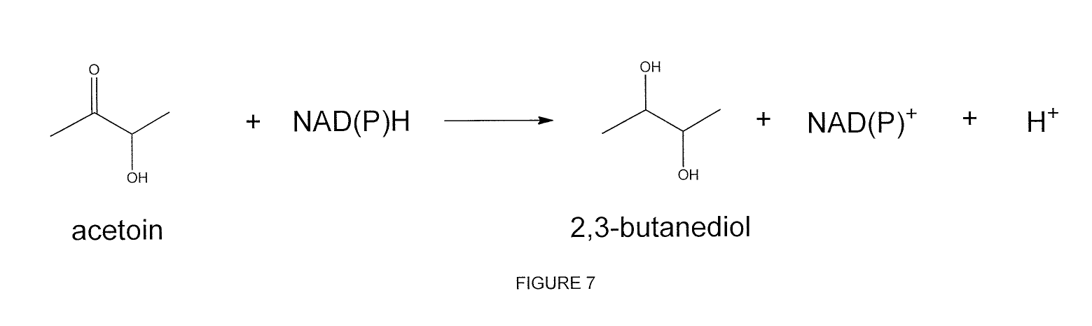

The present inventors found that the deletion of the pathway steps by which acetoin and/or 2,3-butanediol are produced helps remove competition in engineered biosynthetic pathways for reducing co-factors, which increases the NAD(P)H/NAD(P)⁺ ratio such that flux through an engineered biosynthetic pathway (e.g., an isobutanol producing metabolic pathway) may increase. As described herein, the activities of multiple enzymes are shown to be responsible for the formation of acetoin and 2,3-butanediol, including acetolactate decarboxylase (FIG. 5), diacetyl reductase (FIG. 6), and acetoin reductase (FIG. 7). Other reductase enzymes including alcohol dehydrogenases may also catalyze the reduction of diacetyl (FIG. 6) or acetoin (FIG. 7).

The present inventors describe herein multiple strategies for reducing the conversion of acetolactate to acetoin and 2,3-butanediol. As described herein, reducing the conversion of acetolactate to acetoin and/or 2,3-butanediol may help enable the increased production and/or processing of beneficial metabolites such as isobutanol, 1-butanol, valine, leucine, pantothenic acid, isobutylene, 3-methyl-1-butanol, 4-methyl-1-pentanol, and coenzyme A which are derived from biosynthetic pathways using acetolactate as an intermediate.

Accordingly, one aspect of the invention is directed to a recombinant microorganism comprising a biosynthetic pathway which uses acetolactate as an intermediate, wherein said recombinant microorganism is (a) substantially free of an enzyme catalyzing a pathway step in the conversion of acetolactate to 2,3-butanediol. In one embodiment, the enzyme catalyzes the conversion of acetolactate to acetoin. In another embodiment, the enzyme catalyzes the conversion of diacetyl to acetoin. In another embodiment, the enzyme catalyzes the conversion of acetoin to 2,3-butanediol.

In another aspect, the invention is directed to a recombinant microorganism comprising a biosynthetic pathway which uses acetolactate as an intermediate, wherein said recombinant microorganism is engineered to reduce or eliminate the expression or activity of an enzyme catalyzing the conversion of acetolactate to 2,3-butanediol. In one embodiment, the recombinant microorganism is engineered to reduce or eliminate the expression or activity of an enzyme catalyzing the conversion of acetolactate to acetoin. In another embodiment, the recombinant microorganism is engineered to reduce or eliminate the expression or activity of an enzyme catalyzing the conversion of diacetyl to acetoin. In yet another embodiment, the recombinant microorganism is engineered to reduce or eliminate the expression or activity of an enzyme catalyzing the conversion of acetoin to 2,3-butanediol.

### Reduced Conversion of Acetolactate to Acetoin

In one embodiment, the invention is directed to a recombinant microorganism comprising a biosynthetic pathway which uses acetolactate as an intermediate, wherein said recombinant microorganism is engineered to reduce or eliminate the expression or activity of one or more enzymes catalyzing the conversion of acetolactate to acetoin. In some embodiments, the enzyme catalyzing the conversion of acetolactate to acetoin is an acetolactate decarboxylase (ALDC). In an exemplary embodiment, the acetolactate decarboxylase (ALDC) is the S. cerevisiae acetolactate decarboxylase or a homolog or variant thereof.

As used herein, the term “acetolactate decarboxylase” refers to a polypeptide having an enzymatic activity that catalyzes the conversion of acetolactate to acetoin as depicted in FIG. 5. Exemplary acetolactate decarboxylases are known as EC 4.1.1.5 and are found in a variety of microorganisms. See, e.g., Godtfredsen et al., 1983, Carlsberg Res. Commun. 48: 239-247.

In one embodiment, the recombinant microorganism of the invention includes a mutation in at least one gene encoding for an acetolactate decarboxylase resulting in a reduction of acetolactate decarboxylase activity of a polypeptide encoded by said gene. In another embodiment, the recombinant microorganism includes a partial deletion of a gene encoding for an acetolactate decarboxylase gene resulting in a reduction of acetolactate decarboxylase activity of a polypeptide encoded by the gene. In another embodiment, the recombinant microorganism comprises a complete deletion of a gene encoding for an acetolactate decarboxylase resulting in a reduction of acetolactate decarboxylase activity of a polypeptide encoded by the gene. In yet another embodiment, the recombinant microorganism includes a modification of the regulatory region associated with the gene encoding for an acetolactate decarboxylase resulting in a reduction of expression of an acetolactate decarboxylase polypeptide encoded by said gene. In yet another embodiment, the recombinant microorganism comprises a modification of a transcriptional regulator resulting in a reduction of transcription of gene encoding for an acetolactate decarboxylase. In yet another embodiment, the recombinant microorganism comprises mutations in all genes encoding for an acetolactate decarboxylase resulting in a reduction of activity of a polypeptide encoded by the gene(s). In one embodiment, the acetolactate decarboxylase gene is the S. cerevisiae acetolactate decarboxylase gene or a homolog thereof. As would be understood in the art, naturally occurring homologs of acetolactate decarboxylases in yeast other than S. cerevisiae can similarly be inactivated using the methods of the present invention. Acetolactate decarboxylase homologs and methods of identifying such homologs are described herein.

As is understood by those skilled in the art, there are several additional mechanisms available for reducing or disrupting the activity of a protein such as acetolactate decarboxylase, including, but not limited to, the use of a regulated promoter, use of a weak constitutive promoter, disruption of one of the two copies of the gene in a diploid yeast, disruption of both copies of the gene in a diploid yeast, expression of an anti-sense nucleic acid, expression of an siRNA, over expression of a negative regulator of the endogenous promoter, alteration of the activity of an endogenous or heterologous gene, use of a heterologous gene with lower specific activity, the like or combinations thereof.

As described herein, the recombinant microorganisms of the present invention are engineered to produce less acetoin than an unmodified parental microorganism. In one embodiment, the recombinant microorganism produces acetoin from a carbon source at a carbon yield of less than about 20 percent. In another embodiment, the microorganism produces acetoin from a carbon source at a carbon yield of less than about 10, less than about 5, less than about 2, less than about 1, less than about 0.5, less than about 0.1, or less than about 0.01 percent.

In one embodiment, the acetoin carbon yield derived from acetolactate is reduced by at least about 50% in a recombinant microorganism as compared to a parental microorganism that does not comprise a reduction or deletion of the activity or expression of one or more acetolactate decarboxylases involved in catalyzing the conversion of acetolactate to acetoin. In another embodiment, the acetoin derived from acetolactate is reduced by at least about 60%, by at least about 65%, by at least about 70%, by at least about 75%, by at least about 80%, by at least about 85%, by at least about 90%, by at least about 95%, by at least about 99%, by at least about 99.9%, or by at least about 100% as compared to a parental microorganism that does not comprise a reduction or deletion of the activity or expression of one or more acetolactate decarboxylases involved in catalyzing the conversion of acetolactate to acetoin.

In an additional embodiment, the yield of a desirable fermentation product is increased in the recombinant microorganisms comprising a reduction or elimination of the activity or expression of one or more acetolactate decarboxylases involved in catalyzing the conversion of acetolactate to acetoin. In one embodiment, the yield of a desirable fermentation product is increased by at least about 1% as compared to a parental microorganism that does not comprise a reduction or elimination of the activity or expression of one or more acetolactate decarboxylases involved in catalyzing the conversion of acetolactate to acetoin. In another embodiment, the yield of a desirable fermentation product is increased by at least about 5%, by at least about 10%, by at least about 25%, or by at least about 50% as compared to a parental microorganism that does not comprise a reduction or elimination of the activity or expression of one or more acetolactate decarboxylases involved in catalyzing the conversion of acetolactate to acetoin. The desirable fermentation product is derived from any biosynthetic pathway in which acetolactate acts as an intermediate, including, but not limited to, isobutanol, 1-butanol, valine, leucine, pantothenic acid, isobutylene, 3-methyl-1-butanol, 4-methyl-1-pentanol, and coenzyme A.

Methods for identifying additional enzymes catalyzing the conversion of a acetolactate to acetoin are outlined as follows: endogenous yeast genes coding for potential proteins with the ability to convert acetolactate to acetoin are deleted from the genome of a yeast strain comprising a biosynthetic pathway in which acetolactate is an intermediate. These deletion strains are compared to the parent strain by fermentation and analysis of the fermentation broth for the presence and concentration of the acetoin by-product. In S. cerevisiae, deletions that reduce the production of the acetoin by-product are combined by construction of strains carrying multiple deletions. Many of these deletion strains are available commercially (for example Open Biosystems YSC1054). These deletion strains are transformed with a plasmid pGV2435 from which the ALS gene (e.g., the B. subtilis gene alsS) is expressed under the control of the CUP1 promoter. The transformants are cultivated in YPD medium containing 150 g/L glucose in shake flasks at 30° C., 75 rpm in a shaking incubator for 48 hours. After 48 h samples from the shake flasks are analyzed by HPLC for the concentration of the acetoin by-product. As would be understood in the art, naturally occurring homologs of acetolactate decarboxylases in yeast other than S. cerevisiae can similarly be inactivated using the methods of the present invention. Acetolactate decarboxylase homologs and methods of identifying such homologs are described herein.

Another way to screen the deletion library is to incubate yeast cells with acetolactate and analyze the broth for the production of the acetoin by-product.

An alternative approach to find additional endogenous activity responsible for the production of the acetoin by-product derived from acetolactate is to analyze yeast strains that overexpress the genes suspected of encoding the enzyme responsible for production of the acetoin by-product. Such strains are commercially available for many of the candidate genes listed above (for example Open Biosystems YSC3870). The ORF overexpressing strains are processed in the same way as the deletion strains. They are transformed with a plasmid for ALS expression and screened for acetoin by-product production levels. To narrow the list of possible genes causing the production of the acetoin by-product, their expression can be analyzed in fermentation samples. Genes that are not expressed during a fermentation that produced the acetoin by-product can be excluded from the list of possible targets. This analysis can be done by extraction of RNA from fermenter samples and submitting these samples to whole genome expression analysis, for example, by Roche NimbleGen.

As described herein, strains that naturally produce low levels of acetoin can also have applicability for producing increased levels of desirable fermentation products that are derived from biosynthetic pathways comprising an acetolactate intermediate. As would be understood by one skilled in the art equipped with the instant disclosure, strains that naturally produce low levels of acetoin may inherently exhibit low or undetectable levels of endogenous enzyme activity, resulting in the reduced conversion of acetolactate to acetoin, a trait favorable for the production of a desirable fermentation product such as isobutanol. Described herein are several approaches for identifying a native host microorganism which is substantially free of acetolactate decarboxylase activity. For example, one approach to finding a host microorganism which exhibits inherently low or undetectable endogenous enzyme activity responsible for the production of acetoin is to analyze yeast strains by incubating the yeast cells with acetolactate and analyze the broth for the production of acetoin.

### Reduced Conversion of Diacetyl to Acetoin

In one embodiment, the invention is directed to a recombinant microorganism comprising a biosynthetic pathway which uses acetolactate as an intermediate, wherein said recombinant microorganism is engineered to reduce or eliminate the expression or activity of one or more enzymes catalyzing the conversion of diacetyl to acetoin. Diacetyl is formed from acetolactate via the spontaneous decarboxylation of acetolactate, a process which is particularly prevalent under low pH conditions. In some embodiments, the enzyme catalyzing the conversion of diacetyl to acetoin is a diacetyl reductase. In an exemplary embodiment, the diacetyl reductase is the S. cerevisiae protein, Oye2p, or a homolog or variant thereof. In another exemplary embodiment, the diacetyl reductase is the S. cerevisiae protein, Ara1p, or a homolog or variant thereof. In yet another exemplary embodiment, the diacetyl reductase is selected from the S. cerevisiae proteins Bdh1p, Bdh2p, Erg19p, Gcy1p, Gre3p, Oye3p, Trr1p, Ypr1p, Zwf1p, and YPL088W, or homologs or variants thereof.

As used herein, the term “diacetyl reductase” refers to a polypeptide having an enzymatic activity that catalyzes the conversion of diacetyl to acetoin. Exemplary diacetyl reductases are known as EC 1.1.1.5 and are found in a variety of microorganisms, e.g., S. cerevisiae (SEQ ID NOs: 1 and 3, encoding Oye2p and Ara1p, respectively). The conversion of diacetyl to acetoin via the action of a diacetyl reductase is shown in FIG. 6.

Any method can be used to identify genes that encode for diacetyl reductases. Generally, genes that are homologous or similar to diacetyl reductases can be identified by functional, structural, and/or genetic analysis. In most cases, homologous or similar genes and/or homologous or similar enzymes will have functional, structural, or genetic similarities.

The chromosomal location of the genes encoding S. cerevisiae proteins Oye2p, Ara1p, Bdh1p, Bdh2p, Erg19p, Gcy1p, Gre3p, Oye3p, Trr1p, Ypr1p, Zwf1p, and YPL088W may be syntenic to chromosomes in many related yeast [Byrne, K. P. and K. H. Wolfe (2005) “The Yeast Gene Order Browser: combining curated homology and syntenic context reveals gene fate in polyploid species.” Genome Res. 15(10):1456-61. Scannell, D. R., K. P. Byrne, J. L. Gordon, S. Wong, and K. H. Wolfe (2006) “Multiple rounds of speciation associated with reciprocal gene loss in polyploidy yeasts.” Nature 440: 341-5. Scannell, D. R., A. C. Frank, G. C. Conant, K. P. Byrne, M. Woolfit, and K. H. Wolfe (2007)” Independent sorting-out of thousands of duplicated gene pairs in two yeast species descended from a whole-genome duplication.” Proc Natl Acad Sci USA 104: 8397-402]. Using this syntenic relationship, species-specific versions of these genes are readily identified in a variety of yeast, including but not limited to Ashbya gossypii, Candida glabrata, Kluyveromyces lactis, Kluyveromyces polysporus, Kluyveromyces thermotolerans, Kluyveromyces waltii, Saccharomyces kluyveri, Saccharomyces castelii, Saccharomyces bayanus, and Zygosaccharomyces rouxii (Table 2).

TABLE 2
Diacetyl Reductase and Acetoin Reductase Homologs.

| | |
|---|---|
| Origin | SEQ ID NO: |

Ara1p Homologs

| | |
|---|---|
| K. thermotolerans | 25 |
| K. waltii | 26 |
| S. kluyveri | 27 |
| S. castelii | 28 |
| S. bayanus | 29 |
| K. lactis | 30 |
| Z. rouxii | 31 |

Bdh1p/Bdh2p Homologs

| | |
|---|---|
| S. castelii | 32 |
| K. lactis | 33 |
| S. kluyveri | 34 |
| S. castelii | 35 |
| C. glabrata | 36 |
| K. thermotolerans | 37 |
| S. bayanus | 38 |
| S. bayanus | 39 |
| K. polysporus | 40 |
| K. lactis | 41 |
| K. waltii | 42 |

Erg19p Homologs

| | |
|---|---|
| S. castelii | 43 |
| A. gossypii | 44 |
| K. thermotolerans | 45 |
| K. lactis | 46 |
| C. glabrata | 47 |
| S. bayanus | 48 |
| S. kluyveri | 49 |
| K. waltii | 50 |
| Z. rouxii | 51 |
| K. polysporus | 52 |

Gcy1p Homologs

| | |
|---|---|
| S. kluyveri | 53 |
| Z. rouxii | 54 |
| S. castelii | 55 |
| S. bayanus | 56 |
| C. glabrata | 57 |
| K. polysporus | 58 |
| S. bayanus | 59 |
| S. castelii | 60 |
| C. glabrata | 61 |

Gre3p Homologs

| | |
|---|---|
| K. thermotolerans | 62 |
| K. waltii | 63 |
| C. glabrata | 64 |
| S. bayanus | 65 |
| A. gossypii | 66 |
| K. lactis | 67 |
| S. castelii | 68 |
| Z. rouxii | 69 |
| S. kluveri | 70 |

Oye2p Homologs

| | |
|---|---|
| S. bayanus | 71 |
| C. glabrata | 72 |
| S. castelii | 73 |

Oye3p Homologs

| | |
|---|---|
| S. bayanus | 74 |

Trr1p Homologs

| | |
|---|---|
| K. polysporus | 75 |
| S. kluyveri | 76 |
| K. waltii | 77 |
| C. glabrata | 78 |
| S. bayanus | 79 |
| A. gossypii | 80 |
| C. glabrata | 81 |
| S. castelii | 82 |
| K. polysporus | 83 |
| S. bayanus | 84 |
| Z. rouxii | 85 |
| K. thermotolerans | 86 |
| K. lactis | 87 |
| S. castelii | 88 |

Ypr1p Homologs

| | |
|---|---|
| S. kluyveri | 89 |
| Z. rouxii | 90 |
| A. gossypii | 91 |
| C. glabrata | 92 |
| S. bayanus | 93 |
| S. castelii | 94 |
| K. thermotolerans | 95 |
| K. polysporus | 96 |
| K. waltii | 97 |
| S. bayanus | 98 |
| S. castelii | 99 |
| C. glabrata | 100 |

Zwf1p Homologs

| | |
|---|---|
| K. lactis | 101 |
| S. castelii | 102 |
| S. kluyveri | 103 |
| A. gossypii | 104 |
| K. polysporus | 105 |
| C. glabrata | 106 |
| K. thermotolerans | 107 |
| K. waltii | 108 |
| Z. rouxii | 109 |

YPL088W Homologs

| | |
|---|---|
| S. bayanus | 110 |
| S. castelii | 111 |
| S. kluyveri | 112 |

In one embodiment, the recombinant microorganism of the invention includes a mutation in at least one gene encoding for a diacetyl reductase resulting in a reduction of diacetyl reductase activity of a polypeptide encoded by said gene.

In another embodiment, the recombinant microorganism includes a partial deletion of a gene encoding for a diacetyl reductase gene resulting in a reduction of diacetyl reductase activity of a polypeptide encoded by the gene. In another embodiment, the recombinant microorganism comprises a complete deletion of a gene encoding for a diacetyl reductase resulting in a reduction of diacetyl reductase activity of a polypeptide encoded by the gene. In yet another embodiment, the recombinant microorganism includes a modification of the regulatory region associated with the gene encoding for a diacetyl reductase resulting in a reduction of expression of a diacetyl reductase polypeptide encoded by said gene. In yet another embodiment, the recombinant microorganism comprises a modification of a transcriptional regulator resulting in a reduction of transcription of gene encoding for a diacetyl reductase. In yet another embodiment, the recombinant microorganism comprises mutations in all genes encoding for a diacetyl reductase resulting in a reduction of activity of a polypeptide encoded by the gene(s). In an exemplary embodiment, the diacetyl reductase is the S. cerevisiae protein, Oye2p, or a homolog or variant thereof. In another exemplary embodiment, the diacetyl reductase is the S. cerevisiae protein, Ara1p, or a homolog or variant thereof. In yet another exemplary embodiment, the diacetyl reductase is selected from the S. cerevisiae proteins Bdh1p, Bdh2p, Erg19p, Gcy1p, Gre3p, Oye3p, Trr1p, Ypr1p, Zwf1p, and YPL088W, or homologs or variants thereof. As would be understood in the art, naturally occurring homologs of diacetyl reductases such as Oye2p, Ara1p, Bdh1p, Bdh2p, Erg19p, Gcy1p, Gre3p, Oye3p, Trr1p, Ypr1p, Zwf1p, and YPL088W in yeast other than S. cerevisiae can similarly be inactivated using the methods of the present invention. Diacetyl reductase homologs and methods of identifying such diacetyl reductase homologs are described herein.

As is understood by those skilled in the art, there are several additional mechanisms available for reducing or disrupting the activity of a protein such as diacetyl reductase, including, but not limited to, the use of a regulated promoter, use of a weak constitutive promoter, disruption of one of the two copies of the gene in a diploid yeast, disruption of both copies of the gene in a diploid yeast, expression of an anti-sense nucleic acid, expression of an siRNA, over expression of a negative regulator of the endogenous promoter, alteration of the activity of an endogenous or heterologous gene, use of a heterologous gene with lower specific activity, the like or combinations thereof.

As described herein, the recombinant microorganisms of the present invention are engineered to produce less acetoin than an unmodified parental microorganism. In one embodiment, the recombinant microorganism produces acetoin from a carbon source at a carbon yield of less than about 20 percent. In another embodiment, the microorganism produces acetoin from a carbon source at a carbon yield of less than about 10, less than about 5, less than about 2, less than about 1, less than about 0.5, less than about 0.1, or less than about 0.01 percent.

In one embodiment, the acetoin carbon yield derived from diacetyl is reduced by at least about 50% in a recombinant microorganism as compared to a parental microorganism that does not comprise a reduction or deletion of the activity or expression of one or more diacetyl reductases involved in catalyzing the conversion of diacetyl to acetoin. In another embodiment, the acetoin derived from diacetyl is reduced by at least about 60%, by at least about 65%, by at least about 70%, by at least about 75%, by at least about 80%, by at least about 85%, by at least about 90%, by at least about 95%, by at least about 99%, by at least about 99.9%, or by at least about 100% as compared to a parental microorganism that does not comprise a reduction or deletion of the activity or expression of one or more diacetyl reductases involved in catalyzing the conversion of diacetyl to acetoin.

In an additional embodiment, the yield of a desirable fermentation product is increased in the recombinant microorganisms comprising a reduction or elimination of the activity or expression of one or more diacetyl reductases involved in catalyzing the conversion of diacetyl to acetoin. In one embodiment, the yield of a desirable fermentation product is increased by at least about 1% as compared to a parental microorganism that does not comprise a reduction or elimination of the activity or expression of one or more diacetyl reductases involved in catalyzing the conversion of diacetyl to acetoin. In another embodiment, the yield of a desirable fermentation product is increased by at least about 5%, by at least about 10%, by at least about 25%, or by at least about 50% as compared to a parental microorganism that does not comprise a reduction or elimination of the activity or expression of one or more diacetyl reductases involved in catalyzing the conversion of diacetyl to acetoin. The desirable fermentation product is derived from any biosynthetic pathway in which acetolactate acts as an intermediate, including, but not limited to, isobutanol, 1-butanol, valine, leucine, pantothenic acid, isobutylene, 3-methyl-1-butanol, 4-methyl-1-pentanol, and coenzyme A.

Methods for identifying additional enzymes catalyzing the conversion of a diacetyl to acetoin are outlined as follows: endogenous yeast genes coding for diacetyl reductases, which could include any enzyme catalyzing the reaction shown in FIG. 6 are deleted from the genome of a yeast strain comprising a biosynthetic pathway in which acetolactate is an intermediate. These deletion strains are compared to the parent strain by fermentation and analysis of the fermentation broth for the presence and concentration of the acetoin by-product. In S. cerevisiae, deletions that reduce the production of the acetoin by-product are combined by construction of strains carrying multiple deletions. Candidate genes can include, but are not limited to, ARA1 (NADP+ dependent arabinose dehydrogenase), BDH1 (NAD-dependent (R,R)-butanediol dehydrogenase), BDH2 (Putative medium-chain alcohol dehydrogenase), ERG19 (Mevalonate pyrophosphate decarboxylase), GCY/(Putative NADP(+) coupled glycerol dehydrogenase), GRE3 (Aldose reductase), OYEZ (NADPH oxidoreductase), OYE3 (NADPH oxidoreductase), TRR1 (thioredoxin reductase), YPL088W (Putative aryl alcohol dehydrogenase), YPR1 (NADPH-dependent aldo-keto reductase), and ZWF1 (Glucose-6-phosphate dehydrogenase). These candidate genes are deleted from strains expressing ALS, for example by integrating an ALS gene (e.g., the B. subtilis alsS) expressed under a constitutive promoter. The transformants are cultivated in an appropriate culture medium under appropriate conditions. For example, the transformants are cultivated in YPD medium containing 80 g/L glucose in shake flasks at 30° C., 250 rpm in a shaking incubator for 24 hours, then at 30° C., 75 rpm. After 72 h, samples from the shake flasks are analyzed by HPLC for the concentration of the acetoin by-product. As would be understood in the art, naturally occurring homologs of diacetyl reductases such as Oye2p, Ara1p, Bdh1p, Bdh2p, Erg19p, Gcy1p, Gre3p, Oye3p, Trr1p, Ypr1p, Zwf1p, and YPL088W in yeast other than S. cerevisiae can similarly be inactivated using the methods of the present invention. Diacetyl reductase homologs and methods of identifying such diacetyl reductase homologs are described herein. Many of these deletion strains are also available commercially (for example Open Biosystems YSC1054). These deletion strains are transformed with a plasmid pGV2435 from which the ALS gene (e.g., the B. subtilis alsS) is expressed under the control of the CUP1 promoter. The transformants are cultivated in YPD medium containing 150 g/L glucose in shake flasks at 30° C., 75 rpm in a shaking incubator for 48 hours. After 48 h samples from the shake flasks are analyzed by HPLC for the concentration of the acetoin by-product.

Another way to screen the deletion library is to incubate yeast cells with diacetyl and analyze the broth for the production of the acetoin by-product.

Some of the listed genes are the result of tandem duplication or whole genome duplication events and are expected to have similar substrate specificities. Examples are YAL061W (BDH1), and YAL060W (BDH2), YDR368W (YPR1) and YOR120W (GCY1). Deletion of just one of the duplicated genes is likely not to result in a phenotype. These gene pairs have to be analyzed in strains carrying deletions in both genes.

An alternative approach to find additional endogenous activity responsible for the production of the acetoin by-product derived from diacetyl is to analyze yeast strains that overexpress the genes suspected of encoding the enzyme responsible for production of the acetoin by-product. Such strains are commercially available for many of the candidate genes listed above (for example Open Biosystems YSC3870). The ORF overexpressing strains are transformed with a plasmid for ALS expression and screened for acetoin by-product production levels. To narrow the list of possible genes causing the production of the acetoin by-product, their expression can be analyzed in fermentation samples. Genes that are not expressed during a fermentation that produced the acetoin by-product can be excluded from the list of possible targets. This analysis can be done by extraction of RNA from fermenter samples and submitting these samples to whole genome expression analysis, for example, by Roche NimbleGen.

As described herein, strains that naturally produce low levels of acetoin can also have applicability for producing increased levels of desirable fermentation products that are derived from biosynthetic pathways comprising an acetolactate intermediate. As would be understood by one skilled in the art equipped with the instant disclosure, strains that naturally produce low levels of acetoin may inherently exhibit low or undetectable levels of endogenous enzyme activity, resulting in the reduced conversion of diacetyl to acetoin, a trait favorable for the production of a desirable fermentation product such as isobutanol. Described herein are several approaches for identifying a native host microorganism which is substantially free of diacetyl reductase activity. For example, one approach to finding a host microorganism which exhibits inherently low or undetectable endogenous enzyme activity responsible for the production of acetoin is to analyze yeast strains by incubating the yeast cells with diacetyl and analyze the broth for the production of acetoin.

### Reduced Conversion of Acetoin to 2,3-Butanediol

In one embodiment, the invention is directed to a recombinant microorganism comprising a biosynthetic pathway which uses acetolactate as an intermediate, wherein said recombinant microorganism is engineered to reduce or eliminate the expression or activity of one or more enzymes catalyzing the conversion of acetoin to 2,3-butanediol. In some embodiments, the enzyme catalyzing the conversion of acetoin to 2,3-butanediol is an acetoin reductase. In an exemplary embodiment, the acetoin reductase is the S. cerevisiae protein, Bdh1p, or a homolog or variant thereof. In another exemplary embodiment, acetoin reductase is the S. cerevisiae protein, Bdh2p, or a homolog or variant thereof. In yet another exemplary embodiment, the acetoin reductase is the S. cerevisiae protein, Ara1p, or a homolog or variant thereof. In yet another exemplary embodiment, the acetoin reductase is selected from the S. cerevisiae proteins Erg19p, Gcy1p, Gre3p, Oye2p, Oye3p, Trr1p, Ypr1p, Zwf1p, and YPL088W, or homologs or variants thereof.

The terms “acetoin reductase” and “2,3-butanediol dehydrogenase” are used interchangeably herein to refer to a polypeptide having an enzymatic activity that catalyzes the conversion of acetoin to 2,3-butanediol. Exemplary acetoin are known as EC 1.1.1.4 and are found in a variety of microorganisms, e.g., S. cerevisiae (SEQ ID NOs: 5 and 7, encoding Bdh1p and Bdh2p, respectively). The conversion of acetoin to 2,3-butanediol via the action of an acetoin reductase is shown in FIG. 7.

Any method can be used to identify genes that encode for acetoin reductases. Generally, genes that are homologous or similar to acetoin reductases can be identified by functional, structural, and/or genetic analysis. In most cases, homologous or similar genes and/or homologous or similar enzymes will have functional, structural, or genetic similarities.

The chromosomal location of the genes encoding S. cerevisiae proteins Bdh1p, Bdh2p, Ara1p, Erg19p, Gcy1p, Gre3p, Oye2p, Oye3p, Trr1p, Ypr1p, Zwf1p, and YPL088W may be syntenic to chromosomes in many related yeast [Byrne, K. P. and K. H. Wolfe (2005) “The Yeast Gene Order Browser: combining curated homology and syntenic context reveals gene fate in polyploid species.” Genome Res. 15(10):1456-61. Scannell, D. R., K. P. Byrne, J. L. Gordon, S. Wong, and K. H. Wolfe (2006) “Multiple rounds of speciation associated with reciprocal gene loss in polyploidy yeasts.” Nature 440: 341-5. Scannell, D. R., A. C. Frank, G. C. Conant, K. P. Byrne, M. Woolfit, and K. H. Wolfe (2007)” Independent sorting-out of thousands of duplicated gene pairs in two yeast species descended from a whole-genome duplication.” Proc Natl Acad Sci USA 104: 8397-402]. Using this syntenic relationship, species-specific versions of these genes are readily identified in a variety of yeast, including but not limited to Ashbya gossypii, Candida glabrata, Kluyveromyces lactis, Kluyveromyces polysporus, Kluyveromyces thermotolerans, Kluyveromyces waltii, Saccharomyces kluyveri, Saccharomyces castelii, Saccharomyces bayanus, and Zygosaccharomyces rouxii (Table 2).

In one embodiment, the recombinant microorganism of the invention includes a mutation in at least one gene encoding for an acetoin reductase resulting in a reduction of acetoin reductase activity of a polypeptide encoded by said gene. In another embodiment, the recombinant microorganism includes a partial deletion of a gene encoding for an acetoin reductase gene resulting in a reduction of acetoin reductase activity of a polypeptide encoded by the gene. In another embodiment, the recombinant microorganism comprises a complete deletion of a gene encoding for an acetoin reductase resulting in a reduction of acetoin reductase activity of a polypeptide encoded by the gene. In yet another embodiment, the recombinant microorganism includes a modification of the regulatory region associated with the gene encoding for an acetoin reductase resulting in a reduction of expression of an acetoin reductase polypeptide encoded by said gene. In yet another embodiment, the recombinant microorganism comprises a modification of a transcriptional regulator resulting in a reduction of transcription of gene encoding for an acetoin reductase. In yet another embodiment, the recombinant microorganism comprises mutations in all genes encoding for an acetoin reductase resulting in a reduction of activity of a polypeptide encoded by the gene(s). In an exemplary embodiment, the acetoin reductase is the S. cerevisiae protein, Bdh1p, or a homolog or variant thereof. In another exemplary embodiment, acetoin reductase is the S. cerevisiae protein, Bdh2p, or a homolog or variant thereof. In yet another exemplary embodiment, the acetoin reductase is the S. cerevisiae protein, Ara1p, or a homolog or variant thereof. In yet another exemplary embodiment, the acetoin reductase is selected from the S. cerevisiae proteins Erg19p, Gcy1p, Gre3p, Oye2p, Oye3p, Trr1p, Ypr1p, Zwf1p, and YPL088W, or homologs or variants thereof. As would be understood in the art, naturally occurring homologs of acetoin reductases such as Bdh1p, Bdh2p, Ara1p, Erg19p, Gcy1p, Gre3p, Oye2p, Oye3p, Trr1p, Ypr1p, Zwf1p, and/or YPL088W in yeast other than S. cerevisiae can similarly be inactivated using the methods of the present invention. Acetoin reductase homologs and methods of identifying such acetoin reductase homologs are described herein.

As is understood by those skilled in the art, there are several additional mechanisms available for reducing or disrupting the activity of a protein such as acetoin reductase, including, but not limited to, the use of a regulated promoter, use of a weak constitutive promoter, disruption of one of the two copies of the gene in a diploid yeast, disruption of both copies of the gene in a diploid yeast, expression of an anti-sense nucleic acid, expression of an siRNA, over expression of a negative regulator of the endogenous promoter, alteration of the activity of an endogenous or heterologous gene, use of a heterologous gene with lower specific activity, the like or combinations thereof.

As described herein, the recombinant microorganisms of the present invention are engineered to produce less 2,3-butanediol than an unmodified parental microorganism. In one embodiment, the recombinant microorganism produces 2,3-butanediol from a carbon source at a carbon yield of less than about 20 percent. In another embodiment, the microorganism produces 2,3-butanediol from a carbon source at a carbon yield of less than about 10, less than about 5, less than about 2, less than about 1, less than about 0.5, less than about 0.1, or less than about 0.01 percent.

In one embodiment, the 2,3-butanediol carbon yield derived from acetoin is reduced by at least about 50% in a recombinant microorganism as compared to a parental microorganism that does not comprise a reduction or deletion of the activity or expression of one or more acetoin reductases involved in catalyzing the conversion of acetoin to 2,3-butanediol. In another embodiment, the 2,3-butanediol derived from acetoin is reduced by at least about 60%, by at least about 65%, by at least about 70%, by at least about 75%, by at least about 80%, by at least about 85%, by at least about 90%, by at least about 95%, by at least about 99%, by at least about 99.9%, or by at least about 100% as compared to a parental microorganism that does not comprise a reduction or deletion of the activity or expression of one or more acetoin reductases involved in catalyzing the conversion of acetoin to 2,3-butanediol.

In an additional embodiment, the yield of a desirable fermentation product is increased in the recombinant microorganisms comprising a reduction or elimination of the activity or expression of one or more acetoin reductases involved in catalyzing the conversion of acetoin to 2,3-butanediol. In one embodiment, the yield of a desirable fermentation product is increased by at least about 1% as compared to a parental microorganism that does not comprise a reduction or elimination of the activity or expression of one or more acetoin reductases involved in catalyzing the conversion of acetoin to 2,3-butanediol. In another embodiment, the yield of a desirable fermentation product is increased by at least about 5%, by at least about 10%, by at least about 25%, or by at least about 50% as compared to a parental microorganism that does not comprise a reduction or elimination of the activity or expression of one or more acetoin reductases involved in catalyzing the conversion of acetoin to 2,3-butanediol. The desirable fermentation product is derived from any biosynthetic pathway in which acetolactate acts as an intermediate, including, but not limited to, isobutanol, 1-butanol, valine, leucine, pantothenic acid, isobutylene, 3-methyl-1-butanol, 4-methyl-1-pentanol, and coenzyme A.

Methods for identifying additional enzymes catalyzing the conversion of acetoin to 2,3-butanediol are outlined as follows: endogenous yeast genes coding for acetoin reductases, which could include any enzyme catalyzing the reaction shown in FIG. 7 are deleted from the genome of a yeast strain comprising a biosynthetic pathway in which acetolactate is an intermediate. These deletion strains are compared to the parent strain by fermentation and analysis of the fermentation broth for the presence and concentration of the 2,3-butanediol by-product. In S. cerevisiae, deletions that reduce the production of the 2,3-butanediol by-product are combined by construction of strains carrying multiple deletions. Candidate genes can include, but are not limited to, ARA1 (NADP+ dependent arabinose dehydrogenase), BDH1 (NAD-dependent (R,R)-butanediol dehydrogenase), BDH2 (Putative medium-chain alcohol dehydrogenase), ERG19 (Mevalonate pyrophosphate decarboxylase), GCY1 (Putative NADP(+) coupled glycerol dehydrogenase), GRE3 (Aldose reductase), OYE2 (NADPH oxidoreductase), OYES (NADPH oxidoreductase), TRR1 (thioredoxin reductase), YPL088W (Putative aryl alcohol dehydrogenase), YPR1 (NADPH-dependent aldo-keto reductase), and ZWF1 (Glucose-6-phosphate dehydrogenase). These candidate genes are deleted from strains expressing ALS, for example by integrating an ALS gene (e.g., the B. subtilis alsS) expressed under a constitutive promoter. The transformants are cultivated in an appropriate culture medium under appropriate conditions. For example, the transformants are cultivated in YPD medium containing 80 g/L glucose in shake flasks at 30° C., 250 rpm in a shaking incubator for 24 hours, then at 30° C., 75 rpm. After 72 h, samples from the shake flasks are analyzed by HPLC for the concentration of the 2,3-butanediol by-product. As would be understood in the art, naturally occurring homologs of acetoin reductases such as Bdh1p, Bdh2p, Ara1p, Erg19p, Gcy1p, Gre3p, Oye2p, Oye3p, Trr1p, Ypr1p, Zwf1p, and/or YPL088W in yeast other than S. cerevisiae can similarly be inactivated using the methods of the present invention. Acetoin reductase homologs and methods of identifying such acetoin reductase homologs are described herein. Many of these deletion strains are also available commercially (for example Open Biosystems YSC1054). These deletion strains are transformed with a plasmid pGV2435 from which the ALS gene (e.g., the B. subtilis alsS) is expressed under the control of the CUP1 promoter. The transformants are cultivated in YPD medium containing 150 g/L glucose in shake flasks at 30° C., 75 rpm in a shaking incubator for 48 hours. After 48 h samples from the shake flasks are analyzed by HPLC for the concentration of the 2,3-butanediol by-product.

Another way to screen the deletion library is to incubate yeast cells with acetoin and analyze the broth for the production of the 2,3-butanediol by-product.

Some of the listed genes are the result of tandem duplication or whole genome duplication events and are expected to have similar substrate specificities. Examples are YAL061W (BDH1), and YAL060W (BDH2), YDR368W (YPR1) and YOR120W (GCY1). Deletion of just one of the duplicated genes is likely not to result in a phenotype. These gene pairs have to be analyzed in strains carrying deletions in both genes.

An alternative approach to find additional endogenous activity responsible for the production of the 2,3-butanediol by-product derived from acetoin is to analyze yeast strains that overexpress the genes suspected of encoding the enzyme responsible for production of the 2,3-butanediol by-product. Such strains are commercially available for many of the candidate genes listed above (for example Open Biosystems YSC3870). The ORF overexpressing strains are transformed with a plasmid for ALS expression and screened for 2,3-butanediol by-product production levels. To narrow the list of possible genes causing the production of the 2,3-butanediol by-product, their expression can be analyzed in fermentation samples. Genes that are not expressed during a fermentation that produced the 2,3-butanediol by-product can be excluded from the list of possible targets. This analysis can be done by extraction of RNA from fermenter samples and submitting these samples to whole genome expression analysis, for example, by Roche NimbleGen. As described herein, strains that naturally produce low levels of 2,3-butanediol can also have applicability for producing increased levels of desirable fermentation products that are derived from biosynthetic pathways comprising an acetolactate intermediate. As would be understood by one skilled in the art equipped with the instant disclosure, strains that naturally produce low levels of 2,3-butanediol may inherently exhibit low or undetectable levels of endogenous enzyme activity, resulting in the reduced conversion of acetoin to 2,3-butanediol, a trait favorable for the production of a desirable fermentation product such as isobutanol. Described herein are several approaches for identifying a native host microorganism which is substantially free of acetoin reductase activity. For example, one approach to finding a host microorganism which exhibits inherently low or undetectable endogenous enzyme activity responsible for the production of 2,3-butanediol is to analyze yeast strains by incubating the yeast cells with acetoin and analyze the broth for the production of 2,3-butanediol.

Reduced Activity of Multiple Enzymes Involved in Production of 2,3-Butanediol from Acetolactate

As would be understood by one skilled in the art equipped with the instant disclosure, the expression or activity of multiple enzymes involved in catalyzing the conversion of acetolactate to 2,3-butanediol can be reduced or eliminated. In one embodiment, the expression or activity of at least one enzyme catalyzing the conversion of acetolactate to acetoin is reduced or eliminated. In another embodiment, the expression or activity of at least one enzyme catalyzing the conversion of diacetyl to acetoin is reduced or eliminated. In yet another embodiment, the expression or activity of at least one enzyme catalyzing the conversion of diacetyl to acetoin is reduced or eliminated.

In one exemplary embodiment, the invention is directed to a recombinant microorganism comprising a biosynthetic pathway which uses acetolactate as an intermediate, wherein the recombinant microorganism is engineered to reduce or eliminate the expression or activity of at least two of the following: (i) one or more enzymes catalyzing the conversion of acetolactate to acetoin; (ii) one or more enzymes catalyzing the conversion of diacetyl to acetoin; and/or (iii) one or more enzymes catalyzing the conversion of acetoin to 2,3-butanediol. In one embodiment, the enzyme catalyzing the conversion of acetolactate to acetoin is an acetolactate decarboxylase (ALDC). In an exemplary embodiment, the acetolactate decarboxylase is the S. cerevisiae acetolactate decarboxylase or a homolog or variant thereof. In one embodiment, the enzyme catalyzing the conversion of diacetyl to acetoin is a diacetyl reductase. In an exemplary embodiment, the diacetyl reductase is the S. cerevisiae protein, Oye2p, or a homolog or variant thereof. In another exemplary embodiment, the diacetyl reductase is the S. cerevisiae protein, Ara1p, or a homolog or variant thereof. In yet another exemplary embodiment, the diacetyl reductase is selected from the S. cerevisiae proteins Bdh1p, Bdh2p, Erg19p, Gcy1p, Gre3p, Oye3p, Trr1p, Ypr1p, Zwf1p, and YPL088W, or homologs or variants thereof. In one embodiment, the enzyme catalyzing the conversion of acetoin to 2,3-butanediol is an acetoin reductase. In an exemplary embodiment, the acetoin reductase is the S. cerevisiae protein, Bdh1p, or a homolog or variant thereof. In another exemplary embodiment, acetoin reductase is the S. cerevisiae protein, Bdh2p, or a homolog or variant thereof. In yet another exemplary embodiment, the acetoin reductase is the S. cerevisiae protein, Ara1p, or a homolog or variant thereof. In yet another exemplary embodiment, the acetoin reductase is selected from the S. cerevisiae proteins Erg19p, Gcy1p, Gre3p, Oye2p, Oye3p, Trr1p, Ypr1p, Zwf1p, and YPL088W, or homologs or variants thereof. In some embodiments, the expression or activity of one or more acetolactate decarboxylases, one or more diacetyl reductases, and one or more acetoin reductases is reduced or eliminated.

In another exemplary embodiment, the invention is directed to a recombinant microorganism comprising a biosynthetic pathway which uses acetolactate as an intermediate, wherein the recombinant microorganism is engineered to reduce or eliminate the expression or activity of all of the following: (i) one or more enzymes catalyzing the conversion of acetolactate to acetoin; (ii) one or more enzymes catalyzing the conversion of diacetyl to acetoin; and (iii) one or more enzymes catalyzing the conversion of acetoin to 2,3-butanediol. In one embodiment, the enzyme catalyzing the conversion of acetolactate to acetoin is an acetolactate decarboxylase (ALDC). In an exemplary embodiment, the acetolactate decarboxylase is the S. cerevisiae acetolactate decarboxylase or a homolog or variant thereof. In one embodiment, the enzyme catalyzing the conversion of diacetyl to acetoin is a diacetyl reductase. In an exemplary embodiment, the diacetyl reductase is the S. cerevisiae protein, Oye2p, or a homolog or variant thereof. In another exemplary embodiment, the diacetyl reductase is the S. cerevisiae protein, Ara1p, or a homolog or variant thereof. In yet another exemplary embodiment, the diacetyl reductase is selected from the S. cerevisiae proteins Bdh1p, Bdh2p, Erg19p, Gcy1p, Gre3p, Oye3p, Trr1p, Ypr1p, Zwf1p, and YPL088W, or homologs or variants thereof. In one embodiment, the enzyme catalyzing the conversion of acetoin to 2,3-butanediol is an acetoin reductase. In an exemplary embodiment, the acetoin reductase is the S. cerevisiae protein, Bdh1p, or a homolog or variant thereof. In another exemplary embodiment, acetoin reductase is the S. cerevisiae protein, Bdh2p, or a homolog or variant thereof. In yet another exemplary embodiment, the acetoin reductase is the S. cerevisiae protein, Ara1p, or a homolog or variant thereof. In yet another exemplary embodiment, the acetoin reductase is selected from the S. cerevisiae proteins Erg19p, Gcy1p, Gre3p, Oye2p, Oye3p, Trr1p, Ypr1p, Zwf1p, and YPL088W, or homologs or variants thereof.

### Further Reductions in Enzymatic Activity

The recombinant microorganisms described herein which produce a beneficial metabolite derived from a biosynthetic pathway using acetolactate as an intermediate may be further engineered to reduce or eliminate enzymatic activity for the conversion of pyruvate to products other acetolactate. In one embodiment, the enzymatic activity of pyruvate decarboxylase (PDC), lactate dehydrogenase (LDH), pyruvate oxidase, pyruvate dehydrogenase, and/or glycerol-3-phosphate dehydrogenase (GPD) is reduced or eliminated.

In a specific embodiment, the beneficial metabolite is produced in a recombinant PDC-minus GPD-minus yeast microorganism that overexpresses an acetolactate synthase (ALS) gene. In another specific embodiment, the ALS is encoded by the B. subtilis alsS.

The recombinant microorganisms described herein that produce a beneficial metabolite derived from a biosynthetic pathway using acetolactate as an intermediate may be further engineered to reduce or eliminate enzymatic activity for the conversion of acetolactate to additional unwanted by-products. One such by-product is DH2 MB, described in commonly owned and co-pending publication, US 2011/0201090, which is herein incorporated by reference in its entirety for all purposes. The production of this by-product can be reduced by engineering the recombinant microorganism to reduce or eliminate the expression or activity of a 3-keto acid reductase (3-KAR). In one embodiment, the 3-ketoacid reductase is the S. cerevisiae YMR226C (SEQ ID NO: 113) protein or a homolog or variant thereof. In one embodiment, the homolog may be selected from the group consisting of Vanderwaltomzyma polyspora (SEQ ID NO: 114), Saccharomyces castelii (SEQ ID NO: 115), Candida glabrata (SEQ ID NO: 116), Saccharomyces bayanus (SEQ ID NO: 117), Zygosaccharomyces rouxii (SEQ ID NO: 118), K. lactis (SEQ ID NO: 119), Ashbya gossypii (SEQ ID NO: 120), Saccharomyces kluyveri (SEQ ID NO: 121), Kluyveromyces thermotolerans (SEQ ID NO: 122), Kluyveromyces waltii (SEQ ID NO: 123), Pichia stipitis (SEQ ID NO: 124), Debaromyces hansenii (SEQ ID NO: 125), Pichia pastoris (SEQ ID NO: 126), Candida dubliniensis (SEQ ID NO: 127), Candida albicans (SEQ ID NO: 128), Yarrowia lipolytica (SEQ ID NO: 129), Issatchenkia orientalis (SEQ ID NO: 130), Aspergillus nidulans (SEQ ID NO: 131), Aspergillus niger (SEQ ID NO: 132), Neurospora crassa (SEQ ID NO: 133), Schizosaccharomyces pombe (SEQ ID NO: 134), and Kluyveromyces marxianus (SEQ ID NO: 135).

As shown in FIG. 3, some biosynthetic pathways which comprise acetolactate as an intermediate may also utilize an aldehyde as an intermediate. In one embodiment, the expression or activity of an enzyme converting the aldehyde intermediate to an unwanted acid by-product may be reduced or eliminated. In some embodiments, the enzyme converting the aldehyde intermediate to an unwanted acid by-product is an aldehyde dehydrogenase (ALDH). In one embodiment, the aldehyde dehydrogenase is encoded by a gene selected from the group consisting of ALD2, ALD3, ALD4, ALD5, ALD6, and HFD1, and homologs and variants thereof. In an exemplary embodiment, the aldehyde dehydrogenase is the S. cerevisiae ALD6 (SEQ ID NO: 136) protein. In some embodiments, the aldehyde dehydrogenase is the S. cerevisiae ALD6 (SEQ ID NO: 136) protein or a homolog or variant thereof. In one embodiment, the homolog is selected from the group consisting of Saccharomyces castelli (SEQ ID NO: 137), Candida glabrata (SEQ ID NO: 138), Saccharomyces bayanus (SEQ ID NO: 139), Kluyveromyces lactis (SEQ ID NO: 140), Kluyveromyces thermotolerans (SEQ ID NO: 141), Kluyveromyces waltii (SEQ ID NO: 142), Saccharomyces cerevisiae YJ789 (SEQ ID NO: 143), Saccharomyces cerevisiae JAY291 (SEQ ID NO: 144), Saccharomyces cerevisiae EC1118 (SEQ ID NO: 145), Saccharomyces cerevisiae DBY939 (SEQ ID NO: 146), Saccharomyces cerevisiae AWR11631 (SEQ ID NO: 147), Saccharomyces cerevisiae RM11-1a (SEQ ID NO: 148), Pichia pastoris (SEQ ID NO: 149), Kluyveromyces marxianus (SEQ ID NO: 150), Schizosaccharomyces pombe (SEQ ID NO: 151), and Schizosaccharomyces pombe (SEQ ID NO: 152). Methods for reducing or eliminating the expression or activity of an aldehyde dehydrogenase are further described in commonly owned and co-pending U.S. Application Serial No. 2011/0201090, which is herein incorporated by reference in its entirety for all purposes.

Reduced Transporter Expression and/or Activity

The recombinant microorganisms described herein that produce a beneficial metabolite derived from a biosynthetic pathway using acetolactate as an intermediate may be further engineered to reduce the activity and/or expression of one or more endogenous transporter proteins, including but not limited to, endogenous transporter proteins involved in the secretion of acetolactate. Exemplary endogenous transporter proteins are described in commonly owned and co-pending publication, WO/2011/153144, which is herein incorporated by reference in its entirety for all purposes. In one embodiment, the recombinant microorganism comprising a biosynthetic pathway which uses acetolactate as an intermediate may be engineered to reduce the activity and/or expression of one or more endogenous transporter proteins selected from the group consisting of Opt1p, Opt2p, YGL141W, Adp1p, Arb1p, Atm1p, Aus1p, Bpt1p, Mdl1p, Mdl2p, Nft1p, Pdr5p, Pdr10p, Pdr11p, Pdr12p, Pdr15p, Pdr18p, Pxa1p, Pxa2p, Rli1p, Snq2p, Step 6p, Vma8p, Vmr1p, Ybt1p, Ycf1p, Yor1p, YKR104W, YOL075C, Aqr1p, Atr1p, Azr1p, Dtr1p, Enb1p, Flr1p, Hol1p, Pdr8p, Qdr1p, Qdr2p, Qdr3p, Seo1p, Sge1p, Ssu1p, Thi7p, Tpn1p, Vba5p, YIL166C, Agp1p, Agp2p, Agp3p, Alp1p, Bap2p, Bap3p, Bio5p, Can1p, Dip5p, Gap1p, Gnp1p, Hip1p, Hnm1p, Lyp1p, Mmp1p, Put4p, Sam3p, Ssy1p, Tat1p, Tat2p, Tpo1p, Tpo2p, Tpo3p, Tpo4p, Tpo5p, and Uga4p. In a further embodiment, the recombinant microorganism comprising a biosynthetic pathway which uses acetolactate as an intermediate may be engineered to reduce the activity or expression of an endogenous transcriptional regulator of an endogenous transporter protein. In an exemplary embodiment, the transcriptional regulator is War1p

Use of Overexpressed Ketol-Acid Reductoisomerase (KARI) and/or Modified Ketol-Acid Reductoisomerase (KARI) to Reduce the Production of 2,3-Butanediol

As described herein, the conversion of acetolactate to 2,3-butanediol competes with the isobutanol pathway for the intermediate acetolactate. In many yeast isobutanol production strains, the conversion of acetolactate to DHIV is catalyzed by the enzyme ketol-acid reductoisomerase (KARI).

In one embodiment, the present invention provides recombinant microorganisms having an overexpressed ketol-acid reductoisomerase (KARI). The overexpression of KARI has the effect of reducing 2,3-butanediol production. In one embodiment, the KARI has at least 0.01 U/mg of activity in the lysate. In another embodiment, the KARI has at least 0.03 U/mg of activity in the lysate. In yet another embodiment, the KARI has at least 0.05, 0.1, 0.5, 1, 2, 5, or 10 U/mg of activity in the lysate.

In a preferred embodiment, the overexpressed KARI is engineered to exhibit a reduced K_{M} for acetolactate as compared to a wild-type or parental KARI. The use of the modified KARI with lower K_{M} for acetolactate is expected to reduce the production of the by-product 2,3-butanediol. A KARI with lower substrate K_{M} is identified by screening homologs. In the alternative, the KARI can be engineered to exhibit reduced K_{M} by directed evolution using techniques known in the art.

In each of these embodiments, the KARI may be a variant enzyme that utilizes NADH (rather than NADPH) as a co-factor. Such enzymes are described in the commonly owned and co-pending publication, US 2010/0143997, which is herein incorporated by reference in its entirety for all purposes.

### Use of Overexpressed Dihydroxy Acid Dehydratase (DHAD) to Reduce the Production of 2,3-Butanediol

As described herein, the present inventors have found that overexpression of the isobutanol pathway enzyme, dihydroxyacid dehydratase (DHAD), reduces the production of the by-product, 2,3-butanediol.

Accordingly, in one embodiment, the present invention provides recombinant microorganisms having an overexpressed dihydroxyacid dehydratase (DHAD), which catalyzes the conversion of 2,3-dihydroxyisovalerate (DHIV) to 2-ketoisovalerate (KIV). The overexpression of DHAD has the effect of reducing 2,3-butanediol production. In one embodiment, the DHAD has at least 0.01 U/mg of activity in the lysate. In another embodiment, the DHAD has at least 0.03 U/mg of activity in the lysate. In yet another embodiment, the DHAD has at least 0.05, 0.1, 0.5, 1, 2, 5, or 10 U/mg of activity in the lysate.

### Recombinant Microorganisms for the Production of Acetoin

The present invention provides in additional aspects recombinant microorganisms for the production of acetoin as a product or a metabolic intermediate. In one embodiment, these acetoin-producing recombinant microorganisms express acetolactate synthase (ALS) and an acetolactate decarboxylase catalyzing the decarboxylation of acetolactate to acetoin. In one embodiment, the acetolactate decarboxylase is overexpressed. In another embodiment, the acetoin-producing recombinant microorganisms of the present invention express acetolactate synthase (ALS) and a diacetyl reductase catalyzing the conversion of diacetyl to acetoin. In one embodiment, the diacetyl reductase is overexpressed.

These acetoin-producing recombinant microorganisms may be further engineered to reduce or eliminate enzymatic activity for the conversion of pyruvate to products other than acetolactate. In one embodiment, the enzymatic activity of pyruvate decarboxylase (PDC), lactate dehydrogenase (LDH), pyruvate oxidase, pyruvate dehydrogenase, and/or glycerol-3-phosphate dehydrogenase (GPD) is reduced or eliminated.

In a specific embodiment, acetoin is produced in a recombinant PDC-minus GPD-minus yeast microorganism that overexpresses an ALS gene and expresses an acetolactate decarboxylase. In one embodiment, the acetolactate decarboxylase is natively expressed. In another embodiment, the acetolactate decarboxylase is heterologously expressed. In yet another embodiment, the acetolactate decarboxylase is overexpressed. In a specific embodiment, the acetolactate decarboxylase is encoded by the S. cerevisiae acetolactate decarboxylase or a homolog thereof.

In another specific embodiment, acetoin is produced in a recombinant PDC-minus GPD-minus yeast microorganism that overexpresses an ALS gene and expresses a diacetyl reductase. In one embodiment, the diacetyl reductase is natively expressed. In another embodiment, the diacetyl reductase is heterologously expressed. In yet another embodiment, the diacetyl reductase is overexpressed. In a specific embodiment, the diacetyl reductase is encoded by the S. cerevisiae OYE2 (SEQ ID NO: 1) gene or a homolog thereof. In another specific embodiment, the diacetyl reductase is encoded by the S. cerevisiae ARA1 (SEQ ID NO: 3) gene or a homolog thereof. In yet another specific embodiment, the diacetyl reductase is encoded by one of the S. cerevisiae genes, BDH1 (SEQ ID NO: 5), BDH2 (SEQ ID NO: 7), ERG19 (SEQ ID NO: 9), GCY1 (SEQ ID NO: 11), GRE3 (SEQ ID NO: 13), OYE3 (SEQ ID NO: 15), TRR1 (SEQ ID NO: 17), YPR1 (SEQ ID NO: 19), ZWF1 (SEQ ID NO: 21), and YPL088W (SEQ ID NO: 23), or homologs or variants thereof.

In accordance with these additional aspects, the present invention also provides a method of producing acetoin, comprising: (a) providing an acetoin-producing recombinant microorganism that expresses acetolactate synthase (ALS) and an acetolactate decarboxylase catalyzing the decarboxylation of acetolactate to acetoin, and (b) cultivating said recombinant microorganism in a culture medium containing a feedstock providing the carbon source, until a recoverable quantity of acetoin is produced.

In accordance with these additional aspects, the present invention also provides a method of producing acetoin, comprising: (a) providing an acetoin-producing recombinant microorganism that expresses acetolactate synthase (ALS) and a diacetyl reductase catalyzing the conversion of diacetyl to acetoin, and (b) cultivating said recombinant microorganism in a culture medium containing a feedstock providing the carbon source, until a recoverable quantity of acetoin is produced.

### Recombinant Microorganisms for the Production of 2,3-Butanediol

The present invention provides in additional aspects recombinant microorganisms for the production of 2,3-butanediol as a product or a metabolic intermediate. In one embodiment, these 2,3-butanediol-producing recombinant microorganisms express acetolactate synthase (ALS) and an acetoin reductase catalyzing the conversion of acetoin to 2,3-butanediol. In one embodiment, the acetoin reductase is overexpressed. In another embodiment, the recombinant microorganism further overexpresses an acetolactate decarboxylase or diacetyl reductase.

These 2,3-butanediol-producing recombinant microorganisms may be further engineered to reduce or eliminate enzymatic activity for the conversion of pyruvate to products other than acetolactate. In one embodiment, the enzymatic activity of pyruvate decarboxylase (PDC), lactate dehydrogenase (LDH), pyruvate oxidase, pyruvate dehydrogenase, and/or glycerol-3-phosphate dehydrogenase (GPD) is reduced or eliminated.

In a specific embodiment, 2,3-butanediol is produced in a recombinant PDC-minus GPD-minus yeast microorganism that overexpresses an ALS gene and expresses an acetoin reductase. In one embodiment, the acetoin reductase is natively expressed. In another embodiment, the acetoin reductase is heterologously expressed. In yet another embodiment, the acetoin reductase is overexpressed. In a specific embodiment, the acetoin reductase is encoded by a gene selected from BDH1, BDH2, ARA1, ERG19, GRE3, OYE2, OYE3, TRR1, YPR1, ZWF1, and YPL088W, or homologs or variants thereof. In another embodiment, the recombinant microorganism further overexpresses an acetolactate decarboxylase and/or a diacetyl reductase.

In accordance with these additional aspects, the present invention also provides a method of producing 2,3-butanediol, comprising: (a) providing a 2,3-butanediol-producing recombinant microorganism that expresses acetolactate synthase (ALS) and an acetoin reductase catalyzing the conversion of acetoin to 2,3-butanediol, and (b) cultivating said recombinant microorganism in a culture medium containing a feedstock providing the carbon source, until a recoverable quantity of 2,3-butanediol is produced. In some embodiments, the recombinant microorganism may further overexpress an acetolactate decarboxylase and/or a diacetyl reductase.

### Isobutanol-Producing Yeast Microorganisms

In certain exemplary embodiments, the present application relates to a recombinant yeast microorganism comprising an engineered isobutanol producing metabolic pathway. In recent years, yeast cells have been engineered to produce increased quantities of isobutanol, an important commodity chemical and biofuel candidate (See, e.g., commonly owned and co-pending patent publications, US 2009/0226991, US 2010/0143997, US 2011/0020889, US 2011/0076733, US 2011/0201090, and WO 2010/075504).

As described herein, the present invention relates to recombinant microorganisms for producing isobutanol, wherein said recombinant microorganisms comprise an isobutanol producing metabolic pathway. In one embodiment, the isobutanol producing metabolic pathway to convert pyruvate to isobutanol can be comprised of the following reactions:

1. 2 pyruvate→acetolactate+CO₂

2. acetolactate+NAD(P)H→2,3-dihydroxyisovalerate+NAD(P)⁺

3. 2,3-dihydroxyisovalerate→alpha-ketoisovalerate

4. alpha-ketoisovalerate→isobutyraldehyde+CO₂

5. isobutyraldehyde+NAD(P)H→isobutanol+NADP

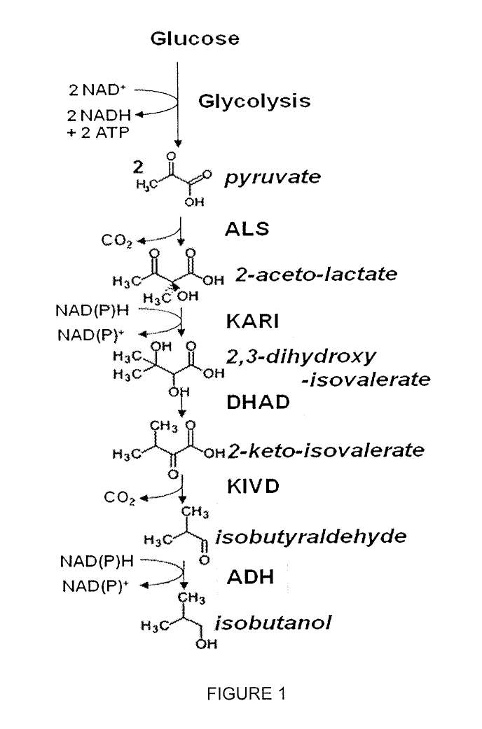

In one embodiment, these reactions are carried out by the enzymes 1) Acetolactate synthase (ALS), 2) Ketol-acid reductoisomerase (KARI), 3) Dihydroxy-acid dehydratase (DHAD), 4) 2-keto-acid decarboxylase, e.g., Keto-isovalerate decarboxylase (KIVD), and 5) an Alcohol dehydrogenase (ADH) (FIG. 1). In some embodiments, the recombinant microorganism may be engineered to overexpress one or more of these enzymes. In an exemplary embodiment, the recombinant microorganism is engineered to overexpress all of these enzymes.

Alternative pathways for the production of isobutanol in yeast have been described in WO/2007/050671 and in Dickinson et al., 1998, J Biol Chem 273:25751-6. These and other isobutanol producing metabolic pathways are within the scope of the present application. In one embodiment, the isobutanol producing metabolic pathway comprises five substrate to product reactions. In another embodiment, the isobutanol producing metabolic pathway comprises six substrate to product reactions. In yet another embodiment, the isobutanol producing metabolic pathway comprises seven substrate to product reactions.

In various embodiments described herein, the recombinant microorganism comprises an engineered isobutanol producing metabolic pathway. In one embodiment, the isobutanol producing metabolic pathway comprises at least one exogenous gene encoding a polypeptide that catalyzes a step in the conversion of pyruvate to isobutanol. In another embodiment, the isobutanol producing metabolic pathway comprises at least two exogenous genes encoding polypeptides that catalyze steps in the conversion of pyruvate to isobutanol. In yet another embodiment, the isobutanol producing metabolic pathway comprises at least three exogenous genes encoding polypeptides that catalyze steps in the conversion of pyruvate to isobutanol. In yet another embodiment, the isobutanol producing metabolic pathway comprises at least four exogenous genes encoding polypeptides that catalyze steps in the conversion of pyruvate to isobutanol. In yet another embodiment, the isobutanol producing metabolic pathway comprises at least five exogenous genes encoding polypeptides that catalyze steps in the conversion of pyruvate to isobutanol. In yet another embodiment, all of the isobutanol producing metabolic pathway steps in the conversion of pyruvate to isobutanol are converted by exogenously encoded enzymes.

In one embodiment, one or more of the isobutanol pathway genes encodes an enzyme that is localized to the cytosol. In one embodiment, the recombinant microorganisms comprise an isobutanol producing metabolic pathway with at least one isobutanol pathway enzyme localized in the cytosol. In another embodiment, the recombinant microorganisms comprise an isobutanol producing metabolic pathway with at least two isobutanol pathway enzymes localized in the cytosol. In yet another embodiment, the recombinant microorganisms comprise an isobutanol producing metabolic pathway with at least three isobutanol pathway enzymes localized in the cytosol. In yet another embodiment, the recombinant microorganisms comprise an isobutanol producing metabolic pathway with at least four isobutanol pathway enzymes localized in the cytosol. In an exemplary embodiment, the recombinant microorganisms comprise an isobutanol producing metabolic pathway with five isobutanol pathway enzymes localized in the cytosol. Isobutanol producing metabolic pathways in which one or more genes are localized to the cytosol are described in commonly owned and co-pending publication, US 2011/0076733, which is herein incorporated by reference in its entirety for all purposes.

As is understood in the art, a variety of organisms can serve as sources for the isobutanol pathway enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but not limited to, Piromyces spp., Orpinomyces spp., or Neocallimastix spp. Sources of prokaryotic enzymes that are useful include, but not limited to, Escherichia spp., Zymomonas spp., Staphylococcus spp., Bacillus spp., Clostridium spp., Corynebacterium spp., Pseudomonas spp., Slackia spp., Lactococcus spp., Enterobacter spp., Streptococcus spp., Salmonella spp., Bacteroides spp., Methanococcus spp., Erythrobacter spp., Sphingomonas spp., Sphingobium spp., and Novosphingobium spp.

In some embodiments, one or more of these enzymes can be encoded by native genes. Alternatively, one or more of these enzymes can be encoded by heterologous genes. For example, ALS can be encoded by the alsS gene of B. subtilis, alsS of L. lactis, or the ilvK gene of K. pneumonia. For example, KARI can be encoded by the ilvC gene of E. coli, L. lactis, S. exigua, S. enterica, or Shewanella sp, or variants of said genes which have been engineered to encode NADH-dependent KARIs (“NKRs”). For example, DHAD can be encoded by the ilvD gene of E. coli, C. glutamicum, L. lactis, or S. mutans. For example, KIVD can be encoded by the kivD or kdcA gene of L. lactis. For example, ADH can be encoded by ADH2, ADH6, or ADH7 of S. cerevisiae, the adhA gene of L. lactis, or an alcohol dehydrogenase gene from D. melanogaster. A representative listing of genes encoding functional enzymes for each of the five pathway steps are disclosed in commonly owned and co-pending patent publications, US 2009/0226991, US 2010/0143997, US 2011/0020889, US 2011/0076733, US 2011/0201090, and WO 2010/075504, each of which is herein incorporated by reference in its entirety).

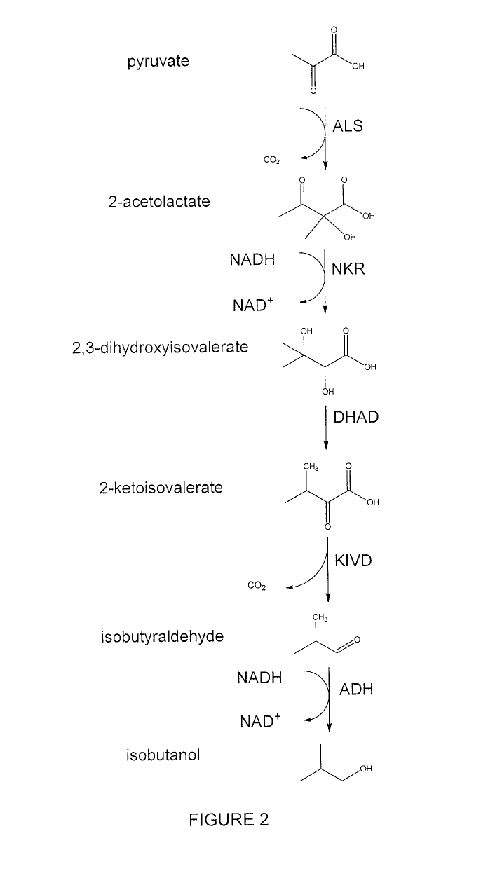

In an exemplary embodiment, pathway steps 2 and 5 of the isobutanol pathway may be carried out by KARI and ADH enzymes that utilize NADH (rather than NADPH) as a cofactor. It has been found previously that utilization of NADH-dependent KARI (NKR) and ADH enzymes to catalyze pathway steps 2 and 5, respectively, surprisingly enables production of isobutanol at theoretical yield and/or under anaerobic conditions. See, e.g., commonly owned and co-pending patent publication US 2010/0143997. An example of an NADH-dependent isobutanol pathway is illustrated in FIG. 2. Thus, in one embodiment, the recombinant microorganisms of the present invention may use an NKR to catalyze the conversion of acetolactate to produce 2,3-dihydroxyisovalerate. In another embodiment, the recombinant microorganisms of the present invention may use an NADH-dependent ADH to catalyze the conversion of isobutyraldehyde to produce isobutanol. In yet another embodiment, the recombinant microorganisms of the present invention may use both an NKR to catalyze the conversion of acetolactate to produce 2,3-dihydroxyisovalerate, and an NADH-dependent ADH to catalyze the conversion of isobutyraldehyde to produce isobutanol.

In another embodiment, the yeast microorganism may be engineered to have increased ability to convert pyruvate to isobutanol. In one embodiment, the yeast microorganism may be engineered to have increased ability to convert pyruvate to isobutyraldehyde. In another embodiment, the yeast microorganism may be engineered to have increased ability to convert pyruvate to keto-isovalerate. In another embodiment, the yeast microorganism may be engineered to have increased ability to convert pyruvate to 2,3-dihydroxyisovalerate. In another embodiment, the yeast microorganism may be engineered to have increased ability to convert pyruvate to acetolactate.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

In one embodiment, the invention is directed to a recombinant microorganism for producing isobutanol, wherein said recombinant microorganism comprises an isobutanol producing metabolic pathway and wherein said microorganism is engineered to reduce or eliminate the expression or activity of an enzyme catalyzing the conversion of acetolactate to 2,3-butanediol. In one embodiment, the recombinant microorganism is engineered to reduce or eliminate the expression or activity of an enzyme catalyzing the conversion of acetolactate to acetoin. In another embodiment, the recombinant microorganism is engineered to reduce or eliminate the expression or activity of an enzyme catalyzing the conversion of diacetyl to acetoin. In yet another embodiment, the recombinant microorganism is engineered to reduce or eliminate the expression or activity of an enzyme catalyzing the conversion of acetoin to 2,3-butanediol.

In some embodiments, the enzyme catalyzing the conversion of acetolactate to 2,3-butanediol is an acetolactate decarboxylase. In an exemplary embodiment, the acetolactate decarboxylase (ALDC) is the S. cerevisiae acetolactate decarboxylase or a homolog or variant thereof. Accordingly, in one embodiment, the invention is directed to a recombinant microorganism for producing isobutanol, wherein said recombinant microorganism comprises an isobutanol producing metabolic pathway and wherein said microorganism is engineered to reduce or eliminate the expression or activity of one or more acetolactate decarboxylases.

In some embodiments, the enzyme catalyzing the conversion of diacetyl to acetoin is a diacetyl reductase. In an exemplary embodiment, the diacetyl reductase is the S. cerevisiae protein, Oye2p, or a homolog or variant thereof. In another exemplary embodiment, the diacetyl reductase is the S. cerevisiae protein, Ara1p, or a homolog or variant thereof. In yet another exemplary embodiment, the diacetyl reductase is selected from the S. cerevisiae proteins Bdh1p, Bdh2p, Erg19p, Gcy1p, Gre3p, Oye3p, Trr1p, Ypr1p, Zwf1p, and YPL088W, or homologs or variants thereof. Accordingly, in one embodiment, the invention is directed to a recombinant microorganism for producing isobutanol, wherein said recombinant microorganism comprises an isobutanol producing metabolic pathway and wherein said microorganism is engineered to reduce or eliminate the expression or activity of one or more diacetyl red uctases.

In some embodiments, the enzyme catalyzing the conversion of acetoin to 2,3-butanediol is an acetoin reductase. In an exemplary embodiment, the acetoin reductase is the S. cerevisiae protein, Bdh1p, or a homolog or variant thereof. In another exemplary embodiment, acetoin reductase is the S. cerevisiae protein, Bdh2p, or a homolog or variant thereof. In yet another exemplary embodiment, the acetoin reductase is the S. cerevisiae protein, Ara1p, or a homolog or variant thereof. In yet another exemplary embodiment, the acetoin reductase is selected from the S. cerevisiae proteins Erg 19p, Gcy1p, Gre3p, Oye2p, Oye3p, Trr1p, Ypr1p, Zwf1p, and YPL088W, or homologs or variants thereof. Accordingly, in one embodiment, the invention is directed to a recombinant microorganism for producing isobutanol, wherein said recombinant microorganism comprises an isobutanol producing metabolic pathway and wherein said microorganism is engineered to reduce or eliminate the expression or activity of one or more acetoin reductases.

In yet another embodiment, the invention is directed to a recombinant microorganism for producing isobutanol, wherein said recombinant microorganism comprises an isobutanol producing metabolic pathway and wherein said microorganism is engineered to reduce or eliminate the expression or activity of at least two of the following: (i) one or more enzymes catalyzing the conversion of acetolactate to acetoin; (ii) one or more enzymes catalyzing the conversion of diacetyl to acetoin; and/or (iii) one or more enzymes catalyzing the conversion of acetoin to 2,3-butanediol.

In another embodiment, the invention is directed to a recombinant microorganism for producing isobutanol, wherein said recombinant microorganism comprises an isobutanol producing metabolic pathway and wherein said microorganism is engineered to reduce or eliminate the expression or activity of all of the following: (i) one or more enzymes catalyzing the conversion of acetolactate to acetoin; (ii) one or more enzymes catalyzing the conversion of diacetyl to acetoin; and (iii) one or more enzymes catalyzing the conversion of acetoin to 2,3-butanediol.

### The Microorganism in General

As described herein, the recombinant microorganisms of the present invention can express a plurality of heterologous and/or native enzymes involved in pathways for the production of a desired metabolite (e.g., a commodity chemical such as isobutanol).

As described herein, “engineered” or “modified” microorganisms are produced via the introduction of genetic material into a host or parental microorganism of choice and/or by modification of the expression of native genes, thereby modifying or altering the cellular physiology and biochemistry of the microorganism. Through the introduction of genetic material and/or the modification of the expression of native genes the parental microorganism acquires new properties, e.g., the ability to produce a new, or greater quantities of, an intracellular and/or extracellular metabolite. As described herein, the introduction of genetic material into and/or the modification of the expression of native genes in a parental microorganism results in a new or modified ability to produce a desired metabolite (e.g., a commodity chemical such as isobutanol) from a suitable carbon source. The genetic material introduced into and/or the genes modified for expression in the parental microorganism contains gene(s), or parts of genes, coding for one or more of the enzymes involved in a biosynthetic pathway for the production of a desired metabolite (e.g., a commodity chemical such as isobutanol) and may also include additional elements for the expression and/or regulation of expression of these genes, e.g., promoter sequences.

In addition to the introduction of a genetic material into a host or parental microorganism, an engineered or modified microorganism can also include the alteration, disruption, deletion or knocking-out of a gene or polynucleotide to alter the cellular physiology and biochemistry of the microorganism. Through the alteration, disruption, deletion or knocking-out of a gene or polynucleotide, the microorganism acquires new or improved properties (e.g., the ability to produce a new metabolite or greater quantities of an intracellular metabolite, to improve the flux of a metabolite down a desired pathway, and/or to reduce the production of by-products).

Recombinant microorganisms provided herein may also produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., 2-ketoisovalerate), or an end product (e.g., a higher alcohol such as isobutanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating a desired metabolite (e.g., a commodity chemical such as isobutanol) are encompassed by the microorganisms and methods provided herein.

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25: 365-89).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See commonly owned U.S. Pat. No. 8,017,375. A typical algorithm used comparing a molecule sequence to a database containing a large number of sequences from different organisms is the computer program BLAST. When searching a database containing sequences from a large number of different organisms, it is typical to compare amino acid sequences. Database searching using amino acid sequences can be measured by algorithms described in commonly owned U.S. Pat. No. 8,017,375.

It is understood that a range of microorganisms can be modified to include a recombinant metabolic pathway suitable for the production of a desired metabolite (e.g., a commodity chemical such as isobutanol). In various embodiments, microorganisms may be selected from yeast microorganisms. Yeast microorganisms for the production of a desired metabolite (e.g., a commodity chemical such as isobutanol) may be selected based on certain characteristics:

One characteristic may include the property that the microorganism is selected to convert various carbon sources into a desired metabolite (e.g., a commodity chemical such as isobutanol). The term “carbon source” generally refers to a substance suitable to be used as a source of carbon for prokaryotic or eukaryotic cell growth. Examples of suitable carbon sources are described in commonly owned U.S. Pat. No. 8,017,375. Accordingly, in one embodiment, the recombinant microorganism herein disclosed can convert a variety of carbon sources to products, including but not limited to glucose, galactose, mannose, xylose, arabinose, lactose, sucrose, and mixtures thereof.

The recombinant microorganism may thus further include a pathway for the production of a desired metabolite (e.g., a commodity chemical such as isobutanol) from five-carbon (pentose) sugars including xylose. Most yeast species metabolize xylose via a complex route, in which xylose is first reduced to xylitol via a xylose reductase (XR) enzyme. The xylitol is then oxidized to xylulose via a xylitol dehydrogenase (XDH) enzyme. The xylulose is then phosphorylated via a xylulokinase (XK) enzyme. This pathway operates inefficiently in yeast species because it introduces a redox imbalance in the cell. The xylose-to-xylitol step uses primarily NADPH as a cofactor (generating NADP+), whereas the xylitol-to-xylulose step uses NAD+ as a cofactor (generating NADH). Other processes must operate to restore the redox imbalance within the cell. This often means that the organism cannot grow anaerobically on xylose or other pentose sugars. Accordingly, a yeast species that can efficiently ferment xylose and other pentose sugars into a desired fermentation product is therefore very desirable.

Thus, in one aspect, the recombinant microorganism is engineered to express a functional exogenous xylose isomerase. Exogenous xylose isomerases (XI) functional in yeast are known in the art. See, e.g., Rajgarhia et al., U.S. Pat. No. 7,943,366, which is herein incorporated by reference in its entirety. In an embodiment according to this aspect, the exogenous XI gene is operatively linked to promoter and terminator sequences that are functional in the yeast cell. In a preferred embodiment, the recombinant microorganism further has a deletion or disruption of a native gene that encodes for an enzyme (e.g., XR and/or XDH) that catalyzes the conversion of xylose to xylitol. In a further preferred embodiment, the recombinant microorganism also contains a functional, exogenous xylulokinase (XK) gene operatively linked to promoter and terminator sequences that are functional in the yeast cell. In one embodiment, the xylulokinase (XK) gene is overexpressed.

In one embodiment, the microorganism has reduced or no pyruvate decarboxylase (PDC) activity. PDC catalyzes the decarboxylation of pyruvate to acetaldehyde, which is then reduced to ethanol by ADH via an oxidation of NADH to NAD+. Ethanol production is the main pathway to oxidize the NADH from glycolysis. Deletion of this pathway increases the pyruvate and the reducing equivalents (NADH) available for the isobutanol producing metabolic pathway. Accordingly, deletion of genes encoding for pyruvate decarboxylases can further increase the yield of the desired metabolite (e.g., a commodity chemical such as isobutanol).

In another embodiment, the microorganism has reduced or no glycerol-3-phosphate dehydrogenase (GPD) activity. GPD catalyzes the reduction of dihydroxyacetone phosphate (DHAP) to glycerol-3-phosphate (G3P) via the oxidation of NADH to NAD+. Glycerol is then produced from G3P by Glycerol-3-phosphatase (GPP). Glycerol production is a secondary pathway to oxidize excess NADH from glycolysis. Reduction or elimination of this pathway would increase the pyruvate and reducing equivalents (NADH) available for the isobutanol producing metabolic pathway. Thus, deletion of genes encoding for glycerol-3-phosphate dehydrogenases can further increase the yield of the desired metabolite (e.g., a commodity chemical such as isobutanol).

In yet another embodiment, the microorganism has reduced or no 3-keto acid reductase (3-KAR) activity. 3-keto acid reductase catalyzes the conversion of 3-keto acids (e.g., acetolactate) to 3-hydroxyacids (e.g., DH2 MB). 3-KAR-minus yeast production strains are described in commonly owned and co-pending U.S. Publication No. 2011/0201090, which is herein incorporated by reference in its entirety for all purposes.

In yet another embodiment, the microorganism has reduced or no aldehyde dehydrogenase (ALDH) activity. Aldehyde dehydrogenases catalyze the conversion of aldehydes (e.g., isobutyraldehyde) to acid by-products (e.g., isobutyrate). ALDH-minus yeast production strains are described in commonly owned and co-pending U.S. Publication No. 2011/0201090, which is herein incorporated by reference in its entirety for all purposes.

In one embodiment, the yeast microorganisms may be selected from the “Saccharomyces Yeast Clade”, as described in commonly owned U.S. Pat. No. 8,017,375.

The term “Saccharomyces sensu stricto” taxonomy group is a cluster of yeast species that are highly related to S. cerevisiae (Rainieri et al., 2003, J. Biosci Bioengin 96: 1-9). Saccharomyces sensu stricto yeast species include but are not limited to S. cerevisiae, S. kudriavzevii, S. mikatae, S. bayanus, S. uvarum, S. carocanis and hybrids derived from these species (Masneuf et al., 1998, Yeast 7: 61-72).

An ancient whole genome duplication (WGD) event occurred during the evolution of the hemiascomycete yeast and was discovered using comparative genomic tools (Kellis et al., 2004, Nature 428: 617-24; Dujon et al., 2004, Nature 430:35-44; Langkjaer et al., 2003, Nature 428: 848-52; Wolfe et al., 1997, Nature 387: 708-13). Using this major evolutionary event, yeast can be divided into species that diverged from a common ancestor following the WGD event (termed “post-WGD yeast” herein) and species that diverged from the yeast lineage prior to the WGD event (termed “pre-WGD yeast” herein).

Accordingly, in one embodiment, the yeast microorganism may be selected from a post-WGD yeast genus, including but not limited to Saccharomyces and Candida. The favored post-WGD yeast species include: S. cerevisiae, S. uvarum, S. bayanus, S. paradoxus, S. castelli, and C. glabrata.

In another embodiment, the yeast microorganism may be selected from a pre-whole genome duplication (pre-WGD) yeast genus including but not limited to Saccharomyces, Kluyveromyces, Candida, Pichia, Issatchenkia, Debaryomyces, Hansenula, Yarrowia and, Schizosaccharomyces. Representative pre-WGD yeast species include: S. kluyveri, K. thermotolerans, K. marxianus, K. waltii, K. lactis, C. tropicalis, P. pastoris, P. anomala, P. stipitis, I. orientalis, I. occidentalis, I. scutulata, D. hansenii, H. anomala, Y. lipolytica, and S. pombe.

A yeast microorganism may be either Crabtree-negative or Crabtree-positive as described in described in commonly owned U.S. Pat. No. 8,017,375. In one embodiment the yeast microorganism may be selected from yeast with a Crabtree-negative phenotype including but not limited to the following genera: Saccharomyces, Kluyveromyces, Pichia, Issatchenkia, Hansenula, and Candida. Crabtree-negative species include but are not limited to: S. kluyveri, K. lactis, K. marxianus, P. anomala, P. stipitis, I. orientalis, I. occidentalis, I. scutulata, H. anomala, and C. utills. In another embodiment, the yeast microorganism may be selected from yeast with a Crabtree-positive phenotype, including but not limited to Saccharomyces, Kluyveromyces, Zygosaccharomyces, Debaryomyces, Pichia and Schizosaccharomyces. Crabtree-positive yeast species include but are not limited to: S. cerevisiae, S. uvarum, S. bayanus, S. paradoxus, S. castelli, K. thermotolerans, C. glabrata, Z. bailli, Z. rouxii, D. hansenii, P. pastorius, and S. pombe.

Another characteristic may include the property that the microorganism is that it is non-fermenting. In other words, it cannot metabolize a carbon source anaerobically while the yeast is able to metabolize a carbon source in the presence of oxygen. Nonfermenting yeast refers to both naturally occurring yeasts as well as genetically modified yeast. During anaerobic fermentation with fermentative yeast, the main pathway to oxidize the NADH from glycolysis is through the production of ethanol. Ethanol is produced by alcohol dehydrogenase (ADH) via the reduction of acetaldehyde, which is generated from pyruvate by pyruvate decarboxylase (PDC). In one embodiment, a fermentative yeast can be engineered to be non-fermentative by the reduction or elimination of the native PDC activity. Thus, most of the pyruvate produced by glycolysis is not consumed by PDC and is available for the isobutanol pathway. Deletion of this pathway increases the pyruvate and the reducing equivalents available for the biosynthetic pathway. Fermentative pathways contribute to low yield and low productivity of higher alcohols such as isobutanol. Accordingly, deletion of one or more PDC genes may increase yield and productivity of a desired metabolite (e.g., a commodity chemical such as isobutanol).

In some embodiments, the recombinant microorganisms may be microorganisms that are non-fermenting yeast microorganisms, including, but not limited to those, classified into a genera selected from the group consisting of Tricosporon, Rhodotorula, Myxozyma, or Candida. In a specific embodiment, the non-fermenting yeast is C. xestobii.

### Methods in General

### Identification of Homologs of Acetolactate Decarboxylases, Diacetyl Reductases, and Acetoin Reductases

Any method can be used to identify genes that encode for enzymes that are homologous to the genes described herein (e.g., acetolactate decarboxylase homologs, diacetyl reductase homologs, and acetoin reductase homologs). Generally, genes that are homologous or similar to the acetolactate decarboxylases, diacetyl reductases, and acetoin reductases described herein may be identified by functional, structural, and/or genetic analysis. In most cases, homologous or similar genes and/or homologous or similar enzymes will have functional, structural, or genetic similarities.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous genes, proteins, or enzymes, techniques may include, but not limited to, cloning a gene by PCR using primers based on a published sequence of a gene/enzyme or by degenerate PCR using degenerate primers designed to amplify a conserved region among dehydratase genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K. Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein.

### Identification of PDC and GPD in a Yeast Microorganism

Any method can be used to identify genes that encode for enzymes with pyruvate decarboxylase (PDC) activity or glycerol-3-phosphate dehydrogenase (GPD) activity. Suitable methods for the identification of pyruvate decarboxylases and glycerol-3-phosphate dehydrogenases are described in commonly owned U.S. Pat. No. 8,017,375 and U.S. Publication No. 2011/0020889, both of which are herein incorporated by reference in their entireties for all purposes.

### Identification of 3-Keto Acid Reductases and Aldehyde Dehydrogenases in a Yeast Microorganism

Any method can be used to identify genes that encode for enzymes with 3-keto acid reductase (3-KAR) activity or aldehyde dehydrogenase (ALDH) activity. Suitable methods for the identification of 3-keto acid reductases and aldehyde dehydrogenases are described in commonly owned and co-pending U.S. Publication No. 2011/0201090, which is herein incorporated by reference in its entirety for all purposes.

### Genetic Insertions and Deletions

Any method can be used to introduce a nucleic acid molecule into yeast and many such methods are well known. For example, transformation and electroporation are common methods for introducing nucleic acid into yeast cells. See, e.g., Gietz et al., 1992, Nuc Acids Res. 27: 69-74; Ito et al., 1983, J. Bacteriol. 153: 163-8; and Becker et al., 1991, Methods in Enzymology 194: 182-7.

In an embodiment, the integration of a gene of interest into a DNA fragment or target gene of a yeast microorganism occurs according to the principle of homologous recombination. According to this embodiment, an integration cassette containing a module comprising at least one yeast marker gene and/or the gene to be integrated (internal module) is flanked on either side by DNA fragments homologous to those of the ends of the targeted integration site (recombinogenic sequences). After transforming the yeast with the cassette by appropriate methods, a homologous recombination between the recombinogenic sequences may result in the internal module replacing the chromosomal region in between the two sites of the genome corresponding to the recombinogenic sequences of the integration cassette. (Orr-Weaver et al., 1981, PNAS USA 78: 6354-58).

In an embodiment, the integration cassette for integration of a gene of interest into a yeast microorganism includes the heterologous gene under the control of an appropriate promoter and terminator together with the selectable marker flanked by recombinogenic sequences for integration of a heterologous gene into the yeast chromosome. In an embodiment, the heterologous gene includes an appropriate native gene desired to increase the copy number of a native gene(s). The selectable marker gene can be any marker gene used in yeast, including but not limited to, H1S3, TRP1, LEU2, URA3, bar, ble, hph, and kan. The recombinogenic sequences can be chosen at will, depending on the desired integration site suitable for the desired application.

In another embodiment, integration of a gene into the chromosome of the yeast microorganism may occur via random integration (Kooistra et al., 2004, Yeast 21: 781-792).

Additionally, in an embodiment, certain introduced marker genes are removed from the genome using techniques well known to those skilled in the art. For example, URA3 marker loss can be obtained by plating URA3 containing cells in FOA (5-fluoro-orotic acid) containing medium and selecting for FOA resistant colonies (Boeke et al., 1984, Mol. Gen. Genet. 197: 345-47).

The exogenous nucleic acid molecule contained within a yeast cell of the disclosure can be maintained within that cell in any form. For example, exogenous nucleic acid molecules can be integrated into the genome of the cell or maintained in an episomal state that can stably be passed on (“inherited”) to daughter cells. Such extra-chromosomal genetic elements (such as plasmids, mitochondrial genome, etc.) can additionally contain selection markers that ensure the presence of such genetic elements in daughter cells. Moreover, the yeast cells can be stably or transiently transformed. In addition, the yeast cells described herein can contain a single copy, or multiple copies of a particular exogenous nucleic acid molecule as described above.

### Reduction of Enzymatic Activity

Yeast microorganisms within the scope of the invention may have reduced enzymatic activity such as reduced acetolactate decarboxylase, diacetyl reductase, acetoin reductase, 3-KAR, ALDH, PDC, or GPD activity. The term “reduced” as used herein with respect to a particular enzymatic activity refers to a lower level of enzymatic activity than that measured in a comparable yeast cell of the same species. The term reduced also refers to the elimination of enzymatic activity as compared to a comparable yeast cell of the same species. Thus, yeast cells lacking acetolactate decarboxylase, diacetyl reductase, acetoin reductase, 3-KAR, ALDH, PDC, or GPD activity are considered to have reduced acetolactate decarboxylase, diacetyl reductase, acetoin reductase, 3-KAR, ALDH, PDC, or GPD activity since most, if not all, comparable yeast strains have at least some acetolactate decarboxylase, diacetyl reductase, acetoin reductase, 3-KAR, ALDH, PDC, or GPD activity. Such reduced enzymatic activities can be the result of lower enzyme concentration, lower specific activity of an enzyme, or a combination thereof. Many different methods can be used to make yeast having reduced enzymatic activity. For example, a yeast cell can be engineered to have a disrupted enzyme-encoding locus using common mutagenesis or knock-out technology. See, e.g., Methods in Yeast Genetics (1997 edition), Adams, Gottschling, Kaiser, and Stems, Cold Spring Harbor Press (1998). In addition, certain point-mutation(s) can be introduced which results in an enzyme with reduced activity. Also included within the scope of this invention are yeast strains which when found in nature, are substantially free of one or more activities selected from acetolactate decarboxylase, diacetyl reductase, acetoin reductase, 3-KAR, ALDH, PDC, or GPD activity.

Alternatively, antisense technology can be used to reduce enzymatic activity. For example, yeast can be engineered to contain a cDNA that encodes an antisense molecule that prevents an enzyme from being made. The term “antisense molecule” as used herein encompasses any nucleic acid molecule that contains sequences that correspond to the coding strand of an endogenous polypeptide. An antisense molecule also can have flanking sequences (e.g., regulatory sequences). Thus antisense molecules can be ribozymes or antisense oligonucleotides. A ribozyme can have any general structure including, without limitation, hairpin, hammerhead, or axhead structures, provided the molecule cleaves RNA.

Yeasts having a reduced enzymatic activity can be identified using many methods. For example, yeasts having reduced acetolactate decarboxylase, diacetyl reductase, acetoin reductase, 3-KAR, ALDH, PDC, or GPD activity can be easily identified using common methods, which may include, for example, measuring for the formation of the by-products produced by such enzymes via liquid chromatography.

### Overexpression of Heterologous Genes

Methods for overexpressing a polypeptide from a native or heterologous nucleic acid molecule are well known. Such methods include, without limitation, constructing a nucleic acid sequence such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous genes can be under the control of an inducible promoter or a constitutive promoter. Moreover, methods for expressing a polypeptide from an exogenous nucleic acid molecule in yeast are well known. For example, nucleic acid constructs that are used for the expression of exogenous polypeptides within Kluyveromyces and Saccharomyces are well known (see, e.g., U.S. Pat. Nos. 4,859,596 and 4,943,529, for Kluyveromyces and, e.g., Gellissen et al., Gene 190(1):87-97 (1997) for Saccharomyces). Yeast plasmids have a selectable marker and an origin of replication. In addition certain plasmids may also contain a centromeric sequence. These centromeric plasmids are generally a single or low copy plasmid. Plasmids without a centromeric sequence and utilizing either a 2 micron (S. cerevisiae) or 1.6 micron (K. lactis) replication origin are high copy plasmids. The selectable marker can be either prototrophic, such as HIS3, TRP1, LEU2, URA3 or ADE2, or antibiotic resistance, such as, bar, ble, hph, or kan.

In another embodiment, heterologous control elements can be used to activate or repress expression of endogenous genes. Additionally, when expression is to be repressed or eliminated, the gene for the relevant enzyme, protein or RNA can be eliminated by known deletion techniques.

As described herein, any yeast within the scope of the disclosure can be identified by selection techniques specific to the particular enzyme being expressed, over-expressed or repressed. Methods of identifying the strains with the desired phenotype are well known to those skilled in the art. Such methods include, without limitation, PCR, RT-PCR, and nucleic acid hybridization techniques such as Northern and Southern analysis, altered growth capabilities on a particular substrate or in the presence of a particular substrate, a chemical compound, a selection agent and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular yeast cell contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding acetolactate synthase and detecting increased acetolactate concentrations compared to a cell without the vector indicates that the vector is both present and that the gene product is active. Methods for detecting specific enzymatic activities or the presence of particular products are well known to those skilled in the art. For example, the presence of acetolactate can be determined as described by Hugenholtz and Starrenburg, 1992, Appl. Micro. Biot. 38:17-22.

### Increase of Enzymatic Activity

Yeast microorganisms of the invention may be further engineered to have increased activity of enzymes (e.g., increased activity of enzymes involved in an isobutanol producing metabolic pathway). The term “increased” as used herein with respect to a particular enzymatic activity refers to a higher level of enzymatic activity than that measured in a comparable yeast cell of the same species. For example, overexpression of a specific enzyme can lead to an increased level of activity in the cells for that enzyme. Increased activities for enzymes involved in glycolysis or the isobutanol pathway would result in increased productivity and yield of isobutanol.

Methods to increase enzymatic activity are known to those skilled in the art. Such techniques may include increasing the expression of the enzyme by increased copy number and/or use of a strong promoter, introduction of mutations to relieve negative regulation of the enzyme, introduction of specific mutations to increase specific activity and/or decrease the K_{M} for the substrate, or by directed evolution. See, e.g., Methods in Molecular Biology (vol. 231), ed. Arnold and Georgiou, Humana Press (2003).

### Methods of Using Recombinant Microorganisms for High-Yield Fermentations

For a biocatalyst to produce a beneficial metabolite most economically, it is desirable to produce said metabolite at a high yield. Preferably, the only product produced is the desired metabolite, as extra products (i.e. by-products) lead to a reduction in the yield of the desired metabolite and an increase in capital and operating costs, particularly if the extra products have little or no value. These extra products also require additional capital and operating costs to separate these products from the desired metabolite.

In one aspect, the present invention provides a method of producing a beneficial metabolite derived from a recombinant microorganism comprising a biosynthetic pathway which uses acetolactate as an intermediate in a culture medium containing a feedstock providing the carbon source until a recoverable quantity of the beneficial metabolite is produced and optionally, recovering the metabolite. In an exemplary embodiment, said recombinant microorganism is engineered to reduce or eliminate the expression or activity of an enzyme catalyzing the conversion of acetolactate to 2,3-butanediol. In one embodiment, the recombinant microorganism is engineered to reduce or eliminate the expression or activity of an enzyme catalyzing the conversion of acetolactate to acetoin. In another embodiment, the recombinant microorganism is engineered to reduce or eliminate the expression or activity of an enzyme catalyzing the conversion of diacetyl to acetoin. In yet another embodiment, the recombinant microorganism is engineered to reduce or eliminate the expression or activity of an enzyme catalyzing the conversion of acetoin to 2,3-butanediol.

The beneficial metabolite may be derived from any biosynthetic pathway which uses acetolactate as intermediate, including, but not limited to, biosynthetic pathways for the production of isobutanol, 1-butanol, valine, leucine, pantothenic acid, isobutylene, 3-methyl-1-butanol, 4-methyl-1-pentanol, and coenzyme A. In a specific embodiment, the beneficial metabolite is isobutanol.

In a method to produce a desired metabolite (e.g., isobutanol) from a carbon source, the recombinant microorganism is cultured in an appropriate culture medium containing a carbon source. In certain embodiments, the method further includes isolating the desired metabolite (e.g., isobutanol) from the culture medium. For example, a desired metabolite (e.g., isobutanol) may be isolated from the culture medium by any method known to those skilled in the art, such as distillation, pervaporation, or liquid-liquid extraction.

In one embodiment, the recombinant microorganism may produce the beneficial metabolite from a carbon source at a yield of at least 5 percent theoretical. In another embodiment, the microorganism may produce the beneficial metabolite from a carbon source at a yield of at least about 10 percent, at least about 15 percent, about least about 20 percent, at least about 25 percent, at least about 30 percent, at least about 35 percent, at least about 40 percent, at least about 45 percent, at least about 50 percent, at least about 55 percent, at least about 60 percent, at least about 65 percent, at least about 70 percent, at least about 75 percent, at least about 80 percent, at least about 85 percent, at least about 90 percent, at least about 95 percent, or at least about 97.5% theoretical. In a specific embodiment, the beneficial metabolite is isobutanol.

### Distillers Dried Grains Comprising Spent Yeast Biocatalysts

In an economic fermentation process, as many of the products of the fermentation as possible, including the co-products that contain biocatalyst cell material, should have value. Insoluble material produced during fermentations using grain feedstocks, like corn, is frequently sold as protein and vitamin rich animal feed called distillers dried grains (DDG). See, e.g., commonly owned and co-pending U.S. Publication No. 2009/0215137, which is herein incorporated by reference in its entirety for all purposes. As used herein, the term “DDG” generally refers to the solids remaining after a fermentation, usually consisting of unconsumed feedstock solids, remaining nutrients, protein, fiber, and oil, as well as spent yeast biocatalysts or cell debris therefrom that are recovered by further processing from the fermentation, usually by a solids separation step such as centrifugation.

Distillers dried grains may also include soluble residual material from the fermentation, or syrup, and are then referred to as “distillers dried grains and solubles” (DDGS). Use of DDG or DDGS as animal feed is an economical use of the spent biocatalyst following an industrial scale fermentation process.

Accordingly, in one aspect, the present invention provides an animal feed product comprised of DDG derived from a fermentation process for the production of a beneficial metabolite (e.g., isobutanol), wherein said DDG comprise a spent yeast biocatalyst of the present invention. In an exemplary embodiment, said spent yeast biocatalyst has been engineered to reduce or eliminate the expression or activity of an enzyme catalyzing the conversion of acetolactate to 2,3-butanediol. In one embodiment, said spent yeast biocatalyst has been engineered to reduce or eliminate the expression or activity of an enzyme catalyzing the conversion of acetolactate to acetoin. In another embodiment, said spent yeast biocatalyst has been engineered to reduce or eliminate the expression or activity of an enzyme catalyzing the conversion of diacetyl to acetoin. In yet another embodiment, said spent yeast biocatalyst has been engineered to reduce or eliminate the expression or activity of an enzyme catalyzing the conversion of acetoin to 2,3-butanediol.

In some embodiments, the DDG or DDGS product derived from a fermentation process using a yeast biocatalyst of the present invention that has been modified to alter the expression or activity of an enzyme involved in the conversion of acetolactate to 2,3-butanediol will exhibit an altered metabolite profile as compared to the corresponding DDG or DDGS product derived from a fermentation using a yeast biocatalyst that has not been modified to alter the expression or activity of an enzyme involved in the conversion of acetolactate to 2,3-butanediol. For instance, the DDG or DDGS product derived from a fermentation process using a yeast biocatalyst that has been modified to reduce the expression or activity of an enzyme involved in the formation of acetolactate to 2,3-butanediol may exhibit a metabolite profile with a reduced amount of 2,3-butanediol. Similarly, the DDG or DDGS product derived from a fermentation process using a yeast biocatalyst that has been modified to reduce the expression or activity of an enzyme involved in the formation of acetolactate to acetoin may exhibit a metabolite profile with a reduced amount of acetoin. In contrast, the DDG or DDGS product derived from a fermentation process using a yeast biocatalyst that has been modified to increase the expression or activity of an enzyme involved in the formation of acetolactate to 2,3-butanediol may exhibit a metabolite profile with an increased amount of 2,3-butanediol. Likewise, the DDG or DDGS product derived from a fermentation process using a yeast biocatalyst that has been modified to increase the expression or activity of an enzyme involved in the formation of acetolactate to acetoin may exhibit a metabolite profile with an increased amount of acetoin. The skilled artisan—equipped with the disclosures of the instant application—would thus how to produce DDG or DDGS products with increased or decreased amounts of diacetyl, acetoin, and/or 2,3-butanediol.

In certain additional embodiments, the DDG comprising a spent yeast biocatalyst of the present invention comprise at least one additional product selected from the group consisting of unconsumed feedstock solids, nutrients, proteins, fibers, and oils.

In another aspect, the present invention provides a method for producing DDG derived from a fermentation process using a yeast biocatalyst (i.e., a recombinant yeast microorganism of the present invention), said method comprising: (a) cultivating said yeast biocatalyst in a fermentation medium comprising at least one carbon source; (b) harvesting insoluble material derived from the fermentation process, said insoluble material comprising said yeast biocatalyst; and (c) drying said insoluble material comprising said yeast biocatalyst to produce the DDG.

In certain additional embodiments, the method further comprises step (d) of adding soluble residual material from the fermentation process to said DDG to produce DDGS. In some embodiments, said DDGS comprise at least one additional product selected from the group consisting of unconsumed feedstock solids, nutrients, proteins, fibers, and oils.

This invention is further illustrated by the following examples that should not be construed as limiting. The contents of all references, patents, and published patent applications cited throughout this application, as well as the Figures and the Sequence Listing, are incorporated herein by reference for all purposes.

### EXAMPLES

### Example 1

### Involvement of Bdh1p and Bdh2p on 2,3-Butanediol Accumulation in Yeast

The following example illustrates that deletion, disruption, or mutation of the BDH1 and BDH2 genes from the yeast genome decreases accumulation of 2,3-butanediol.

Strains, plasmids, and primers used in Example 1 are listed in Tables 3, 4, and 5, respectively.

TABLE 3
Genotype of Strains Disclosed in Example 1.

| Strain No. | Genotype |
|---|---|
| GEVO6014 | MATa ura3Δ |
| | ald6::P_{PGK1}:Bs_alsS1_coSc:T_{CYC1}:P_{PGK1}:KI_URA3:T_{CYC1}:P_{CCW12}:Ec_ilvC_coSc^{P2D1-A1-his6} |
| | gpd2::P_{PDC1(-628)}:LI_ilvD_coSc4:P_{TDH3}:Sc_AFT1:T_{CYC1}:loxP:P_{CCW12}:Ec_ilvC_coSc^{P2D1-A1-his6} |
| | tma29::loxP gpd1::P_{ADH1}:Bs_alsS1_coSc:T_{CYC1}:P_{PDC1(-750)}:LI_kivD_coSc5:T_{GPD1} |
| | pdc1::P_{CUP1}:Bs_alsS1_coSc:T_{CYC1}:P_{PGK1}:LI_kivD2_coEc:T_{KI}_{_{—}}_{URA3} |
| | pdc6::P_{TEF1}:LI_ilvD:P_{TDH3}:Ec_ilvC_coSC^{P2D1-A1}:P_{ENO2}:LI_adhA |
| | pdc5::T_{KI}_{_{—}}_{LAC4} {evolved for C2i, glucose derepression and ~0.1 h⁻¹ |
| | growth rate in YNB50D medium} Carries gDNA from BUD4629 (pGV2964) G418R |
| GEVO6944 | MATa ura3Δ bdh2/1Δ::PSc_TEF1:ble:TSc_CYC1 |
| | ald6::PPGK1:Bs_alsS1_coSc:TCYC1:PPGK1:KI_URA3:TCYC1:PCCW12:Ec_ilvC_coScP2D1-A1-his6 |
| | gpd2::PPDC1(−628):LI_ilvD_coSc4:PTDH3:Sc_AFT1:TCYC1:loxP:PCCW12:Ec_ilvC_coScP2D1-A1-his6 |
| | tma29::loxP gpd1::PADH1:Bs_alsS1_coSc:TCYC1:PPDC1(−750):LI_kivD_coSc5:TGPD1 |
| | pdc1::PCUP1:Bs_alsS1_coSc:TCYC1:PPGK1:LI_kivD2_coEc:TKI_URA3 |
| | pdc6::PTEF1:LI_ilvD:PTDH3:Ec_ilvC_coSCP2D1-A1:PENO2:LI_adhA |
| | pdc5::TKI_LAC4 {evolved for C2i, glucose derepression and ~0.1 h−1 |
| | growth rate in YNB50D medium} Carries gDNA from BUD4629 (pGV2964) G418R |

TABLE 4
Plasmid Disclosed in Example 1.

| Plasmid | Plasmid Genotype |
|---|---|
| pGV2787 | pUC-ori, bla, loxP:P_{TEF1}:ble:T_{CYC1}:loxP, |

TABLE 5
Primers Disclosed in Example 1.

| Primer | Description | Sequence |
|---|---|---|
| oGV770 | Phleomycin, | GACGCGTGTACGCATGTAAC (SEQ ID NO: 153) |
| | Forward | |
| oGV821 | Phleomycin, | CGGGTAATTAACGACACCCTAGAGG (SEQ ID NO: 154) |
| | Reverse | |
| oGV3120 | 5′ junction | GCATACAGGCCGCACAAGAG (SEQ ID NO: 155) |
| | of integra- | |
| | tions at | |
| | BDH2 | |
| oGV3121 | 3′ junction | CTCATTCTTGGCTGCTGTTC (SEQ ID NO: 156) |
| | of integra- | |
| | tions at | |
| | BDH1 | |
| oGV3703 | BDH2 Int R | GTCCGTACCGCAAATACCAC (SEQ ID NO: 157) |
| oGV3704 | BDH1 Int F | AGAAGTTGTTCGTGCCATCC (SEQ ID NO: 158) |
| oGV3693 | 3′ of 5′ ble | AACTCCGCGAGGTCGTCCAG (SEQ ID NO: 159) |
| | fragment | |
| oGV3694 | 5′ of 3′ ble | AACCTGCCATCACGAGATTT (SEQ ID NO: 160) |
| | fragment | |
| oGV3743 | BDH2 loxP F | GCAATAAGAATAACAATAAATTCATTGAACATATTTCAGAATAACTTCGTATAATGTATG |
| | | (SEQ ID NO: 161) |
| oGV3744 | BDH1 loxP R | TACAAATGAGCCGCGAGGGGCCCCAAATATTATTTTGTCAATAACTTCGTATAGCATACA |
| | | TTAT (SEQ ID NO: 162) |

Preparation of PCR-Based Transformation Fragments:

Using the FailSafe™ PCR system (EPICENTRE® Biotechnologies, Madison, Wis.; Catalog #FS99250), GEVO06014 transformants were screened for deletion of the BDH2/1 region and insertion of phleomycin gene using the following primers: oGV0770, oGV0821, oGV3120, oGV3121, oGV3703, and oGV3704. (Table 5). Each screening and verification PCR reaction mix contained 10 μL 2×FailSafe™ Master Mix E, 6.7 μL water, 1.5 μL of each primer, 0.3 μL of FailSafe™ PCR Enzyme. The PCR reactions were incubated in a thermocycler using the following touchdown PCR conditions: 1 cycle of 94° C.×2 min, 10 cycles of 94° C.×20 s, 57°-47° C.×20 s (decrease 1° C. per cycle), 72° C.×75 s, 40 cycles of 94° C.×20 s, 47° C.×20 s, 72° C.×75 s and 1 cycle of 72° C.×10 min.

Shake Flask Fermentations:

Shake flask fermentations with GEVO06014 and GEVO06944 (Δbdh1/Δbdh2) strains were performed. The cultures were incubated at 250 RPM, 30° C. for 24 hours. Samples (1.5 mL) were removed from the cultures (time=0) prior to incubation at 75 RPM, 30° C. Samples were removed after 24 h and 48 h incubation. Samples were processed after determination of the optical densities (OD₆₀₀) of the cultures by centrifugation at 18,000×g, 10 minutes. The supernatants were transferred to fresh tubes and stored at 4° C. The final time point samples (48 h) were analyzed.

Results:

Table 6 shows that disruption of BDH1 and BDH2 in GEVO06014 (to produce GEVO06944) resulted in complete elimination of 2,3-butanediol production. This observation suggests that the BDH1 and BDH2 genes are responsible for acetoin reductase activity and contribute to 2,3-butanediol accumulation in yeast.

TABLE 6
Summary of isobutanol productivity and yield of isobutanol, acetoin, and 2,3-butanediol in GEVO6014 and GEVO6944.

| | | | | Specific | | YIELD (%) | | | | | | | | | |
|---|---|---|---|---|---|---|---|---|---|---|---|---|---|---|---|
| | | | | Productivity | | | | | | | | | | | Total |
| | Relevant | | | (g/(g*L)) | | | Acetoin | | Butanediol | | | | | | (Acetolactate |
| Strain | Genotype | Buffer | pH | 0 to 24 hr | 0 to 48 hr | iBuOH | 1 | 2 | S,S | R,R | meso | DHIV | IBA | Total | byproducts) |
| GEVO6014 | BDH2/1 | MES | 6.5 | 0.05 ± | 0.08 ± | 56 ± | 1 ± | 1 ± | 0 ± | 3 ± | 4 ± | 0 ± | 4 ± | 70% | 10% |
| | | | | 0.00 | 0.00 | 1% | 0% | 0% | 0% | 0% | 0% | 0% | 0% | | |
| GEVO6944 | bdh2/1Δ | MES | 6.5 | 0.05 ± | 0.08 ± | 55 ± | 3 ± | 5 ± | 0 ± | 0 ± | 1 ± | 0 ± | 3 ± | 67% | 9% |
| | | | | 0.00 | 0.00 | 2% | 0% | 0% | 0% | 0% | 0% | 0% | 0% | | |

The foregoing detailed description has been given for clearness of understanding only and no unnecessary limitations should be understood there from as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

1.-105. (canceled)

106. A recombinant yeast microorganism comprising:
(a) an exogenous gene encoding an acetolactate synthase, and
(b) at least one genetic modification which reduces the endogenous activity of one or more of the following:
(i) one or more enzymes catalyzing the conversion of acetolactate to acetoin;
(ii) one or more enzymes catalyzing the conversion of diacetyl to acetoin; and
(iii) one or more enzymes catalyzing the conversion of acetoin to 2,3-butanediol.

107. The recombinant yeast microorganism of claim 106, wherein said enzyme catalyzing the conversion of acetolactate to acetoin is an acetolactate decarboxylase.

108. The recombinant yeast microorganism of claim 106, wherein said enzyme catalyzing the conversion of diacetyl to acetoin is a diacetyl reductase.

109. The recombinant yeast microorganism of claim 108, wherein said diacetyl reductase is Oye2p.

110. The recombinant yeast microorganism of claim 108, wherein said diacetyl reductase is Ara1p.

111. The recombinant yeast microorganism of claim 108, wherein said diacetyl reductase is selected from the group consisting of: Bdh1p, Bdh2p, Erg19p, Gcy1p, Gre3p, Oye3p, Trr1p, Ypr1p, Zwf1p, and YPL088W.

112. The recombinant yeast microorganism of claim 106, wherein said enzyme catalyzing the conversion of acetoin to 2,3-butanediol is an acetoin reductase.

113. The recombinant yeast microorganism of claim 112, wherein said acetoin reductase is Bdh1p.

114. The recombinant yeast microorganism of claim 112, wherein said acetoin reductase is Bdh2p.

115. The recombinant yeast microorganism of claim 112, wherein said acetoin reductase is Ara1p.

116. The recombinant yeast microorganism of claim 112, wherein said acetoin reductase is selected from the group consisting of: Erg19p, Gcy1p, Gre3p, Oye2p, Oye3p, Trr1p, Ypr1p, Zwf1p, and YPL088W.

117. The recombinant yeast microorganism of claim 106, wherein said recombinant yeast microorganism is engineered to reduce the endogenous activity of a 3-keto acid reductase enzyme catalyzing the conversion of acetolactate to 2,3-dihydroxy-2-methylbutanoic acid (DH2 MB), and wherein said 3-keto acid reductase enzyme is YMR226c.

118. The recombinant yeast microorganism of claim 106, wherein said recombinant yeast microorganism is engineered to reduce the endogenous activity of an aldehyde dehydrogenase enzyme catalyzing the conversion of an aldehyde to an acid by-product, and wherein said aldehyde dehydrogenase enzyme is Ald6p.

119. The recombinant yeast microorganism of claim 106, wherein said recombinant yeast microorganism is engineered to reduce endogenous pyruvate decarboxylase activity.

120. The recombinant yeast microorganism of claim 106, wherein said recombinant yeast microorganism is engineered to reduce endogenous glycerol-3-phosphate dehydrogenase activity.

121. The recombinant yeast microorganism of claim 106, wherein said recombinant yeast microorganism further comprises exogenous genes encoding a ketol-acid reductoisomerase, a dihydroxyacid dehydratase, a 2-keto-acid decarboxylase, and an alcohol dehydrogenase.

122. The recombinant yeast microorganism of claim 121, wherein said ketol-acid reductoisomerase is an NADH-dependent ketol-acid reductoisomerase.

123. The recombinant yeast microorganism of claim 121, wherein said alcohol dehydrogenase is an NADH-dependent alcohol dehydrogenase.

124. The recombinant yeast microorganism of claim 106, wherein said recombinant yeast microorganism is Saccharomyces cerevisiae.

125. A method of producing isobutanol, comprising:
(a) providing a recombinant yeast microorganism according to claim 121;
(b) cultivating the recombinant yeast microorganism in a culture medium containing a feedstock providing the carbon source, until a recoverable quantity of isobutanol is produced.


SEQUENCE LISTING (162 sequences)

SEQ ID NO: 1 - DNA, 1203 bp, Saccharomyces cerevisiae
atgccatttgttaaggactttaagccacaagctttgggtgacaccaacttattcaaaccaatcaaaattggtaacaatgaacttctacaccgtgctgtcattcctccattgactagaatgagagcccaacatccaggtaatattccaaacagagactgggccgttgaatactacgctcaacgtgctcaaagaccaggaaccttgattatcactgaaggtacctttccctctccacaatctgggggttacgacaatgctccaggtatctggtccgaagaacaaattaaagaatggaccaagattttcaaggctattcatgagaataaatcgttcgcatgggtccaattatgggttctaggttgggctgctttcccagacacccttgctagggatggtttgcgttacgactccgcttctgacaacgtgtatatgaatgcagaacaagaagaaaaggctaagaaggctaacaacccacaacacagtataacaaaggatgaaattaagcaatacgtcaaagaatacgtccaagctgccaaaaactccattgctgctggtgccgatggtgttgaaatccacagcgctaacggttacttgttgaaccagttcttggacccacactccaataacagaaccgatgagtatggtggatccatcgaaaacagagcccgtttcaccttggaagtggttgatgcagttgtcgatgctattggccctgaaaaagtcggtttgagattgtctccatatggtgtcttcaacagtatgtctggtggtgctgaaaccggtattgttgctcaatatgcttatgtcttaggtgaactagaaagaagagctaaagctggcaagcgtttggctttcgtccatctagttgaacctcgtgtcaccaacccatttttaactgaaggtgaaggtgaatacaatggaggtagcaacaaatttgcttattctatctggaagggcccaattattagagctggtaactttgctctgcacccagaagttgtcagagaagaggtgaaggatcctagaacattgatcggttacggtagattttttatctctaatccagatttggttgatcgtttggaaaaagggttaccattaaacaaatatgacagagacactttctacaaaatgtcagctgagggatacattgactaccctacgtacgaagaagctctaaaactcggttgggacaaaaattaa

SEQ ID NO: 2 - protein, 400 aa, Saccharomyces cerevisiae
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 3 - DNA, 1035 bp, Saccharomyces cerevisiae
atgtcttcttcagtagcctcaaccgaaaacatagtcgaaaatatgttgcatccaaagactacagaaatatacttttcactcaacaatggtgttcgtatcccagcactgggtttggggacagcaaatcctcacgaaaagttagctgaaacaaaacaagccgtaaaagctgcaatcaaagctggatacaggcacattgatactgcttgggcctacgagacagagccattcgtaggtgaagccatcaaggagttattagaagatggatctatcaaaagggaggatcttttcataaccacaaaagtgtggccggttctatgggacgaagtggacagatcattgaatgaatctttgaaagctttaggcttggaatacgtcgacttgctcttgcaacattggccgctatgttttgaaaagattaaggaccctaaggggatcagcggactggtgaagactccggttgatgattctggaaaaacaatgtatgctgccgacggtgactatttagaaacttacaagcaattggaaaaaatttaccttgatcctaacgatcatcgtgtgagagccattggtgtctcaaatttttccattgagtatttggaacgtctcattaaggaatgcagagttaagccaacggtgaaccaagtggaaactcaccctcacttaccacaaatggaactaagaaagttctgctttatgcacgacattctgttaacagcatactcaccattaggttcccatggcgcaccaaacttgaaaatcccactagtgaaaaagcttgccgaaaagtacaatgtcacaggaaatgacttgctaatttcttaccatattagacaaggcactatcgtaattccgagatccttgaatccagttaggatttcctcgagtattgaattcgcatctttgacaaaggatgaattacaagagttgaacgacttcggtgaaaaatacccagtgagattcatcgatgagccatttgcagccatccttccagagtttactggtaacggaccaaacttggacaatttaaagtattaa

SEQ ID NO: 4 - protein, 344 aa, Saccharomyces cerevisiae
MSSSVASTENIVENMLHPKTTEIYFSLNNGVRIPALGLGTANPHEKLAETKQAVKAAIKAGYRHIDTAWAYETEPFVGEAIKELLEDGSIKREDLFITTKVWPVLWDEVDRSLNESLKALGLEYVDLLLQHWPLCFEKIKDPKGISGLVKTPVDDSGKTMYAADGDYLETYKQLEKIYLDPNDHRVRAIGVSNFSIEYLERLIKECRVKPTVNQVETHPHLPQMELRKFCFMHDILLTAYSPLGSHGAPNLKIPLVKKLAEKYNVTGNDLLISYHIRQGTIVIPRSLNPVRISSSIEFASLTKDELQELNDFGEKYPVRFIDEPFAAILPEFTGNGPNLDNLKY

SEQ ID NO: 5 - DNA, 1149 bp, Saccharomyces cerevisiae
atgagagctttggcatatttcaagaagggtgatattcacttcactaatgatatccctaggccagaaatccaaaccgacgatgaggttattatcgacgtctcttggtgtgggatttgtggctcggatcttcacgagtacttggatggtccaatcttcatgcctaaagatggagagtgccataaattatccaacgctgctttacctctggcaatgggccatgagatgtcaggaattgtttccaaggttggtcctaaagtgacaaaggtgaaggttggcgaccacgtggtcgttgatgctgccagcagttgtgcggacctgcattgctggccacactccaaattttacaattccaaaccatgtgatgcttgtcagaggggcagtgaaaatctatgtacccacgccggttttgtaggactaggtgtgatcagtggtggctttgctgaacaagtcgtagtctctcaacatcacattatcccggttccaaaggaaattcctctagatgtggctgctttagttgagcctctttctgtcacctggcatgctgttaagatttctggtttcaaaaaaggcagttcagccttggttcttggtgcaggtcccattgggttgtgtaccattttggtacttaagggaatgggggctagtaaaattgtagtgtctgaaattgcagagagaagaatagaaatggccaagaaactgggcgttgaggtgttcaatccctccaagcacggtcataaatctatagagatactacgtggtttgaccaagagccatgatgggtttgattacagttatgattgttctggtattcaagttactttcgaaacctctttgaaggcattaacattcaaggggacagccaccaacattgcagtttggggtccaaaacctgtcccattccaaccaatggatgtgactctccaagagaaagttatgactggttcgatcggctatgttgtcgaagccttcgaagaagttgttcgtgccatccacaacggagacatcgccatggaagattgtaagcaactaatcactggtaagcaaaggattgaggacggttgggaaaagggattccaagagttgatggatcacaaggaatccaacgttaagattctattgacgcctaacaatcacggtgaaatgaagtaa

SEQ ID NO: 6 - protein, 382 aa, Saccharomyces cerevisiae
MRALAYFKKGDIHFTNDIPRPEIQTDDEVIIDVSWCGICGSDLHEYLDGPIFMPKDGECHKLSNAALPLAMGHEMSGIVSKVGPKVTKVKVGDHVVVDAASSCADLHCWPHSKFYNSKPCDACQRGSENLCTHAGFVGLGVISGGFAEQVVVSQHHIIPVPKEIPLDVAALVEPLSVTWHAVKISGFKKGSSALVLGAGPIGLCTILVLKGMGASKIVVSEIAERRIEMAKKLGVEVFNPSKHGHKSIEILRGLTKSHDGFDYSYDCSGIQVTFETSLKALTFKGTATNIAVWGPKPVPFQPMDVTLQEKVMTGSIGYVVEAFEEVVRAIHNGDIAMEDCKQLITGKQRIEDGWEKGFQELMDHKESNVKILLTPNNHGEMK

SEQ ID NO: 7 - DNA, 1254 bp, Saccharomyces cerevisiae
atgagagccttagcgtatttcggtaaaggtaacatcagattcaccaaccatttaaaggagccacatattgtggcgcccgatgagcttgtgattgatatcgaatggtgtggtatttgcggtacggacctgcatgagtacacagatggtcctatctttttcccagaagatggacacacacatgagattagtcataacccattgccacaggcgatgggccacgaaatggctggtaccgttttggaggtgggccctggtgtgaaaaacttgaaagtgggagacaaggtagttgtcgagcccacaggtacatgcagagaccggtatcgttggcccctgtcgccaaacgttgacaaggaatggtgcgctgcttgcaaaaagggctactataacatttgttcatatttggggctttgtggtgcgggtgtgcagagcggtggatttgcagaacgtgttgtgatgaacgaatctcactgctacaaagtaccggacttcgtgcccttagacgttgcagctttgattcaaccgttggctgtgtgctggcatgcaattagagtctgcgagttcaaagcaggctctacggctttgatcattggtgctggccccatcggactgggcacgatactggcgttgaacgctgcaggttgcaaggacatcgtcgtttcagagcctgccaaggtaagaagagaactggctgaaaaaatgggtgccagggtttacgacccaactgcgcacgctgccaaggagagcattgattatctgaggtcgattgctgatggtggagacggcttcgattacacatttgattgctccgggttggaagtcacattgaatgctgctattcagtgtctcactttcagaggcaccgcagtgaacttggccatgtggggccatcacaagatacagttttctccgatggacatcacattgcatgaaagaaagtacacagggtccatgtgctacacacaccacgattttgaggcagtaatagaagctttggaagaaggcaggattgacattgatagagcaagacatatgataacgggcagagtcaacattgaggacggccttgatggcgccatcatgaagctgataaacgagaaggagtctacaatcaagattattctgactccaaacaatcacggagagttgaacagggaagccgataatgagaagaaagaaatttccgagctgagcagtcggaaagatcaagaaagactacgagaatcaataaacgaggctaaactgcgtcacacatga

SEQ ID NO: 8 - protein, 417 aa, Saccharomyces cerevisiae
MRALAYFGKGNIRFTNHLKEPHIVAPDELVIDIEWCGICGTDLHEYTDGPIFFPEDGHTHEISHNPLPQAMGHEMAGTVLEVGPGVKNLKVGDKVVVEPTGTCRDRYRWPLSPNVDKEWCAACKKGYYNICSYLGLCGAGVQSGGFAERVVMNESHCYKVPDFVPLDVAALIQPLAVCWHAIRVCEFKAGSTALIIGAGPIGLGTILALNAAGCKDIVVSEPAKVRRELAEKMGARVYDPTAHAAKESIDYLRSIADGGDGFDYTFDCSGLEVTLNAAIQCLTFRGTAVNLAMWGHHKIQFSPMDITLHERKYTGSMCYTHHDFEAVIEALEEGRIDIDRARHMITGRVNIEDGLDGAIMKLINEKESTIKIILTPNNHGELNREADNEKKEISELSSRKDQERLRESINEAKLRHT

SEQ ID NO: 9 - DNA, 1191 bp, Saccharomyces cerevisiae
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 10 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 11 - DNA, 939 bp, Saccharomyces cerevisiae
atgcctgctactttacatgattctacgaaaatcctttctctaaatactggagcccaaatccctcaaataggtttaggtacgtggcagtcgaaagagaacgatgcttataaggctgttttaaccgctttgaaagatggctaccgacacattgatactgctgctatttaccgtaatgaagaccaagtcggtcaagccatcaaggattcaggtgttcctcgggaagaaatctttgttactacaaagttatggtgtacacaacaccacgaacctgaagtagcgctggatcaatcactaaagaggttaggattggactacgtagacttatatttgatgcattggcctgccagattagatccagcctacatcaaaaatgaagacatcttgagtgtgccaacaaagaaggatggttctcgtgcagtggatatcaccaattggaatttcatcaaaacctgggaattaatgcaggaactaccaaagactggtaaaactaaggccgttggagtctccaacttttctataaataacctgaaagatctattagcatctcaaggtaataagcttacgccagctgctaaccaagtcgaaatacatccattactacctcaagacgaattgattaatttttgtaaaagtaaaggcattgtggttgaagcttattctccgttaggtagtaccgatgctccactattgaaggaaccggttatccttgaaattgcgaagaaaaataacgttcaacccggacacgttgttattagctggcacgtccaaagaggttatgttgtcttgccaaaatctgtgaatcccgatcgaatcaaaacgaacaggaaaatatttactttgtctactgaggactttgaagctatcaataacatatcgaaggaaaagggcgaaaaaagggttgtacatccaaattggtctcctttcgaagtattcaagtaa

SEQ ID NO: 12 - protein, 312 aa, Saccharomyces cerevisiae
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFK

SEQ ID NO: 13 - DNA, 983 bp, Saccharomyces cerevisiae
atgtcttcactggttactcttaataacggtctgaaaatgcccctagtcggcttagggtgctggaaaattgacaaaaaagtctgtgcgaatcaaatttatgaagctatcaaattaggctaccgtttattcgatggtgcttgcgactacggcaacgaaaaggaagttggtgaaggtatcaggaaagccatctccgaaggtcttgtttctagaaaggatatatttgttgtttcaaagttatggaacaattttcaccatcctgatcatgtaaaattagctttaaagaagaccttaagcgatatgggacttgattatttagacctgtattatattcacttcccaatcgccttcaaatatgttccatttgaagagaaataccctccaggattctatacgggcgcagatgacgagaagaaaggtcacatcaccgaagcacatgtaccaatcatagatacgtaccgggctctggaagaatgtgttgatgaaggcttgattaagtctattggtgtttccaactttcagggaagcttgattcaagatttattacgtggttgtagaatcaagcccgtggctttgcaaattgaacaccatccttatttgactcaagaacacctagttgagtttgtaaattacacgatatccaagtagttgcttactcctccttcggtcctcaatcattcattgagatggacttacagttggcaaaaaccacgccaactctgttcgagaatgatgtaatcaagaaggtctcacaaaaccatccaggcagtaccacttcccaagtattgcttagatgggcaactcagagaggcattgccgtcattccaaaatcttccaagaaggaaaggttacttggcaacctagaaatcgaaaaaaagttcactttaacggagcaagaattgaaggatatttctgcactaaatgccaacatcagatttaatgatccatggacctggttggatggtaaattccccacttttgcctga

SEQ ID NO: 14 - protein, 327 aa, Saccharomyces cerevisiae
MSSLVTLNNGLKMPLVGLGCWKIDKKVCANQIYEAIKLGYRLFDGACDYGNEKEVGEGIRKAISEGLVSRKDIFVVSKLWNNFHHPDHVKLALKKTLSDMGLDYLDLYYIHFPIAFKYVPFEEKYPPGFYTGADDEKKGHITEAHVPIIDTYRALEECVDEGLIKSIGVSNFQGSLIQDLLRGCRIKPVALQIEHHPYLTQEHLVEFCKLHDIQVVAYSSFGPQSFIEMDLQLAKTTPTLFENDVIKKVSQNHPGSTTSQVLLRWATQRGIAVIPKSSKKERLLGNLEIEKKFTLTEQELKDISALNANIRFNDPWTWLDGKFPTFA

SEQ ID NO: 15 - DNA, 1203 bp, Saccharomyces cerevisiae
atgccatttgtaaaaggttttgagccgatctccctaagagacacaaacctttttgaaccaattaagattggtaacactcagcttgcacatcgtgcggttatgcccccattgaccagaatgagggccactcaccccggaaatattccaaataaggagtgggctgctgtgtattatggtcagcgtgctcaaagacctggtaccatgatcatcacggaaggtacgtttatttcccctcaagccggcggctatgacaacgcccctgggatttggtctgatgagcaggtcgctgagtggaagaatatctttttagccatccatgattgtcagtcgttcgcgtgggtacaactttggtctttaggctgggcatccttcccagacgtattggcaagagacgggttacgctatgactgtgcatctgacagagtgtatatgaatgctacgttacaagaaaaggccaaagatgcgaataatctcgaacatagtttgactaaagacgacattaaacagtatatcaaggattacatccatgcggctaagaattctatcgcggctggcgccgatggtgtagaaattcatagcgccaatgggtacttgttgaatcagttcttggatccacattctaataagaggaccgacgaatacggcggaacgatcgaaaacagggcccgctttacactggaggttgtcgatgctcttatcgaaactatcggtcctgaacgggtgggtttgaggttgtcgccgtacggcacttttaacagtatgtctgggggtgctgaaccaggtattatcgctcaatattcgtatgttttgggtgaattagagaagagggcaaaggctggtaagcgtttggcctttgtgcacctcgttgaaccacgtgtcacggacccatcgttggtggagggcgaaggagaatattccgagggtactaacgattttgcctactctatatggaagggtccaatcatcagagctggtaattacgctcttcatccagaagtggttagagaacaagtaaaggatcccagaaccttgataggctatggtagattcttcatctctaacccagatttagtctaccgtttagaagagggcctgccattgaacaagtatgacagaagtaccttctacaccatgtccgcggaaggttataccgactacccaacatatgaagaggcagtagatttaggttggaacaagaactga

SEQ ID NO: 16 - protein, 400 aa, Saccharomyces cerevisiae
MPFVKGFEPISLRDTNLFEPIKIGNTQLAHRAVMPPLTRMRATHPGNIPNKEWAAVYYGQRAQRPGTMIITEGTFISPQAGGYDNAPGIWSDEQVAEWKNIFLAIHDCQSFAWVQLWSLGWASFPDVLARDGLRYDCASDRVYMNATLQEKAKDANNLEHSLTKDDIKQYIKDYIHAAKNSIAAGADGVEIHSANGYLLNQFLDPHSNKRTDEYGGTIENRARFTLEVVDALIETIGPERVGLRLSPYGTFNSMSGGAEPGIIAQYSYVLGELEKRAKAGKRLAFVHLVEPRVTDPSLVEGEGEYSEGTNDFAYSIWKGPIIRAGNYALHPEVVREQVKDPRTLIGYGRFFISNPDLVYRLEEGLPLNKYDRSTFYTMSAEGYTDYPTYEEAVDLGWNKN

SEQ ID NO: 17 - DNA, 960 bp, Saccharomyces cerevisiae
atggttcacaacaaagttactatcattggttcaggtccagctgcacacaccgccgccatctatttggccagggcagaaatcaagccaatcctatatgaaggtatgatggcgaacggtattgctgccggtggccagctaaccaccactacagaaatcgaaaacttcccaggtttcccagatggtctaacaggtagcgaactgatggacagaatgagagaacaatccacgaagtttggcactgaaattatcacggaaacagtttccaaagttgatctgtcttccaaaccattcaagctatggaccgaatttaacgaagacgcagaacctgtgacgactgacgctataatcttggccacaggcgcttctgctaagagaatgcatttgccgggcgaggaaacctactggcaaaaaggtatttctgcctgtgccgtgtgtgatggtgccgtccccattttcagaaacaagccattggccgtcattggtggtggtgactctgcatgtgaagaagctcagttcttgaccaagtacggctcaaaagtgtttatgcttgtcagaaaagaccatttgcgtgcttctaccattatgcaaaagcgtgctgagaagaacgaaaaaattgaaatcctttacaacactgttgcgctagaagctaagggggatggtaaattattgaatgccttgaggattaagaacactaaaaagaatgaagaaaccgatttgccagtcagcggtttattttatgcaattggccacactccagcaacaaagattgtcgctggccaagtcgacactgatgaagcgggctacattaaaactgtcccaggcagctcattaacctccgttccaggatttttcgctgctggtgatgttcaggattctaaatacagacaagctattacttctgctggctctggttgtatggccgctttggatgctgagaaatacttaacttccctagaatag

SEQ ID NO: 18 - protein, 319 aa, Saccharomyces cerevisiae
MVHNKVTIIGSGPAAHTAAIYLARAEIKPILYEGMMANGIAAGGQLTTTTEIENFPGFPDGLTGSELMDRMREQSTKFGTEIITETVSKVDLSSKPFKLWTEFNEDAEPVTTDAIILATGASAKRMHLPGEETYWQKGISACAVCDGAVPIFRNKPLAVIGGGDSACEEAQFLTKYGSKVFMLVRKDHLRASTIMQKRAEKNEKIEILYNTVALEAKGDGKLLNALRIKNTKKNEETDLPVSGLFYAIGHTPATKIVAGQVDTDEAGYIKTVPGSSLTSVPGFFAAGDVQDSKYRQAITSAGSGCMAALDAEKYLTSLE

SEQ ID NO: 19 - DNA, 939 bp, Saccharomyces cerevisiae
atgcctgctacgttaaagaattcttctgctacattaaaactaaatactggtgcctccattccagtgttgggtttcggcacttggcgttccgttgacaataacggttaccattctgtaattgcagctttgaaagctggatacagacacattgatgctgcggctatctatttgaatgaagaagaagttggcagggctattaaagattccggagtccctcgtgaggaaatttttattactactaagctttggggtacggaacaacgtgatccggaagctgctctaaacaagtctttgaaaagactaggcttggattatgttgacctatatctgatgcattggccagtgcctttgaaaaccgacagagttactgatggtaacgttctgtgcattccaacattagaagatggcactgttgacatcgatactaaggaatggaattttatcaagacgtgggagttgatgcaagagttgccaaagacgggcaaaactaaagccgttggtgtctctaatttttctattaacaacattaaagaattattagaatctccaaataacaaggtggtaccagctactaatcaaattgaaattcatccattgctaccacaagacgaattgattgccttttgtaaggaaaagggtattgttgttgaagcctactcaccatttgggagtgctaatgctcctttactaaaagagcaagcaattattgatatggctaaaaagcacggcgttgagccagcacagcttattatcagttggagtattcaaagaggctacgttgttctggccaaatcggttaatcctgaaagaattgtatccaattttaagattttcactctgcctgaggatgatttcaagactattagtaacctatccaaagtgcatggtacaaagagagtcgttgatatgaagtggggatccttcccaattttccaatga

SEQ ID NO: 20 - protein, 312 aa, Saccharomyces cerevisiae
MPATLKNSSATLKLNTGASIPVLGFGTWRSVDNNGYHSVIAALKAGYRHIDAAAIYLNEEEVGRAIKDSGVPREEIFITTKLWGTEQRDPEAALNKSLKRLGLDYVDLYLMHWPVPLKTDRVTDGNVLCIPTLEDGTVDIDTKEWNFIKTWELMQELPKTGKTKAVGVSNFSINNIKELLESPNNKVVPATNQIEIHPLLPQDELIAFCKEKGIVVEAYSPFGSANAPLLKEQAIIDMAKKHGVEPAQLIISWSIQRGYVVLAKSVNPERIVSNFKIFTLPEDDFKTISNLSKVHGTKRVVDMKWGSFPIFQ

SEQ ID NO: 21 - DNA, 1518 bp, Saccharomyces cerevisiae
atgagtgaaggccccgtcaaattcgaaaaaaataccgtcatatctgtctttggtgcgtcaggtgatctggcaaagaagaagacttttcccgccttatttgggcttttcagagaaggttaccttgatccatctaccaagatcttcggttatgcccggtccaaattgtccatggaggaggacctgaagtcccgtgtcctaccccacttgaaaaaacctcacggtgaagccgatgactctaaggtcgaacagttcttcaagatggtcagctacatttcgggaaattacgacacagatgaaggcttcgacgaattaagaacgcagatcgagaaattcgagaaaagtgccaacgtcgatgtcccacaccgtctcttctatctggccttgccgccaagcgtttttttgacggtggccaagcagatcaagagtcgtgtgtacgcagagaatggcatcacccgtgtaatcgtagagaaacctttcggccacgacctggcctctgccagggagctgcaaaaaaacctggggcccctctttaaagaagaagagttgtacagaattgaccattacttgggtaaagagttggtcaagaatcttttagtcttgaggttcggtaaccagtttttgaatgcctcgtggaatagagacaacattcaaagcgttcagatttcgtttaaagagaggttcggcaccgaaggccgtggcggctatttcgactctataggcataatcagagacgtgatgcagaaccatctgttacaaatcatgactctcttgactatggaaagaccggtgtcttttgacccggaatctattcgtgacgaaaaggttaaggttctaaaggccgtggcccccatcgacacggacgacgtcctcttgggccagtacggtaaatctgaggacgggtctaagcccgcctacgtggatgatgacactgtagacaaggactctaaatgtgtcacttttgcagcaatgactttcaacatcgaaaacgagcgttgggagggcgtccccatcatgatgcgtgccggtaaggctttgaatgagtccaaggtggagatcagactgcagtacaaagcggtcgcatcgggtgtcttcaaagacattccaaataacgaactggtcatcagagtgcagcccgatgccgctgtgtacctaaagtttaatgctaagacccctggtctgtcaaatgctacccaagtcacagatctgaatctaacttacgcaagcaggtaccaagacttttggattccagaggcttacgaggtgttgataagagacgccctactgggtgaccattccaactttgtcagagatgacgaattggatatcagttggggcatattcaccccattactgaagcacatagagcgtccggacggtccaacaccggaaatttacccctacggatcaagaggtccaaagggattgaaggaatatatgcaaaaacacaagtatgttatgcccgaaaagcacccttacgcttggcccgtgactaagccagaagatacgaaggataattag

SEQ ID NO: 22 - protein, 505 aa, Saccharomyces cerevisiae
MSEGPVKFEKNTVISVFGASGDLAKKKTFPALFGLFREGYLDPSTKIFGYARSKLSMEEDLKSRVLPHLKKPHGEADDSKVEQFFKMVSYISGNYDTDEGFDELRTQIEKFEKSANVDVPHRLFYLALPPSVFLTVAKQIKSRVYAENGITRVIVEKPFGHDLASARELQKNLGPLFKEEELYRIDHYLGKELVKNLLVLRFGNQFLNASWNRDNIQSVQISFKERFGTEGRGGYFDSIGIIRDVMQNHLLQIMTLLTMERPVSFDPESIRDEKVKVLKAVAPIDTDDVLLGQYGKSEDGSKPAYVDDDTVDKDSKCVTFAAMTFNIENERWEGVPIMMRAGKALNESKVEIRLQYKAVASGVFKDIPNNELVIRVQPDAAVYLKFNAKTPGLSNATQVTDLNLTYASRYQDFWIPEAYEVLIRDALLGDHSNFVRDDELDISWGIFTPLLKHIERPDGPTPEIYPYGSRGPKGLKEYMQKHKYVMPEKHPYAWPVTKPEDTKDN

SEQ ID NO: 23 - DNA, 1029 bp, Saccharomyces cerevisiae
atggttttagttaagcaggtaagactcggtaactcaggtcttaagatatcaccgatagtgataggatgtatgtcatacgggtccaagaaatgggcggactgggtcatagaggacaagacccaaattttcaagattatgaagcattgttacgataaaggtcttcgtacttttgacacagcagatttttattctaatggtttgagtgaaagaataattaaggagtttctggagtactacagtataaagagagaaacggtggtgattatgaccaaaatttacttcccagttgatgaaacgcttgatttgcatcataacttcactttaaatgaatttgaagaattggacttgtccaaccagcggggtttatccagaaagcatataattgctggtgtcgagaactctgtgaaaagactgggcacatatatagaccttttacaaattcacagattagatcatgaaacgccaatgaaagagatcatgaaggcattgaatgatgttgttgaagcgggccacgttagatacattggggcttcgagtatgttggcaactgaatttgcagaactgcagttcacagccgataaatatggctggtttcagttcatttcttcgcagtcttactacaatttgctctatcgtgaagatgaacgcgaattgattccttttgccaaaagacacaatattggtttacttccatggtctcctaacgcacgaggcatgttgactcgtcctctgaaccaaagcacggacaggattaagagtgatccaactttcaagtcgttacatttggataatctcgaagaagaacaaaaggaaattataaatcgtgtggaaaaggtgtcgaaggacaaaaaagtctcgatggctatgctctccattgcatgggttttgcataaaggatgtcaccctattgtgggattgaacactacagcaagagtagacgaagcgattgccgcactacaagtaactctaacagaagaagagataaagtacctcgaggagccctacaaaccccagaggcaaagatgttaa

SEQ ID NO: 24 - protein, 342 aa, Saccharomyces cerevisiae
MVLVKQVRLGNSGLKISPIVIGCMSYGSKKWADWVIEDKTQIFKIMKHCYDKGLRTFDTADFYSNGLSERIIKEFLEYYSIKRETVVIMTKIYFPVDETLDLHHNFTLNEFEELDLSNQRGLSRKHIIAGVENSVKRLGTYIDLLQIHRLDHETPMKEIMKALNDVVEAGHVRYIGASSMLATEFAELQFTADKYGWFQFISSQSYYNLLYREDERELIPFAKRHNIGLLPWSPNARGMLTRPLNQSTDRIKSDPTFKSLHLDNLEEEQKEIINRVEKVSKDKKVSMAMLSIAWVLHKGCHPIVGLNTTARVDEAIAALQVTLTEEEIKYLEEPYKPQRQRC

SEQ ID NO: 25 - protein, 321 aa, Kluyveromyces thermotolerans
MLHPKNTEVYFTLNNGVRMPAIGLGTANVVEQIPQTKQAVKAAIKSGYRHIDTAWAYQCEDRVGEALKELFEEGVVKREDIFVTTKVWPTNWDRVDESISRSLENLGLDHVDLVLQHWPLCFNRLEDPDGIDGVRRNPYHEDGSPHYNEKGDYLETFKTLEKLYLAKDPRFRAIGVSNYPVEYLERLLKECQVVPAINQVECHPHLPQMELRDFCTKHNIRLEAYSPLGATNAPLIDNELVKKIAGKYACSTQNVLIAYHMRQGVVSVPRSVNPKHIASNVQFVALSKEDIDTLNKFGVENTKRIVDEPFAAAIPGFRENK

SEQ ID NO: 26 - protein, 321 aa, Kluyveromyces waltii
MLHPKNTEIYFTLNNGVKMPALGLGTANVIEQIPQTKQAVKAAIKSGYRHIDTAWAYRCEDRIGEALKELFEEGVIKREDIFITTKVWPTNWDRADESISRSLENLGVDYVDLVLQHWPLCFNRLQDPEGIDGICRNPTHEDGSPHYNEKGDYLQTFKSLEKMYLSKDPRFRSIGVSNYPVEYLERLLKECKVVPAVNQVEMHPHLPQMELRDFCTKHNIRLEAFSPFGATGSPMVKNELVQKVAQKYSCTPNDVLIAYHLRQGVVTVPRSVNPKNIASNVQFVPLSKEDIDELNKFGVENPKRLVDEEFAVAIPGFREGK

SEQ ID NO: 27 - protein, 322 aa, Saccharomyces kluyveri
MSHPKATEIYFNLNNGTRIPAVGLGTANPKDKWPQTKEAVKAAVKAGFRQIDTAWAYGTEEYIGEALQELFAEGVVKREDLFITTKVWPNYWDQAEKSLNESLKRLQLDYVDLLLQHWPFCFVKKQDPEGSGQLVNHPQHEDGSPFYDTAGDWLETYKQIEAIYLAENDTRVRAIGVSNYPIEYLERLFKECKTVPAINQVELHPRLPQRELCDYCNKHGILMTSYSPLGSNGAPNLRIPLVQELASKYNANGNDVLVSYHIRQGTIVIPRSLNLERISKGLGFVPLTKEELDKLNQYGVEHEYRYIDEAFCVCVPGFSGRD

SEQ ID NO: 28 - protein, 338 aa, Saccharomyces castellii
MSHPRNTEIYFNLNNGGRIPAIGLGTASPKGRYPETKKAVKAAIRAGYRQIDTAWYYKTEPYIGEALKELFRDGEIKREDLFITTKVWPCYWDDPSTSINESLKSLGIDYVDMVLQHWPLCYKKTYDENGTIIGKPLDKDGKVIFAEGADWITTYQLMEKIYLDPKDTRVRAIGVSNYPIEYLERVIKECKVTPVINQVELHPHLPQLELNDFCHKNGILLTAYSPLGSGGAPNTKIPLVQEYAKKHEVAPADILTSYHVRKGNVVIPRSLNPERVASNIYFAPLTKNEMKSLDDFGVTLHIDILGQTWLVLSQDSLMDVEKGKGINNVLITSLPSIT

SEQ ID NO: 29 - protein, 330 aa, Saccharomyces bayanus
MLHPKTTEIYFTLNNGVRVPALGLGTANPHEKLAETKQAVKAAIKAGYRHIDTAWAYETEPFVGEAIKELLEEGSIKREDLFITTKVWPVLWDEVERSLSESLKAMGLDYVDLFLQHWPLCFEKVKDPKGISGLVKTPVHKSGEAMLASNGDWMETYKQLEKIYLDPNDHRVRAIGVSNFSIEYLERLVKECRVKPTVNQVETHPHLPQMELRKFCFMHDILLTAYSPLGSNGAPNLKIPLVKKLVEKYNVTGNDVLISYHIRQGTIVIPRSLNPVRISSSIEFACLTKDELQELNDFGEKYPVRFIDEPFAASLPEFTGNGPNLENLKY

SEQ ID NO: 30 - protein, 330 aa, Kluyveromyces lactis
MLHPLSTEIYFTFNNGNKVPAFGLGTAAQHERVAETKQAVKAAIKAGYRHIDTAWAYGVEEYVGEALQELFEERVVKREDLHITSKVWHTMWNDVDKSLNESLARLKVDYVDLFLQHWPLCTQKVPDPHGVDKIAKEPVDDQGNPLYDEKGDWIETYKQIEKIYLDKNDKRVRAIGVSNFPIEYLKRVLKECRTVPACNQVELHPHLPQRELCEFCSEHKILVTAYSPLGGNGAPLLELPILKGLCEKYNASPNDILTSYHLRQGTIVIPRSLNSTRIASNLEFVPLSEEDVNKLNEFGQKNKKRYINGAVSSVIPGFKEDFLKEQLQQS

SEQ ID NO: 31 - protein, 328 aa, Zygosaccharomyces rouxii
MARLLHPKGTEISFTLNNGVRMPALGLGTANPHPEIGRTKQAVKAAVKAGYRHIDTAWAYGSEPFVGEAIKELMEEGVIRREDIFITSKVWPVLWDDVERSLNESLESLGVDYIDLWLQHWPLCYVKQEDTHGINGLARNPETPNGEPYYEVDGDWLETYVQMEKIYLDPSDNRVRSIGVSNFPVQYLERTLFECAVKPAVNQVEMHPRLPQFELNKFCRAHNILLEAYSPLGSHNAPNVHLQLVQDLAEKYSMSPSGILNSYHIRQGNAVVSRSVNTMRIASNVEFAALTEEELDNLDQLGITKPKRFVNEKWNAVVPGFSGEGPAI

SEQ ID NO: 32 - protein, 424 aa, Saccharomyces castellii
MHSHSLSFLLSFPSKTLSNKQTNKTNNTKQNYLTTQHNIMRALAYFGKNDIKFTNDLPEPTISAPDEMIIDVSWCGICGTDLHEFQDGPIFFPHDGCCHEISGEGLPQAMGHEIGGTIIECGPGVTKFKVGDRVVVELHGYSKDRYRWPNSHQCGQSKYHLFNLGLVGAGVQSGGFAERLVMNERQCYKVPDHMSMDVAALIQPIAVSWHAVRVSKFKKGASVLIVGAGPIGLGTILALNGHGVSEVVVSEPAKIRRDFAEKMGAIVYDPSAKDHDESIKYLRSIAPGGDGFDYAFDCSGNPATLKAAIECLTFRGTAVNVAIWGNKPVNFLPMDLTYQEKSYTGSMCYTFHDFEAVINAFEKKLIDVEKARHMITGKVPIENGVKDAILRLITHKEQTIKIILTPNNHGELGEEPEQKKKKVI

SEQ ID NO: 33 - protein, 385 aa, Kluyveromyces lactis
MRALAYFGKQDIRYTKDLEEPVIETDDGIEIEVSWCGICGSDLHEYLDGPIFFPEDGKVHDVSGLGLPQAMGHEMSGIVSKVGPKVTNIKAGDHVVVEATGTCLDHYTWPNAAHAKDAECAACQRGFYNCCAHLGFMGLGVHSGGFAEKVVVSEKHVVKIPNTLPLDVAALVEPISVSWHAVRISKLQKGQSALVLGAGPIGLATILALQGHGASKIVVSEPAEIRRNQAAKLGVETFDPSEHKEDAVNILKKLAPGGEGFDFAYDCSGVKPTFDTGVHATTFRGMYVNIAIWGHKPIDFKPMDVTLQEKFVTGSMCYTIKDFEDVVQALGNGSIAIDKARHLITGRQKIEDGFTKGFDELMNHKEKNIKILLTPNNHGELDATN

SEQ ID NO: 34 - protein, 385 aa, Saccharomyces kluyveri
MRALAYFGQKDIRFTDELPEPVISADDDLIIDIAWCGICGTDLHEYTDGPIFFPEDGHTHDISGNGLPQAMGHEMAGVVSKIGPGVQGFEVGDHVVVEPTSTCLDRYRFPNSKHSGEEPCAACKRGLYNICSHLGLCGVGVQSGGFSERVVMNQKHVIKIPKHIPLDVAALVQPLAVSWHAIRISGLTAGKSALVVGGGPIGLGTILALQAYGASKIVVSEPAKNRRELAKLMGAEVFDPTEWEEHEAVEYLRKVAPGGDGFDYSFDCSGLPVTFRASVHALTFRGTAVNVSIWGHKPIDYFPMDVTYQEKVITGSMCYTMDDFKSVLKAIVDDIIEIERAKHMITGKVRIEDGFEHAIMKLLTHKEQTIKILLTPNNNGELDGQ

SEQ ID NO: 35 - protein, 386 aa, Saccharomyces castellii
MRAIAYYKKGDIHFASDLPTPTISTPDEILIDVAWCGICGSDLHEYLDGPIFMPQDGSTHPLSNQPLPQPMGHEFCGFVKAIGNKVTKVAVGDKVVVEAGCGCKDVHRWPQSHVYGKEQCTACRKGFDNCCTHAGFTGLGVVGGAFAEQVVVGERHVVKLPEEVPTDVGALVEPLSVAWHAVKVAGFEKGSGKNALVLGAGPIGLATILALKAHGASKIVVSELAAIRRELALKMGVEVFDPSKHGDEKQGIKKLREIPEDHDGFDFAFDCSGVETTFNTALHSLTFRGTAVNVAIWGPHPIDYYPMDITLQEKKLTGSIGYVVQDFEEVVDALHKGDISIEECKHLITGRQKLENGWEKGFMELMNHKETNIKILMTPNNHHELQ

SEQ ID NO: 36 - protein, 383 aa, Candida glabrata
MRSLAYFKTGDIHFTDKIDTPKINNDHELMIDVAWCGICGTDLHEFLEGPIFMPKDGDTHYLSGLDLPLPMGHEMSGIVKEVGKGVTRFKPGDRVVIEATTSCGDTHRWPKSKVAKGELCPACEMGLPNCCTHGSFQGLGSVGGGFAEQVVVGEKHVVKIPDKLPLDVAALVEPLSVAWHAARLAEFKEGSNALVLGSGPIGLAMILALKGMKAGKIVVSEPAKLRRELAEKLNVVVFNPTHTRAQNAIEKLKELSPKDHGFDYTFDCSGVQDTFNAAVKAACIQGHIVNVAIWGPKPVAFHPMDMTYGEKKLTGTMGYVTRDFEEVVDAIDKGYISIEECKLLITGRVKLEDGMEKGFHELIEHKETNVKILLTPNNHGELQ

SEQ ID NO: 37 - protein, 382 aa, Kluyveromyces thermotolerans
MRALAYFGQKDIRFTDSLAQPQVSHEDEVEIDVSWCGVCGSDLHEYLDGPIFFPANGKTHAMSGQGLPQAMGHEMSGIVSKVGSDVSKVKVGDHVVVGACCTCEDRARWPDVKHEREGLCIACKTGNPNCCTDLGFCGLGCQSGGFAEKIVLSERNVVKIPNSLPLDVAALVEPISVSWHAVRISKLQPGQTALVLGAGPIGLAAVLALQSHGAGTIIVSEPADTRRQKAEALGAQTFNPTEHGDNVIEALRKLAPGGEGFNFSYDCCGTSETFSAGLHTLTPKGVAVNIAVWGPKPIDFYPMDVTTSERFVTGSICYTTQDFEEVVDAMDKGKIDINKVKTMITGRERIENGFENGIMDLINNKEKNIKILLTPNNFKELA

SEQ ID NO: 38 - protein, 417 aa, Saccharomyces bayanus
MRALAYFGKGDIRFTNHLEEPRIVAPDELVLDIAWCGICGTDLHEYTDGPIFFPEDGHTNEISHNPLPQAMGHEMAGTVAKVGPGVTQFKVGDKVVVEPTGTCRDRYRWPTSPNVDKEWCAACKRGLYNICSYLGLCGAGVQSGGFAERVVINESHCYKVPEFVPLDVAALIQPLAVCWHAISVCDFKAGSTALIIGAGPIGLGTILALNAAGCSDIAVSEPAKVRRELAEKMGARVFDPTAHAAKDSIDYLRSVAPGGDGYDYTFDCSGLEVTLNASVQCLTFRGTAVNLAMWGHHKVQFSPMDITLHERKYTGSMCYTHHDFEAVIEALEERRIDVAKARHMITGRVSLEEGLDGAIMKLINEKESTIKIILTPNNNGELDREAGNEKEDVSEHSTCKDQERLRESINETKLRHT

SEQ ID NO: 39 - protein, 382 aa, Saccharomyces bayanus
MRALAYFKKGDIHFTNDIPKPEIQADDEVILDVSWCGICGSDLHEYSDGPIFMPKDGECHSLSNAGLPQAMGHEMSGIIAKVGPKVTKVKVGDHVVVQAGSSCADLHCWPHSKHYNSKPCDACKKGCENLCTHAGFIGLGVISGGFAEQVVVSEHHIIPVPKEIPLDVAALVEPLSVTWHAVRLSGFKKGSSALVLGAGPIGLCTILVLKGMGAGQIVVSEVAERRMELARRLGVEIFDPRKHGEKSVESLRALTKSHDGFDYSYDCSGIQATFDASLKALTFRGTATNIAVWGPKPVPFQPMNVTLQEKIITGSIGYVVDDFKEVVQAIHSGEITIEDCKHLITGKQKIEDGWEKGFLELMNHKESNVKVLLTPNNHGEMN

SEQ ID NO: 40 - protein, 386 aa, Kluyveromyces polysporus
MRALAYFGKGNVRFTYSLKEPHIHAEDDVIIDISWCGICGTDLKEYTDGAIFFPEDGCVNHISNNNLPQAMGHEISGIISSVGPGCKKFKIGDHVVVEPTATCIDRYRWPNAPNRDTPMCEACRNGMPNICKYLGLTGAGVEGGGFAEKMVINEHHCHKIPHHMPLDVASLIQPISVCWHAVKVSHYKQGGSVLIVGGGPIGLGTILALKGFGCEDIVVSEPALIRRELAEKMGARVFDPSNMSLDNSIKTLTEMAPGSQGFDYTFDCSGIPATLKVAIECCTFRGTAVNIAVWGHKPVEFFPMDITSQEKRYTGSMCYTSEDFEEVIEAFKSKRIDINTARHLITGKVPIEEGIEGAMLRLLRDKGHTIKILLTPNNKNELGYKL

SEQ ID NO: 41 - protein, 381 aa, Kluyveromyces lactis
MRALAYFGTRDIRFTTDLPEPVIEKPDDVLIDIVWCGICGTDLHELLDGPNFFPKDGETHEISGRGLPQAMGHEMAGVVSQVGSSVTKFQPGDHVVVEPTGTCQDRERYNTKGETCKACKRGMHNVCVHLGLCGCGVQSGGFAEKVVIGEKHCFKVPSWIPLDVAALIQPLAVCWHAVKLGAVKEGQSALILGGGPIGLGIIMALQHFGCTDIVVSEPAKIRRDLAEKMGAIVSNPMDFKTNEENIKNLCSLSPNGEGYDFTFDCSGISVTFESSIKCLTFRGTAINVAVWGDSSVKHYPMDLTSQEKKYTGSMCYTLEDFEDVVDSFTEGKTDFKKAAFMITGKVSLENGMEDAFMRLINDKEGTIKILLTPKEELITWV

SEQ ID NO: 42 - protein, 382 aa, Kluyveromyces waltii
MRALAYFGQKDIRFTNDLPEPTIQASDDVQIAISWCGVCGSDLHEYLDGPIFFPKDGEKHAMSGVGLPQAMGHEMSGVVAKIGPGVKNIKVGDHVVVETTGTCKDRYRWPDVKHASDEECAACKKDFYNCCSELGFCGLGCHSGGFAEKVVISEKHVVKVPKDIPLDVAALVEPLAVSWHAVRISQLKAGQSALILGAGPIGLATVLALQGHGAGTIVVSEPSASRRKQAEALGALTFNPFDHGDKAVDELRKIPPGSDGFDFSYDCSGLKATFNTGLYALTFRGVAVNIAIWGHKPIDFYPMDVTKQEKFVTGSICYTVQDFEEVVEAMAEGRINIEKAKHMITGHEKIEDGFEKGIMDLINNKETNIKILLTPNNFNELA

SEQ ID NO: 43 - protein, 400 aa, Saccharomyces castellii
MSIHVASVTAPVNIATLKYWGKRDKQLNLPTNSSISVTLSQDDLRALTSVAASPTFTADRLWLNGKEEATVSNERTQACLSELRKLRQELEGKDTKLPKLSQWNLHIVSENNFPTAAGLASSAAGFAALVAAIAKLYKLEQSMSDLSRIARQGSGSACRSLFGGFVAWEMGEKQDGSDSQAVQVSTLDQWPNMKAAILVVSQAKKDTSSTTGMQLTVKTSDLFKERVAHVVPKRFIEMKGAINAKDFEKFAELTMKDSNSFHATCLDSFPPIFYMNDTSKKIIKLCHLINEFYKETIVAYTFDAGPNAVLYYLEENEGKLFAFVYHLFNKVEGWEKKYSQDQLNQFSSLFEDDIVPILKNSLDEEIYKGISRIILTKVGPGPQSTNECLIEESTGLPKSM

SEQ ID NO: 44 - protein, 372 aa, Ashbya gossypii
MLNLPTNSSISVTLSQEDLRTLTSAATGPELAEDRLWLNGKPESLGNARTQQCLADLRALRRALETEEPDLPRMSEWKLHIVSENNFPTAAGLASSAAGFAALVVAVAKLYGLPQDYSEISKIARKGSGSACRSLYGGYVAWEMGAEADGSDSRAVQIADVEHWPEMRAAILVVSADRKDTPSTSGMQQTVHTSDLFKERVATVVPRRYGEMAAAIRARDFATFARLTMQDSNSFHATCLDSFPPIFYMNDTSRRIVKLCHLINEFYNETIVAYTFDAGPNAVLYYLAENEARLCGFLSAVFGANDGWETTFSTEQRATFAAQFDECVRGKLATDLDDELHRGVARLIFTKVGPGPQDTKSSLIDPETGLPR

SEQ ID NO: 45 - protein, 397 aa, Kluyveromyces thermotolerans
MSIYTASTTAPVNIATLKYWGKRDKTLNLPTNSSISVTLAQEDLRTLTSVATSESFTEDQLWLNGQPESLQGERTQHCLQDLRNLRSRIEAQDSSLPRMSQWKLHIVSENNFPTAAGLASSAAGFAALVMAIAKLYQLPDSHSEISKIARKGSGSACRSLFGGYVAWEMGSEPDGSDSKAVEVAPQSHWPEMKAAILVVSADRKDTPSTSGMQHTVATSDLFQERIRNVVPKRFEEMKQAIQDRDFTRFAELTMRDSNSFHATCLDSFPPIFYMNDTSRKIVKLCHQINAFYDETIVAYTFDAGPNAVLYYLQENEAKLMAFVHHVFQKNSGWDTKFSQNDLEKFSEVFKTRVAPEVAFEFDEELYKGVSRVILTQVGPGPQDTTECLINKSTGYPN

SEQ ID NO: 46 - protein, 397 aa, Kluyveromyces lactis
MSIYSASTTAPVNIATLKYWGKRDKVLNLPTNSSISVTLSQEDLRTLTTATTSPDFAKDQLWLNGKEESLASERTQHCLQDLRQLRRELEEKDSSLPTFSQWKLHIASENNFPTAAGLASSAAGFAALIKAIAKLYELPQSESELSKIARKGSGSACRSLFGGYVAWEMGKLEDGSDSKAVEIGSLNHWPEMKAAILVVSADKKDTPSTSGMQLTVKTSDLFQERINNVVPKRFEQMKKSILEKDFPTFAELTMKDSNSFHATCLDSYPPIFYLNDTSKKVIKLCHAINEFYNETVVAYTFDAGPNAVLYYLEQSEDKLFAFLYHLFQNVSGWESKFTKEQLSQFNAKFDEIKDDVSFYLDSELHQGVTRVILTRVGPGPQDTDLSLIDSATGLPSN

SEQ ID NO: 47 - protein, 396 aa, Candida glabrata
MTYVASTTAPVNIATLKYWGKRDAKLNLPTNSSISVTLAQEDLRTLTSAATSSEFKEDKLWLNGKEESLSSERTQNCLADLRALRRQLEEKDSSLPPMSQWKLHIVSENNFPTAAGLASSAAGFAALVMAIAKLYELPQSASDISKIARKGSGSACRSLFGGYVAWEMGEKADGSDSKAVEVAPLEHWPNMKAAVLVVSADKKDTPSTSGMQLTVNTSDLFKERITNVVPKRFEAMKKAILDKDFPTFAELTMKDSNSFHATCLDSFPPIFYINDTSKKIIKLCHLINEFYGETIVAYTYDAGPNSVLYYLEENEEKLFAFIYTLFSKVDGWQSKYNSEELSKFTSTFNNQVKGKFQFDLDDTIQENVSRVILTRVGPGPQDTKECLINEETGLPK

SEQ ID NO: 48 - protein, 396 aa, Saccharomyces bayanus
MSIHTASVTAPVNIATLKYWGKRDTKLNLPTNSSISVTLSQDDLRTLTSAAAAPEFEKDTLWLNGEPHSIDNERTQNCLHDLRQLRRELELKDASLPTLSQWKLHIVSENNFPTAAGLASSAAGFAALVSAIAKLYQLPQSTSEISRIARKGSGSACRSLFGGYVAWEMGTAQDGHDSMAVQIADSSDWPQMKACVLVVSDIKKDVSSTQGMQLTVATSELFKERIEHVVPNRFEVMRKAIIEKDFATFAKETMMDSNSFHATCLDSFPPIFYMNDTSKGIISWCHSINQYYGETIVAYTFDAGPNAVLYYLAENELKLFAFIYKLFGSVPGWDKKFTAEQLDAFNQQFDSAKFTARELDLELQKGVARVILTQVGSGPQEAKESLIDAKTGLPKK

SEQ ID NO: 49 - protein, 397 aa, Saccharomyces kluyveri
MTIHSASTTAPVNIATLKYWGKRDKILNLPTNSSISVTLSQDDLRTLTSVATSPEFTEDKLWLNGKEESLASERTQNCLQDLRTLRKQLEDSDQTLPKFSQWKLHIVSENNFPTAAGLASSAAGFAALVVAISKLFQLPQSYSEISKIARKGSGSACRSLFGGYVAWEMGDKTDGSDSKAVEVAPLDHWPEMKAAVLVVSADKKDTPSTSGMQLTVHTSDLFQERIKSVVPRRFEEMKKAILDKNFPLFADLTMKDSNSFHATCLDSFPPIFYMNDISKKIIKLCHLINDFYNEIIVAYTFDAGPNAVLYYLQENESRLFAFIYKIFQKNQGWNSKFSDKQLGEFVSDFDTKIKGKLTFEIDEELYKGVTRVILTQVGPGPQETSECLIDLSTGLPK

SEQ ID NO: 50 - protein, 301 aa, Kluyveromyces waltii
MSTWKLHIVSENNFPTAAGLASSAAGFAALVVAIAKLYQLPQSYSEISKIARKGSGSACRSLFGGYVAWEMGDLADGSDSKAVEVAPASHWPNMKAAILVVSADRKDTPSTSGMQQTVATSDLFQERIKNVVPKRFEEMKEAITNRDFSRFAELTMRDSNSFHATCLDSYPPIFYMNDTSRKIVKLCHQINAFYNETIVAYTFDAGPNAVLYYLQENEAKLMSFVYQVFQKNSGWESKFSQDQLSQFSDLFKQQVASKIAFELDDELYKGVSRVILTQVGPGPQDTTETLIDQSTGNPIKK

SEQ ID NO: 51 - protein, 397 aa, Zygosaccharomyces rouxii
MVYHIASTTAPVNIATLKYWGKRDKALNLPTNSSISVTLSQEDLRTLTSAATGPELKQDKLWLNGKEESLESERTQQCLKGLRKLRKELEDKDSNLPKFSNWGLHIVSENNFPTAAGLASSAAGFAALVVAIARLYQLPQSMSELSEIARQGSGSACRSLFGGYVAWEMGEKEDGSDSKAVEISPLEHWPQMKAAILVVNASKKDTPSTSGMQLTVKTSELFQERVKNVVPQRFTHMKEAIEHKNWPKFAELTMKDSNSFHATCLDSYPPIFYMNDTSKKIIKLCHAINEFYGKTVVAYTFDAGPNAVLYYLQENEAKLFAFIYKLFDKVPGWETKFSNQDLQEFLSVYEKDVSGKLPFELDDEVQNGVSRVILTQVGPGPLSTKESLIDENTGLPK

SEQ ID NO: 52 - protein, 396 aa, Kluyveromyces polysporus
MVYVASTTAPVNIATLKYWGKRDKDLNLPTNSSISVTLAQEDLRTLTSAATDEGFTQDKLWLNGKEESLDSARTQQCLADLRGLRQQVEAQDPQAPKMSQWKLHIVSENNFPTAAGLASSAAGFAALVVAIAKLYQLPQDYSEISKIARKGSGSACRSLFGGYVAWEMGENLDGSDSKAVEVAPLNHWPNMKAAILVVSDMKKDTPSTSGMQLTVKTSDLFQERIKNVVPQRFEEMKQAIRNKDFPTFADLTMKDSNSFHATCLDSYPPIFYMNDTSRKIIRLVHSINAFYNETIVAYTYDAGPNAVLYYLEENESKLFAFIYKLFDKVSGWETKYTEAELNDFLKEFDQSVSPKLDFELDDEIYRGVSRVILTRAGPGPQDTTECLIDENTGLPK

SEQ ID NO: 53 - protein, 312 aa, Saccharomyces kluyveri
MSIQLKNSSATVKLNTGAIIPQLGLGTWRSSDEEAYNSVLSAIKLGYRHIDSAAIYGNEEPVGKAIRDSGVPRNELFVTTKLWGTQQRNPEKALDASLKRLGLDYVDLYLIHWPIAFKTDNIKDDDYLTVPTGKDGKADVDVKDWNFVKTWELVQELPATGKAKAVGVSNFSVNNLKELLASPGNKLVPACNQVELHPLLPQDELIEFSKSKGIVIEAYSPLGSNGSPLLKEPVIIEVAKKYGVEPAQVLINWGIQRGYVILPKSVNPKRVESNFKVFDLSKEDVDRIHKISKEQGVKRFVHPDWPSFPIFE

SEQ ID NO: 54 - protein, 310 aa, Zygosaccharomyces rouxii
MVTLQNSTSTLKLNTGQTIPQVGLGTWRSKENEGYKAVIEALKAGYRHIDGAAVYGNEGEVGKAIQDSGVPRNEIFLTTKLWCTQQRNPQEALDQSLQRLGLDYVDLYLIHWPVPLRTENIKDGNLFQFAEKPDGSKDVDLEWNFIKTWELMQKLLESGKTKAIGVSNFSVNNLKDLLAAPTTKVTPAVNQVELHPLLPQEDLIKFGKENGIVIEAYSPLGGENAPILSDPTVQEIAKANGVEAGHVVISWAVQKGLVTLPKSVTPSRIVGNLKVLTLSDSDVAKVDGLLKAKGERRTCYQDFSPFPIFQ

SEQ ID NO: 55 - protein, 313 aa, Saccharomyces castellii
MTVTLKASTATLKLNTGANIPEISLGTWRSLGATDGYDSVIAALKAGYRHIDTAAIYGNEDQVGKAIRDSGIPREEIFVTSKLWNTQQHNPAGALKDTLSRLGLDYLDLYLMHWPVTFNTRNVKGTDFMVIPKNENGKPDIEMDTWNFVKTWELMQELPATGMTKAVGVSNFSINNLKELLTSPGNKLTPAANQIEIHPLLPEHDLIKFCREKGIMIEAYSPLGSINAPILKEPKIIEISKKLDVPPAQLIISWHVQRGYVVLVKSTHEERIIANRKVFTLSKEDFEAIKNLAKEKGTLRVVDPDWSPFPLFK

SEQ ID NO: 56 - protein, 312 aa, Saccharomyces bayanus
MSATLQYSTKTLPLNTGAKIPQVGLGTWQSKDNDAYKSVVAALKDGYRHIDTAAIYGNEDQVGQAIKDSGVPREEIFVTTKLWCTQHHEPAKALDQSLKRLGLDYVDLYLMHWPVRLDPNYIKDGHILSIPTKEDGSRPVDITNWNFIKTWELMQELPKTNKTRAVGVSNFSINNLKDLLASPGNQLTPAANQVELHPLLPQNELIDFCKSKGIMVEAYSPLGSTDAPLLKEPVVLEIAKKNNVQPGHVVISWHAQRGYVVLPKSVNPDRIKTNRKVFTLSDEDFEAINNISKEKGEKRVVNPDWSPFEAFK

SEQ ID NO: 57 - protein, 310 aa, Candida glabrata
MAALHKNTSTLKLNTGAHIPVIGLGTWQSSENEGYNATLEALKAGYRHIDTAAIYKNEEEIGRAIRDSNIPRNELFVTTKLWGTQHRNPTEALDQSLKRLGLDYVDLYLMHWPVALKTDLIKDGNLLQIPEREDGSRDVDLEDWNFVKTWELMQELPKEKARAIGVSNFSINNLKELLNSKGNKVVPAANQIEIHPLLPQDELINFCKEKGIVLEAYSPLGSTDAPILKEEEITEIAKKNGVNAGQLVISWHAQRGYVVLPKSVKPERIHGNQETFKLSDEDFATLSNYAKKHGERRVVSPNWGPFKPFV

SEQ ID NO: 58 - protein, 311 aa, Kluyveromyces polysporus
MSLKNCETRVKLNTGAELPAVGLGTWQSLENDAYKAVLTALKTGYRHIDGAAIYCNEGEVGKAIRDSGVPRNEIFVTTKLWGTQQRTPQLALEQSLERLGLDYVDLYLIHWPIPLNPAQCEESGNYLTFPGLPNGKRDVDLDTWNFVKTWELMQELPATGKCKAVGVSNFSINNIKELLSSPGNKLVPAVNQIELHPLLPQVELVNFCKEKGIVVEAYSPLGGTGAAILKEPLVEELAKKYNIPAANLVVSWDVQRGVVVLPKSVTESRIISNLQTLTLAPEDVEKITNLSKEKGEKRTCAPDFSPFNTFA

SEQ ID NO: 59 - protein, 312 aa, Saccharomyces bayanus
MPATLKNSSATLKLNTGASIPVLGFGTWRSVDNSGYHSVIAALKAGYRHIDAAAIYLNEEEVGKAIKDSGVPREEIFITTKLWGTEQRDPEAALDKSLKRLGLDYVDLYLMHWPVPLKTDTITDGDFLCIPTLEDGTIDVDTKEWNFVKTWDLMQKLPKTGKTKAVGVSNFSINNIKELLKSPSNKLVPATNQIEIHPLLPQDELIAFCKEKGIVVEAYSPFGSANAPLLKEQAIIDMAKKHGVEPAQLIISWSIQRGYVVLAKSVNPERVVSNFKIFTLPEDDFKTISNLSKVHGEKRVVNMQWNSFPIFE

SEQ ID NO: 60 - protein, 322 aa, Saccharomyces castellii
MPTSIAKHEKQMSASNSTTTFKLNTGATISAVGLGTWQSTNENEGYEAVIAALKAGYRHIDTAAIYGNEGQVGKAINDSGVPREEIFVTTKLWSTQHHDPAAGLNDSLKRLGLDYVDLYLMHWPVTLNPRSIKDGNFLSIPLLPDGKRDVEIDTWDYVKTWELMQELPKTGKTRAVGVSNFSINNLKKVLSSPGNKVVPAVNQVEIHPLLPQEELLEWCHSKGILLEAYSPLGSTNAPILTEPVLINLAKKHGVQTAQIVISWHVQRGYIVLPKSVHAERIQANFKTLKLSDEEMREINNISKEKGERRIVQPHWEPFVPFV

SEQ ID NO: 61 - protein, 310 aa, Candida glabrata
MSLKNSTATLKLNTGAEIPVVGLGTWRSAANDGYDSVLAALKLGYRHIDAAAIYGNEDQVGRAIKDSGVPRQEIFITTKLWGTEHRNPAKALDSSLKRLGLDYVDLYLMHWPVALKAHGSEEKDLLNIPKKPDGKTDIDIEDWDFIKTWELMQELPKTGKTKAIGVSNFSINNLKALKNSPKFNTVPAANQVEIHPLFPQTELIEYCKSENILIEAYSPFGSADAPVLKEPELHKLAEKYNISTAQLIISWSVQRGYVVLPKSVHAERIEANLKVLTLKDEDMETITNLSKVKGEKRLVQLDWSPFPTFE

SEQ ID NO: 62 - protein, 329 aa, Kluyveromyces thermotolerans
MVYVSETVKLNNGLEMPLVGLGCWKIPNEVCASQVYEAIKLGYRLFDGAQDYGNEKEVGQGIRKAIDEGIVKREDLFVVSKLWNSYHDPKNVKKALKRTLQDLGLEYLDLFYIHFPLAFKFVPFEERYPPGFYTGAEDEKKGVISLEKVTVLDTYRALEQCVDEGLIKAIGVSNFRGILLHDLLAGAKIPPACLQIEHHPYLTQEKLVEYCKQQGIQVVAYSSFGPQSFLEMDSQLAKSTPTLFEHDVVKKVANNHGVSTSEVLLRWATQRRVAIIPKSSKKERLLNNLQINEKLTLSDEELQEISSLNKDIRFNDPFNWLDMKFPVFA

SEQ ID NO: 63 - protein, 329 aa, Kluyveromyces waltii
MTYTSETVKLNNGLQMPLVGLGCWKIPNEVCASQVYEAIKLGYRLFDGAQDYGNEKEVGQGIHKAIDEGIVKREDLFVVSKLWNSYHDPKNVKKALKRTLEDMELEYLDLFYIHFPLAFKYVPFEEKYPPGFYTGAEDAKKGVISLEQVPIIDTYRALEECVKEGLIKSIGISNFRGILIHDLLAGATIKPVCLQIEHHPYLTQEKLIEYCAQEQIQVVAYSSFGPQSFLELDSELAKSTPTLFEHEGIQKIATNHGVSSSQVLLRWATQRRIAIIPKSSKKERLLDNLQINEKLTLTEAELQEISALNKDIRFNDPFTWLGMKFPVFA

SEQ ID NO: 64 - protein, 326 aa, Candida glabrata
MSSVVTLNNGLKMPLVGLGCWKIPNDVCAQQVYDAIKIGYRLFDGAEDYGNEKEVGQGIRKAIDEGVVKREDVFVVSKLWNSFHHPDHVKMALKRTLSDMGLDYLDLFYIHFPIAFKYVPFEEKYPPGFYTGKEDEKNGHITEAHVPIIDTYRALEQCVEEGLIKSIGISNFSGSLVQDLLRQCKIKPVALQVEHHPYLTQEHLVEYCKDNGIQVVAYSSFGPQSYIELNHPLAKNTPNLFHHDTIKQIANNHNVATSQVLLRWATQRGIAIIPKSSKKERLQDNLMIDEKLTLTHKELEAISKLNKNLRLNDPWDWLDGKFPIFA

SEQ ID NO: 65 - protein, 315 aa, Saccharomyces bayanus
MPLVGLGCWKIDNKVCANQIYEAIKLGYRLFDGACDYGNEKEVGEGIRKAISEGLVTRKDIFVTSKLWNNFHHPDHVKLALKKTLDDMGLDYLDLYYIHFPIAFKYVPFEEKYPPGFYTGAEDDKRGHITEAHVPIIDTYRALEACVDEGLIKSIGISNFQGSLVQDLLRGCRIKPVALQIEHHPYLTQEHLVDYCKLHDIQVVAYSSFGPQSFIEMDLGLAKSTPTLFENDVVKKVSENHPGSTTSQVLLRWATQRGIAVIPKSSKKERLLGNLEIEKKLTLTDQELKEISSLNANIRFNDPWTWLDGKFPIFA

SEQ ID NO: 66 - protein, 354 aa, Ashbya gossypii
MVYKQLYMRGTPPDLSRCITGQDIAMVMNVRTIKLNNGADMPLVGLGCWKIPNEVAAEQVYEAIKLGYRLLDGAEDYANEREVGQGIRRAIDEGLVRREELFVVSKLWNNYHRPEDVGKALQRTLSDLGLDYLDLFYIHFPLAFKFVPLEERYPAGLYTGAADEQAGRLAQEPVPLIETYRALEQLVDEGRIRAIGLSNFQGCLVQDLLRGCRIRPAALQIEHHPYLTQERLVQYAKSEGLAVVGYSSLGPQSFLELGNTAAQNAAPLLQHEVVCRIAERHTATPAEVLLRWATQRDIAVIPKSSRKERLATNLRVDEALTLADDELRELSSLNCGLRFNDPWDWSAQPLPTFI

SEQ ID NO: 67 - protein, 329 aa, Kluyveromyces lactis
MTYLAETVTLNNGEKMPLVGLGCWKMPNDVCADQIYEAIKIGYRLFDGAQDYANEKEVGQGVNRAIKEGLVKREDLVVVSKLWNSFHHPDNVPRALERTLSDLQLDYVDIFYIHFPLAFKPVPFDEKYPPGFYTGKEDEAKGHIEEEQVPLLDTWRALEKLVDQGKIKSLGISNFSGALIQDLLRGARIKPVALQIEHHPYLTQERLIKYVKNAGIQVVAYSSFGPVSFLELENKKALNTPTLFEHDTIKSIASKHKVTPQQVLLRWATQNGIAIIPKSSKKERLLDNLRINDALTLTDDELKQISGLNQNIRFNDPWEWLDNEFPTFI

SEQ ID NO: 68 - protein, 326 aa, Saccharomyces castellii
MPKLLTLNNGMKMPQVGLGCWKIPNDICADQVYEAIKAGYRLFDGATDYANEVEVGKGLKRAMDDGIVKRQDLFVVSKLWNNFHHPDHVKLNLKRNLKDLGLDYLDLYYIHFPIAFKFVPMEERYPPQMYTGKADEAKGILTEENVPLLDTYRALEECQREGLIKSIGISNFNGGLVADILRGCSIKPVALQIEHHPYLTQEKLLEYCKIHDVQVVGYSSFGPQSFADCGMDLAKNTPPLFDHPVIKKIAEKHNVSTAEVLLRWATQRGVAIIPKSSHKKRLLSNLNIDDNVTLTEEDLKSISGLNQNLRFNDPWDWNNSKFPIFH

SEQ ID NO: 69 - protein, 327 aa, Zygosaccharomyces rouxii
MASVVALNNGNKMPLVGLGCWKIPNETCSQQIYDAISVGYRVFDGAQDYGNEKEVGEGVRRAIKDGLVKREELFVVSKLWNSFHHPKNVKLALKRTLSDMGLDYLDLFYIHFPIALKPVSFEEKYPPGLYTGEADAKAGVLSEEPVPILDTYRALEECVEEGLIKSIGVSNFSGSIMLDLLRGARIPPAALQIELHPYLTQERYVKWVQSKGIQVVAYSSFGPQSFVDIGSEVAKATPPLFEHDVVKKIAAKHNVSTSQVLLRWATQQKVAVIPKSSKKERLRQNLLVDQEVTLTGDEIKEISGLNKNLRFNDPFTWSEKTPFPIFD

SEQ ID NO: 70 - protein, 329 aa, Saccharomyces kluyveri
MSALSKTVTLNNGLEMPLIGLGCWKIPNDVCASQVYEAIKLGYRLFDGAQDYGNEKEVGQGIKKAIAEGIVKREDLFVVSKLWNSFHHPDNVKLALKKTLDDIGLDYLDLFYIHFPLAFKFVPFEEKYPPGFYTGAEDEKNGVISQEDVPLIDTYRALEKCVDEGLIKSIGISNFNGSLVQDLLRGARIKPVALQIEHHPYLTQENLVKYCKNVGIQVVAYSSFGPQSFIELDSELAKTTPTLFRNDTVKLIANNHKVSTSQVLLRWATQRGVAVIPKSSKKERLLENLTINDSLTLSDKELKAISALNKDIRFNDPWTWLNSEFPTFA

SEQ ID NO: 71 - protein, 400 aa, Saccharomyces bayanus
MSFVKDFKPQALGDTNLFKPIKIGNNELLHRAVIPPLTRMRALHPGNVPNKDWAVEYYSQRAQRPGTMIITEGAFISPQAGGYDNAPGVWSEEQMAEWTKIFNAIHEKKSFVWVQLWVLGWAAFPDTLARDGLRYDSASDNVYMDAEQEAKAKKANNPQHSLTKDEIKQYIREYVQAAKNSIAAGADGVEIHSANGYLLNQFLDPHSNTRTDEYGGSIENRARFTLEVVDALVEAIGQEKVGVRLSPYGVFNSMSGGAETSIVAQYAYVLGELERRAKAGKRLAFVHLVEPRVTNPFLAEGEGEYNEGSNDFVYSIWKGPIIRAGNFALHPEVVREEVKDKRTLIGYGRFFISNPDLVDRLEKGLPLNKYDRNTFYQMSAHGYTDYPTYEEALKLGWDKK

SEQ ID NO: 72 - protein, 401 aa, Candida glabrata
MPFVKDFKPIALKDTNMFKPIKIGNTEVAHRVVMPPLTRMRAHHPGNVPNKEWAVEYYKQRSQRPGTMIITEGAFPCAQAGGYDNAPGIWSEEQVAEWTKIFKAIHDNKSFVWVQLWVLGRQAFPDTLARDGLRYDSASDEVYMDKESEEKAKKSNNPQHGITKDEIKQYIKDYVQAAKNSIAAGADGVEIHSANGYLLNQFLDPISNKRTDEYGGSIENRARFTLEVVDAIVDAIGPEKTGIRFSPYGTFGTMSGGAEPLIVAQYAYVLGELEKRAKAGKKLAFVHLVEPRVTNPFFTEGQGEYKDGTNDFAYSIWTGPIIRAGNYALHPEIAKEHVNNDDRTLIGYGRFFIANPDIVDRIEKGLPLNKYDRDTFYAMTTEGYLDYPTYDEAIKMGWDKQ

SEQ ID NO: 73 - protein, 400 aa, Saccharomyces castellii
MPFVKDFTPVALGDTNLFKPIKIGNNQLQHRVVLPPLTRMRATHPGNVPNKDWAVEYYNQRSQRPGTLLITEGAFICAQAGGYDNAPGIWSDEQVEQWKKIFAKIHENKSFVWVQLWALGRQSYADTLARDGLRYDSASDNVYMDAEQEERAKKANNPQHGLTKEEIKEYIKCYVHAAKNSIAAGADGVEIHSANGYLLNQFLDPISNKRTDEYGGSIENRARFTLEVVDALVDAIGEDKVGVRLSPYGTFGTMSGGADPTLLAQYAYVLGELEKRAKAGKRLAFVHLVEPRVTNPFLTEGQGEYNDGTNDFAYSIWKGAIIRAGNLALHPESVKQMVADDRTLIGYGRFFIANPDIVDRLEKGQQFNKYDRDTFYAMTAHGYTDYPTYDEAIKLGWDKQ

SEQ ID NO: 74 - protein, 396 aa, Saccharomyces bayanus
MPFVKDFEPVSLENTNLFKPIKLGNTELPHRVVMAPLTRMRASHPGNVPNKEWAAEYYAQRAQRPGTMIITEGAFISPQAGGYDNAPGIWSDEQVAQWRKILQAIHEKKSFAWVQLWALGWASFPDAMARDGLRYDSASDAVYMEGTAKAAKVPGHSLTKDDIEQYVKEYVHAGKNAVLAGADGVEIHSANGYLLNQFLDPRSNTRNDEYGGSIENRARFTLQVVDAVADAIGHDRVGLRLSPYGTFNDMSGGAEPDIVAQYAYVLGELEKRAKAGKRLAYVHLVEPRVTNPLLDEGEGEYDKGSNDFAYSIWKGPIIKAGNFALHPNSVKEEVKCDRTLIAYGRFFISNPDLVDRLEKGLPLNKYDRATFYTMSAQGYTDYPTYDESVDLGWDKN

SEQ ID NO: 75 - protein, 320 aa, Kluyveromyces polysporus
MAHNKVTIIGSGPAAHTAAIYLARAEIPPTLYEGMFANGIAAGGQLTTTTEIENFPGFPDALTGSELVERMKQQSTKFGTTVITETISKVDLSSRPFKLWTEFNEDAEPITTDALIIATGASAKRMHIPGEETYWQKGISACAVCDGAVPIFRNKPLAVIGGGDSACEEAEFLTKYGSKVYLLVRKDHLRASTIMQRRVEKNEKIEILYDTVALEAQGDSKLLQSLKIKNVKTDTESELPVNGLFYAIGHTPATQIVKGQIDQDEVGYIKTIPGTSETSVPGVFAAGDVQDSRYRQAITSAASGCKAGLDAEKYLGSLEE

SEQ ID NO: 76 - protein, 330 aa, Saccharomyces kluyveri
MRKQILQIPKRMVHHRVTIIGSGPAAHTAAIYLARAEIKPTLYEGMFANGIAAGGQLTTTTEIENFPGFPEGLTGSELMERMKQQSVKFGTEVITETVSKVDVSSKPFKFWTEFNEDSEPETTDAIILATGASAKRLNLPGEETYWQQGISACAVCDGAVPIFRNKPLAVVGGGDSACEEASFLTKYGSKVYLLVRKDHLRASTIMQRRAEKNEKIEILYNTVTLEAKGDGKFLNALRIKNVKTNEESDLQVNGLFYAIGHTPATKILEGQVDLDEAGYVKTVPGSSLTSVPGVFASGDVQDSRYRQAITSAGSGCMAGLDAEKYLSELE

SEQ ID NO: 77 - protein, 340 aa, Kluyveromyces waltii
MYLKPFTRSKGSFVQARQFRKMAHHKVTIIGSGPAAHTAAIYLARAEIKPTLYEGMFANGVAAGGQLTTTTEIENFSGFPEGLTGSELMDRMKQQSEKFGTDIITETVAKVDVTSRPFKLWTEFNEDAEPITTDAIILATGASAKRLNLPGEETYWQQGISACAVCDGAVPIFRNKPLAVIGGGDSACEEACFLTKYGSKVYMLVRKDHLRASTIMQRRAEKNDKIEILYNTVSLEAKGDGKFLNALKIKNVKTNEESDLPVNGLFYAVGHTPATSLLAGQVDLHENGYVKTVPGSSLTSVPGVFAAGDVQDSRYRQAITSAGSGCMAALDAEKFLAEQE

SEQ ID NO: 78 - protein, 319 aa, Candida glabrata
MNHKKVVIIGSGPAAHTAAIYLARAEIKPTMYEGMLANGIAAGGQLTTTTEIENFPGFPDGMTGSELMDRMRAQSTKFGTEIITETIAKVDLSSRPFKLWTEFNEDGEPITTDAIVIATGASAKRLHIPGEETYWQQGISACAVCDGAVPIFRNKPLAVIGGGDSACEEAQFLTKYGSKVYLIVRKDHLRASTIMQRRAEQNDKIEILYNTVTLEAQGDGKLLNNLRIKNVKTNEETDLPVNGLFYAIGHTPATKIVEGQVETDETGYIKTIPGSSLTSVPGVFAAGDVQDSKYRQAITSAGSGCMAGLDAEKYLTELE

SEQ ID NO: 79 - protein, 319 aa, Saccharomyces bayanus
MVHNKVTIIGSGPAAHTAAIYLARAEIKPTLYEGMMANGVAAGGQLTTTTEIENFPGFPEGLTGSELMDKMRLQSTKFGTEIITETVSKVDLSSKPFKFWTEFNEDEEPITTDAIILATGASAKRMHLPGEETYWQKGISACAVCDGAVPIFRNKPLAVIGGGDSACEEAQFLTKYGSKVFMLVRKDHLRASTIMQRRSEQNEKIEILYNTVALEAKGNGKLLNALRIKNTQSNEETDLPVNGLFYAIGHTPATKIVADQVNVDEAGYIKTVPGSSLTSVPGFFAAGDVQDSKYRQAITSAGSGCMAALDAEKYLSSLE

SEQ ID NO: 80 - protein, 336 aa, Ashbya gossypii
MLAAATRYRLGQALQTRMVHHKVTIIGSGPAAHTAAIYLARAEIKPTLYEGMFANGVAAGGQLTTTTEIENFPGFPDGLTGSDLMERMKAQSVKFGTEVVTETVAKVDLSARPFKLWTEFNEDEEPTTTDAIILATGASAKRLGLPGEETYWQRGISACAVCDGAVPIFRNKPLAVVGGGDSACEEASFLTKYGSKVFMLVRKDHMRASTIMQRRVERNEKIEVLYNTAPVEAKGDGSLLDALRVRDTRTGEESDLPVNGLFYAIGHTPATQLVAGQVDLDESGYVKTVPGSTLTNVPGLFAAGDVQDSRYRQAVTSAGSGCMAALDAEKFLSELE

SEQ ID NO: 81 - protein, 319 aa, Candida glabrata
MPHNRVTIIGSGPAAHTAAIYLARAEMKPTLYEGMLANGIAAGGQLTTTTEIENFPGFPEGLTGTELMDRMRAQSTKFGTNIITETISKVDLSAKPFKLWTEFNEDGEPITTDAIVMATGASAKRLDLPGEDKYWQQGISACAVCDGAVPIFRNKPLAVVGGGDSACEEAEFLTKYGSKVFMIVRKDHLRASTIMQRRVEKNEKIEVLFNSVSLEAKGDGKFLNALRIKNVKTNEESDLPVNGLFYAIGHNPATDIVKGQVETDESGYIKTVPGSSLTSIPGFFAAGDVQDSRYRQAITSAGSGCMAALDAERYLTELE

SEQ ID NO: 82 - protein, 339 aa, Saccharomyces castellii
MKGTQLHHPLIHIHHSNNDKMVHHKVTIIGSGPAAHTAAIYLARAEIKPTLYEGMMANGIAAGGQLTTTTEIENFPGFPDALTGSELMDRMRAQSVKFGTDIITETISKVDLSSKPFKLWTEFNEDEAPVTTDAIVLATGASAKRLHLPGEETYWQQGISACAVCDGAVPIFRNKPLAVIGGGDSACEEAQFLTKYGSKVYMIVRKDHLRASTIMQRRAEKNAKIEILFNTVTLEAKGDGKFLNALRIKNVKDEKESDLAVNGLFYAVGHTPATKIVEGQVDTDSAGYIKTVPGSSLTSVPGFFAAGDVQDSRYRQAITSAGSGCMAALDAERYLTELE

SEQ ID NO: 83 - protein, 319 aa, Kluyveromyces polysporus
MVHNKVTIIGSGPAAHTAAIYLARAELPPTLYEGMFANGIAAGGQLTTTTEIENFPGFPDALTGSELVERMKEQSVKFGTKVITETISKVDLSSRPFKLWTEFNEDAEPVTTDALIIATGASAKRMHIPGEDSYWQKGISACAVCDGAVPIFREKPLAVIGGGDSACEEAEFLTKYGSKVFMLVRKDHLRASTIMQRRVERNEKIEILYNTVALEAQGDGKLLQSLKIKNVNTNEESELPVNGLFYAIGHTPATSIVKGQVDQDDVGYIKTVPGTSLTSVPGFFAAGDVQDSRYRQAITSAGSGCMAALDAEKYLAELE

SEQ ID NO: 84 - protein, 341 aa, Saccharomyces bayanus
MIRHIAPLRTYLGRVGTRVQSRMVHHKVTIIGSGPAAHTAAIYLARAEMKPTLYEGMMANGVAAGGQLTTTTEIENFPGFPEGLTGSELMDRMRQQSARFGTNIVTETISKVDLSSKPFKLWSEFSEDADPVTTDAIILATGASAKRMNLPGEETYWQKGISACAVCDGAVPIFRNKPLAVIGGGDSACEEAEFLTKYGSKVYILVRKDHFRASVIMQRRIEKNSKIIVLFNTIALEAKGDGKLLDVLRIKNTKTGVENDLEVNGLFYAIGHNPATEIVNGQVDKQETGYIKTVPGSSLTSVLGFFAAGDVQDSRYRQAVTSAGSGCIAALDAERYLSAQE

SEQ ID NO: 85 - protein, 345 aa, Zygosaccharomyces rouxii
MLVNSGRTLFSRASSTTSTTNTIIKRMVHNKVTIIGSGPAAHTAAIYLARAEIKPTLYEGMMANGIAAGGQLTTTTDIENFPGFPQGLLGSELMDRMKEQSVKFGTEVITETISKVDLSSKPFKFWTEFNEDGPANTTDAMILATGASARRVNLPGEETYWQQGISACAVCDGAVPIFRNKPLAVIGGGDSACEEAGFLTKYGSKVYLLVRKDHLRASTIMQRRVQKNDKIEILYNTVAVEAKGDGKLLNTLRIKNVKENQESDLQVNGLFYAIGHTPATSIVQGQVTTDESGYIKTIPGTAETNVPGFFAAGDVQDSKYRQAITSAGTGCMAGLDAERYLSSLE

SEQ ID NO: 86 - protein, 340 aa, Kluyveromyces thermotolerans
MLLKPLTRSFRSSPLFQSPKRMVHHRVTIIGSGPAAHTAAIYLARAEIKPTMYEGMFANGVAAGGQLTTTTDIENFPGFPEGMSGSELMDRMKKQSERFGTDIITETVAKVDVTSRPFKFWTEFNEDAEPNTTDALILATGASAKRMNLPGEETYWQQGISACAVCDGAVPIFRNKPLAVIGGGDSACEEACFLTKYGSKIYMLVRKDHLRASTIMQRRAEKNEKIEILYNTVALEAKGDGKFLNALRIKNVQTNEESDLPVNGLFYAIGHTPATSLLAGQVDLHENGYVKTVPGSALTSVPGVFAAGDVQDSRYRQAVTSAGSGCMAALDAEKFLAEQE

SEQ ID NO: 87 - protein, 349 aa, Kluyveromyces lactis
MLLVRNSTLGRLSSLRGFFRNINESNIFYRMVHHKVTIIGSGPAAHTAAIYLARAEIKPTLYEGFMANGIAAGGQLTTTTEIENFPGFPDGLTGSELMDRMKAQSIKFGTDVITETVSKVDLSSRPFKFWTEFNEDQEPETTDAIILSTGASAKRLHLPGEETYWQQGISACAVCDGAVPIFRNKPLAVIGGGDSACEEAQFLTKYGSKVYMLVRKDHLRASQIMQRRAEQNEKIEILYNHVTLEAKGDGKYLNALKVKNVKTNEEYDLPVNGLFYAIGHTPATNIVAGQVDLDEAGYVKTVPGSTLTNVPGVFAAGDVQDARYRQAITSAGSGCMAALDAEKYITELE

SEQ ID NO: 88 - protein, 348 aa, Saccharomyces castellii
MNIIIKQQGLLKTPLSLQKNNTFKLLLNRMVHHKVTIIGSGPAAHTAAIYLARAEIKPTLYEGMMANGIAAGGQLTTTTEIENFPGFPDGLTGSELMDRMRAQSEKFGTEIITETISKVDLSSRPFKIWTEFNEDEEPITTDAIVLSTGASAKRLHLPGEETYWQKGISACAVCDGAVPIFRNKPLAVIGGGDSACEEAQFLTKYGSKVYLIVRKDHLRASTIMQRRAEKNEKIEIVYNTVTLEAKGNEKLLNGLRVKNVKTNEESDLEVSGLFYAIGHTPATKIVEGQVDLDESGYIKTVPGSSLTSVPGLFAAGDVQDSRYRQAITSAGSGCMAGLDAERYLTELE

SEQ ID NO: 89 - protein, 312 aa, Saccharomyces kluyveri; identical to SEQ ID NO: 53

SEQ ID NO: 90 - protein, 310 aa, Zygosaccharomyces rouxii; identical to SEQ ID NO: 54

SEQ ID NO: 91 - protein, 313 aa, Ashbya gossypii
MSTKATLKASTDTVTLNTGAKIPLFGLGTWQSTDDDASSAVAAALKHGYKHIDTASFYKNEELVGKAVKESGIPREELFITTKVWNDQQRDPAGALDLSLKKLGTEYVDLLLMHWPVPFKEPENSTLDAYKVPRGPDGKVTRDEEWDFVKTWHLMQKLLGTGKVKAIGVSNFSINNLEELLNAEGTTVVPAVNQVELHIRLPQLELVEYCQKKGIVVEAYSPLGSSSAPLLKDATVNKVAEKYGVTPAHVLLNYVANRGIVVLPKSVKESRIISNFEYFKMDEEDIKLLNDIHKKEGVQRFVDPDFSPFPLFQ

SEQ ID NO: 92 - protein, 310 aa, Candida glabrata; identical to SEQ ID NO: 57

SEQ ID NO: 93 - protein, 312 aa, Saccharomyces bayanus; identical to SEQ ID NO: 56

SEQ ID NO: 94 - protein, 313 aa, Saccharomyces castellii; identical to SEQ ID NO: 55

SEQ ID NO: 95 - protein, 309 aa, Kluyveromyces thermotolerans
MTTVLKKCTATAKLNTGAQIPLLGLGTWRSTKENGYNATLAAIKAGYRHIDIAAVCMNEQVVGRAIRDSGVPREELFVTTKLWCTQHRDPKTALSQSLKRLGLDYVDLYMMHWPVAFRSEQVKHTNYMLIPKRKDGQPDVDEGWDYVKTWELMQELPATGKTNALGVANFSVSQLKKLIDSPGNKIVPAASQLETHPFLPQNKMLSFCKKNNILMEAYSPLGSEGAPLLDEPVVREISKKLHVEPAQLLVSWGLQRGHAVLVKSKTPSRIEANLHTFDIPEGDLRRINELAQERGTKRIHNPTWFSFEE

SEQ ID NO: 96 - protein, 311 aa, Kluyveromyces polysporus; identical to SEQ ID NO: 58

SEQ ID NO: 97 - protein, 308 aa, Kluyveromyces waltii
MTGILKKCTATATLNTGAKIPLLGLGTWRLTKEGGYKAILAALKAGYRHIDSAAVCMNEQVIGKAIRDSGVRRDELFVTTKLWSTQHRDPSAALDQSLKRLGLEYVDLYMMHWPLAFRVDQIKAKNFMLIPRNSEGKPYVDTDWDYVKTWELMQKLPTEKTRAIGVANFSVPQLTSLLKSPNNKIIPAASQLETHPFLPHDEMLAFCREKGILMEAYSPLGGDGGPLIDEPVIREISKKLHVEPAQLLISWGLKRGCAVLPKSKTQTRIESNLEVFDLSDEDYERIHEIAQERGPKRTHNPPWFSFEE

SEQ ID NO: 98 - protein, 312 aa, Saccharomyces bayanus; identical to SEQ ID NO: 59

SEQ ID NO: 99 - protein, 322 aa, Saccharomyces castellii; identical to SEQ ID NO: 60

SEQ ID NO: 100 - protein, 310 aa, Candida glabrata; identical to SEQ ID NO: 61

SEQ ID NO: 101 - protein, 497 aa, Kluyveromyces lactis
MATQFDENTVITIFGASGDLSKKKTFPALFGLYREGYLNPTTKIIGYARSKLSNEDLREKVKPFLKKPNGAKDDAKVNEFLSMVSYHAGPYDSDEGYLELKKIIEEFEAEKKVDEPHRLFYLALPPSIFIDVCSKLKENLYTESGIQRVIVEKPFGHDLQSATELQEKLAPLFSEDELFRIDHYLGKEMVKNLLLMRFGNTFLNAAWNKENIQSVQVVFKEPFGTEGRGGYFDSIGIIRDVMQNHLLQVLTLLTMERPVSFDPESVRDEKVKVLKAFSPIDHDDILIGQYGRSVDGSKPSYLDDETVKEDSKCVTFAAIGFKIANERWDGVPIVMRAGKALNEGKVEIRIQFRRVASGMFTDIPNNELVIRIQPNEAIYLKCNAKTPGLANENQTTELDLTYSERYKNYWIPEAYESLIRDALLGDHSNFVRDDELDVSWKLFTPLLNYLEGPDGPQPKIYPYGCRSPDGLVEFLADHGYTFSKPGSYQWPVTTPKM

SEQ ID NO: 102 - protein, 512 aa, Saccharomyces castellii
MRAFDIPAAATCPVKFVKDTVIVVFGASGDLAKKKTFPALFGLFREGYLDPSTKIIGYARSQLTHSELRAHIEPHLGKPNGKEVDDPKAEQFFQMVSYISGAYDKDEGYVTLRDHIEQFEHERDVETPHRLFYFALPPNVFLSVAKQIKKLVYAENGLTRVIVEKPFGRDLESARKLQNELGPLFSEEEIYRIDHYLGKELVKNLTMLRFGNQFLNASWNKENLQSVQISFKEPFGTEGRGGYFDNIGIIRDVMQNHLLQILTLVTMERPQSFDAEAIRDEKVKVLKAVQPIDVNNILVGQYGKSEDGTKPSYLDDETVDPNSKCITFAALNFNIDNERWRGVPIMMRAGKALNEGKVEIRLQFKKSYGVFADIPNNELVIRVQPNAAVYMKFNAKTPGLSNESQVTDLDLTYSSRYKNFWIPEAYEVLIRDALLGDHSNFVRDDELDVSWRLFTPLLNYLEGPDAPTPEIYPYGTRSPAGLREYMKKHNYVLHSKNNSYSWPVTTPTTSPK

SEQ ID NO: 103 - protein, 507 aa, Saccharomyces kluyveri
MSQPVAFEENTVITVFGASGDLSKKKTFPALFGLFREGYLHPSTKIIGYARSKLSNDDLKEKIKPHLKKPNGAEDDQKVEKFLSLVSYVAGPYDSDEGYLELKEAIELYEKENSVQEPHRLFYLALPPSVFVTVANQIKKLVYAANGITRVIVEKPFGHDLESSRELQEQLSPLFSEAELFRIDHYLGKEMVKNLLLIRFGNTFLNAAWNKENIQMMQISFKEPFGTEGRGGYFDEIGIIRDVMQNHLLQVLTLLTMERPVSFDPESVRDEKVKVLKAFTPIDHDDILTGQYGKSEDGSKPSYLDDETVKNDSKCVTFAALGFKIQNERWDGVPIVMRAGKALNEGKVEIRIQFKAVASGMFNNIPNNELVIRIQPNEAIYLKCNAKTPGLSTSTQLTELDLTYSSRYKNYWIPEAYESLIRDALLGDHSNFVRDDELDVSWKLFTPLLNYLEGPDAPQPEIYPYGSRGPKNLTKYLEEHKYVFQEEGLYQWPVASPSSSACSSPKM

SEQ ID NO: 104 - protein, 512 aa, Ashbya gossypii
MMCGEVTRTDAFEKDVIITVFGASGDLAKKKTFPALYGLYREGYLDDSTKIIGYARSELSDAELRERVKPYLSKPDDAASEAKQAEFLERMSYVAGPYDKEEGYVQLREQIEAYERERGVEKPHRLFYLALPPSAFVSVAEKIKQFLYAEGGVTRLIVEKPFGHDLESSRKLQKQLEPLFREEEIFRIDHYLGKEMVKNLLPLRFGNAFFNASWNKENIQMIQISLKEPFGTEGRGGYFDSTGIIRDVMQNHLLQVLTLLTMERPVSFDPESIRDEKVKVLKAIVPLDHEDILIGQYGRSEDGTKPAYLDDETVSKDSKCVTFAALTFNIQNERWDGVPIVMRAGKALNEGKVEIRILYKRVARGIFNDIPNNELVVRVQPDEAIYLKFNAKTPGLDSNSQITELDLTYSKRYRDYWIPEAYESLLRDALLGDHSNFVRDDELDISWKLFTPLLNYLEGPDSPRPEIYKYGSRGPDGLTKYLEKHQYVFEKSGLYQWPVSSPKEKASLEPRL

SEQ ID NO: 105 - protein, 509 aa, Kluyveromyces polysporus
MPFENSRMLQEKFEKDTVVTVFGASGDLAKKKTFPALFGLFREGFLDPSTKIFGFARSKLTDAQLRQHIEPYLQKPNGKDDDAKVEAFFKMVTYHSGQYDADEGYLSLRKLIEDFEKQRNITKPHRLFYFALPPSVFLSVARQIKKLVYADNGITRVIVEKPFGHDLKSARELQKNLAPLFTEDEIYRIDHYLGKEMVKNLLQLRFGNTFLNASWNKENIQSIQISFKEPFGTEGRGGYFDSIGIIRDVMQNHLLQVLTLVTMDRPVSFDPESVRDEKVKVLKAMAPIDMKDVLVGQYGKSEDGTKPSYLDDETVNPNSKCVTFAAMCFNIQNERWDGVPIIMRAGKALNEAKVEIRIQYKRVASGMFKNIPNNELVLRVQPDASVYVKFNSKTPGLSNATQVTDLDLTYSSRYKDFWIPEAYEVLIRDCMLGDHSNFVRDDELDVSWGLFTPLLEYLESPNAPQPEIYPYGSRGPKELKQYLSDHKFVFLSRHKYTWPVTSPEYDKDL

SEQ ID NO: 106 - protein, 500 aa, Candida glabrata
MSEPVKFEKNTVVVVFGASGDLAKKKTFPALFGLFREGYLHESTKIIGYARSSLTVEELTERIKPHLKTPRGKEDEAKIPQFFKLLTYVSGPYDQDDGFIRLREQIETFEGAKCSTVPHRLFYFALPPSVFLPVATNIKRLVYAENGVTRLIVEKPFGRDLETARKLQADLSPLFKEEEIYRIDHYLGKELVKNLLVMRFGNQFLNSAWNRDNIQSIQISFKEPFGTEGRGGYFDNIGIIRDVMQNHLLQVLTLLTMERPVSFDPEAIRDEKVKVLKAMAPFDPKDILIGQYGKSEDGSKPAYLDDETVKPGSKCVTFAAIAFQIENERWEGVPIVMRAGKALNEGKVEIRLQYKAVASGVFKNIPHNELVIRIQPNEAVYMKFNAKTPGLSNATQVTDLDLTYSSRYKDFWIPEAYEALIRDALLGDHSNFVRDDELDVSWSLFTPLLNYLEGPDAPQPEIYPYGSRGPSGLRDYLKKHNYMIEEKHPYTWPVTTPDED

SEQ ID NO: 107 - protein, 511 aa, Kluyveromyces thermotolerans
MSKPTEFKENTSIVVFGASGDLSKKKTFPALFGLFREGYLDPSCKIIGFARSNLSDEDLHEKIKPNLKTNNDEKGSEKIEQFLKMVSYISGPYDKDEGFDKLAKELDSYEKKQGVKSPHRLFYLALPPSAFVTVATQIKKKVYAEHGETRVVVEKPFGHDLDSFRELQSDLSPLFREDELFRIDHYLGKEMVKNLVFMRFGNTFLNAAWNKENIQMIQISFKEPFGTEGRGGYFDEIGIIRDVMQNHLLQVLTLLTMERPVTFDSEAVRDEKVKVLKAFAPIDHKDILIGQYGKSEDGSKPGYLDDDTVNPDSKCVTFAALGFKIQNERWDGVPIVMRAGKALNEGKVEIRIQFKAVPSGVFSEVAHNELVIRVQPDEAIYIKCNSKTPGLSTTSQVSELDLTYARRFKDFWIPEAYEALIKDVLSGDHSNFVRDDELDISWKLFTPLLKYLEGPDAPKPEEYAYGSRGPAELVNFLKSHDYVFEDSGSYQWPVITPEVTKKKQNSTDSKM

SEQ ID NO: 108 - protein, 507 aa, Kluyveromyces waltii
MSRPVEFKEDTSIVVFGASGDLSKKKTFPALFGLFREGYLHPTCKIVGFARSKLSDEDLHQKIEPNLKRSKDEQGEAKVREFLKMVSYISGPYDTDEGYEKLRTELETYEKKQGVKNPHRLFYLALPPSAFVTVASQLKKNVYAENGQTRIVVEKPFGHDLESFRKLQSELAPLFREDELFRIDHYLGKEMVKNLVLMRFGNTFLNAAWNKENIQMIQISFKEPFGTEGRGGYFDEIGIIRDVMQNHLLQVLTLLTMERPVSFDAESVRDEKVRVLKAFAPIDHDDILLGQYGKSEDGTKPGYLDDDTVNPDSKCVTYAALGFKINNERWDGVPIVMRAGKALNEGKVEIRIQFKAVPSGVFSDVAHNELVIRVQPNEAIYIKCNSKTPGLSTSSQVSELDLTYARRFKDFWIPEAYEALIKDVLSGDHSNFVRDDELDISWKLFTPLLQYLEGPNAPQPEEYAYGSRGPKRLVKFLEDHKYVFEDSGSYQWPVATPETTKDNSSKM

SEQ ID NO: 109 - protein, 513 aa, Zygosaccharomyces rouxii
MSVKYAKDTSIVVFGASGDLAKKKTFPALFGLFREGHLDATTRIYGYARSKLTKEELKERILPNLKRPSGAEGDAKVHKFFTMLHYVHGPYDTPEGYVKLKEVIEQHEEEVGVKEPHRLFYFALPPGVFLSVATEIKKNLYVPNGGTRLIVEKPFGHDLKSSRELQENLAPLFSEDEIYRIDHYLGKELVKNLLVLRFGNTVLNAAWNKENLQSIVISFKEPFGTEGRGGYFDSIGIIRDIMQNHLLQVLTLVTMERPVSFDAESVRDEKVKVLKAFAPIDHKDLLVGQYAKSADGSKPGYKDDETVDPKSKQLTFAALPFRINNERWDGVPIIMRAGKALNEGKVEIRLQYKDVASGVFQDIPQNELVIRIQPDPAVYMKFNAKTPGLSNVAQVTDLDLTYSSRYKDFWIPEAYEVLIKDALEGDHSNFVRDDELDVSWKLFTPLLEYLEGPEAPPPAEYPYGSRGPPGLREYMEKHGYTIAEQHNHPYTWPVTSPGSKERVRRASYAGKRW

SEQ ID NO: 110 - protein, 342 aa, Saccharomyces bayanus
MGLAKQVRLGDSGLKISPIVIGCMSYGSKKWADWVIEDKAQIFKILKHCYDNGLRTFDTADFYSNGLSERIIKEFLDHYSIKRETVVIMTKIFLPIDETLDLRHKFTLGEAEELDLTNQRGLSRKHIIAGVKNSVERLGTYIDLLQIHRLDHDTPMKEIMRALNDVVEAGDVRYIGASSMLATEFAELQFIADKYGWFQFISSQSYYNLLNREDERELIPFAKRHNIGLLPWSPNARGVLTRPLDKTTDRQKSDPTFKSLKLDALEDDQMEIVNRVEKISKEKNVSMAMISIAWVLHKGCHPIVGLNSTSRVDEAIAALQLTLTEEDVKYLEEPYKPKMQVG

SEQ ID NO: 111 - protein, 343 aa, Saccharomyces castellii
MSLVKQVRLGQSGLKISPILIGCMSYGSKKWADWVMEDKDEIFKILKHCYDRGLRTFDTADVYSNGISERLVGEFLKKYNIKRETVVIMTKIFFPVDESLDLHHGSNVNQEAMELQLTLQRGLSRKHIIDGVANSVERLGTYIDVLQIHRLDEETSMEEIMRSLNDVVMSGQVRYIGASSMLATEFAELQFIAEKNGWFKFISAQSYYNLINREDERELIPFCKKHGVGLIPWSPNARGVLTRPVEKNTERMLTDPTFNRMRLNELGDDEKEIVRRIQMVAERRSVTMAIISMAWTLHKGCNPIVGLNSVERVDEAVKAIEFTLTEDEIKLLEEPYKPKVRIF

SEQ ID NO: 112 - protein, 342 aa, Saccharomyces kluyveri
MTLPKPVRLGSSGLKISPIIVGCMSYGSKAWAPWVLDNEEEIFKVLKHCYDNGLRTFDTADFYSNGLSERLVGKFLKRYNINRETVVILTKVFFPVDETLDLSHSKGMDEATQLTVANQRGLSRKHILDGVAKSVERLGTYIDVLQIHRFDTETPVNETMKALNDVVSKGYARYIGASSMLATQFAELQFVAEKHDWFKFISSQSCYNLLYREDERELIPFAKRHNIGLIPWSPNARGVLTRPLGTQTLRHSTDFGLKRISAFDLQPHEEEIVRRVGEIAEKRGVTMAVVSIAWVLSKGCYPIVGLSSTERVDETIKAVEFKLTEEEATYLEEPYRPRAYLS

SEQ ID NO: 113 - protein, 267 aa, Saccharomyces cerevisiae
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 114 - protein, 267 aa, Vanderwaltozyma polyspora
MSQGRKASERLAGKTVLITGASSGIGKATALEYLDASNGHMKLILVARRLEKLQELKETICKEYPESKVHVEELDISDINRIPEFIAKLPEEFKDIDILINNAGKALGSDTIGNIENEDIKGMFETNVFGLICLTQAVLPIFKAKNGGDIVNLGSIAGIEAYPTGSIYCATKFAVKAFTESLRKELINTKIRVIEIAPGMVNTEFSVIRYKGDQEKADKVYENTTPLYADDIADLIVYTTSRKSNTVIADVLVFPTCQASASHIYRG

SEQ ID NO: 115 - protein, 267 aa, Saccharomyces castellii
MSQGPKAAERLNEKIVFITGASAGIGQATALEYMDASNGTVKLVLVARRLEKLQQLKEVIEAKYPKSKVYIGKLDVTELETIQPFLDNLPEEFKDIDILINNAGKALGSDRVGDIDIKDVKGMMDTNVLGLINVTQAVLHIFQKKNSGDIVNLGSVAGRDAYPTGSIYCASKFAVRAFTESLRRELINTKIRVILIAPGIVETEFSVVRYKGDNERAKSVYDGVHPLEADDVADLIVYTTSRKQNTVIADTLIFPTSQGSAFHVHRD

SEQ ID NO: 116 - protein, 268 aa, Candida glabrata
MSQGRKAAERLQGKIAFITGASAGIGKATAIEYLDASNGSVKLVLGARRMEKLEELKKELLAQYPDAKIHIGKLDVTDFENVKQFLADLPEEFKDIDILINNAGKALGSDKVGDIDPEDIAGMVNTNVLALINLTQLLLPLFKKKNSGDIVNLGSIAGRDAYPTGAIYCATKHAVRAFTQSLRKELINTDIRVIEIAPGMVETEFSVVRYKGDKSKADDVYRGTTPLYADDIADLIVYSTSRKPNMVVADVLVFPTHQASASHIYRGD

SEQ ID NO: 117 - protein, 267 aa, Saccharomyces bayanus
MSQGRKAAERLANKTVLITGASAGIGKATALEYLEASNGNMKLILAARRLEKLEELKKTIDEEFPNAKVHVGQLDITQAEKIKPFIENLPEAFKDIDILINNAGKALGSERVGEIATQDIQDVFDTNVTALINVTQAVLPIFQAKNSGDIVNLGSVAGRDAYPTGSIYCASKFAVGAFTDSLRKELINTKIRVILIAPGLVETEFSLVRYRGNEEQAKNVYKDTTPLMADDVADLIVYSTSRKQNTVVADTLIFPTNQASPYHIFRG

SEQ ID NO: 118 - protein, 273 aa, Zygosaccharomyces rouxii
MSQGVKAAERLAGKTVFITGASAGIGQATAKEYLDASNGQIKLILAARRLEKLHEFKEQTTKSYPSAQVHIGKLDVTAIDTIKPFLDKLPKEFQDIDILINNAGKALGTDKVGDIADEDVEGMFDTNVLGLIKVTQAVLPIFKRKNSGDVVNISSVAGREAYPGGSIYCATKHAVKAFTESLRKELVDTKIRVMSIDPGNVETEFSMVRFRGDTEKAKKVYQDTVPLYADDIADLIVYATSRKQNTVIADTLIFSSNQASPYHLYRGSQDKTN

SEQ ID NO: 119 - protein, 268 aa, Kluyveromyces lactis
MSQGRKAAERLQNKTIFITGASAGIGQATALEYLDAANGNVKLILAARRLAKLEELKEKINAEYPQAKVYIGQLDVTETEKIQPFIDNLPEEFKDIDILINNAGKALGSDVVGTISSEDIKGMIDTNVVALINVTQAVLPIFKAKNSGDIVNLGSVAGRDAYPTGSIYCASKHAVRAFTQSLRKELINTGIRVIEIAPGNVETEFSLVRYKGDADRAKQVYKGTTPLYADDIADLIVYATSRKPNTVIADVLVFASNQASPYHIYRGE

SEQ ID NO: 120 - protein, 267 aa, Ashbya gossypii
MSLGRKAAERLANKIVLVTGASAGIGRATAINYADATDGAIKLILVARRAEKLTSLKQEIESKYPNAKIHVGQLDVTQLDQIRPFLEGLPEEFRDIDILINNAGKALGTERVGEISMDDIQEVFNTNVIGLVHLTQEVLPIMKAKNSGDIVNVGSIAGREAYPGGSIYCATKHAVKAFTRAMRKELISTKIRVFEIAPGSVETEFSMVRMRGNEENAKKVYQGFEPLDGDDIADTIVYATSRRSNTVVAEMVVYPSAQGSLYDTHRN

SEQ ID NO: 121 - protein, 268 aa, Saccharomyces kluyveri
MSQGRRAAERLANKTVFITGASAGIGQATALEYCDASNGKINLVLSARRLEKLQELKDKITKEYPEAKVYIGVLDVTETEKIKPFLDGLPEEFKDIDILINNAGKALGSDPVGTIKTEDIEGMINTNVLALINITQAVLPIFKAKNFGDIVNLGSVAGRDAYPTGAIYCASKHAVRAFTQSLRKELVNTNIRVIEIAPGNVETEFSLVRYKGDTDRAKKVYEGTNPLYADDIADLIVYATSRKPNTVIADVLVFASNQASPYHIYRGD

SEQ ID NO: 122 - protein, 267 aa, Kluyveromyces thermotolerans
MSQGRRAAERLAGKTVFITGASAGIGQATAQEYLEASEGKIKLILAARRLDKLEEIKAKVSKDFPEAQVHIGQLDVTQTDKIQPFVDNLPEEFKDIDILINNAGKALGSDPVGTIDPNDIQGMIQTNVIGLINVTQAVLPIFKAKNSGDIVNLGSVAGREAYPTGSIYCATKHAVRAFTQSLRKELINTNIRVIEVAPGNVETEFSLVRYKGDSEKAKKVYEGTQPLYADDIADLIVYATSRKPNTVIADVLVFASNQASPYHIYRG

SEQ ID NO: 123 - protein, 267 aa, Kluyveromyces waltii
MSQGRKASERLAGKTVLITGASAGIGQATALEYLDASNGNIKLILAARRLEKLKEIKSQFEKDFPEAKVYIGQLDVTHTDEIKPFIDNLPEEFKDIDILINNAGKALGSDPVGTIDASDIEGMIQTNVVALINMTQAVLPIFKAKNAGDIVNLGSVAGREAYPTGSIYCATKHAVRAFTQSLRKELINTNIRVIEIAPGNVETEFSLVRYKGDPEKAKKVYEGTTPLYADDIADLIVYATSRKSNTVIADVLVFASNQASPYHIYRG

SEQ ID NO: 124 - protein, 269 aa, Pichia stipitis
MSFGKKAAERLANKIILITGASSGIGEATAREFASAANGNIRLILTARRKEKLAQLSDSLTKEFPTIKIHSAKLDVTEHDGIKPFISGLPKDFADIDVLINNAGKALGKASVGEISDSDIQGMMQTNVLGLINMTQAVIPIFKAKNSGDIVNIGSIAGRDPYPGGSIYCASKAAVKFFSHSLRKELINTRIRVLEVDPGAVLTEFSLVRFHGDQGAADAVYEGTQPLDASDIAEVIVFGITRKQNTVIAETLVFPSHQASASHVYKAPK

SEQ ID NO: 125 - protein, 270 aa, Debaryomyces hansenii
MSYGSKAAERVANKIVLITGASSGIGEATAKEIASAANGNLKLVLCARRKEKLDNLSKELTDKYSSIKVHVAQLDVSKLETIKPFINDLPKEFSDVDVLVNNAGLALGRDEVGTIDTDDMLSMFQTNVLGLITITQAVLPIMKKKNSGDVVNIGSIAGRDSYPGGGIYCPTKASVKSFSQVLRKELISTKIRVLEVDPGNVETEFSNVRFKGDMEKAKSVYAGTEPLLSEDVAEVVVFGLTRKQNTVIAETLVFSTNQASSSHLYRESDK

SEQ ID NO: 126 - protein, 269 aa, Pichia pastoris
MSYGLAAASRLAGKVILITGASSGIGEATALEYANAAKGEIKLALSARRFEKLEGLKEKLTTQWPNIKVHIALLDVSNIAKLTEYVESLPEEFKAVDVLVNNAGKALGIDRVGQILQEDIDGMFQTNVIGLISLTQLILPGMKARNRGDIIQLGSIAGRDPYPGGGIYCATKAAVRSFSHSLRKELIDTKIRVIEIDPGAVQTEFSLVRYKGSKESASKVYEGAEPLNALDIAELIVFASTRRENTVVAETLVFPTNQAGAGYVYRKKD

SEQ ID NO: 127 - protein, 268 aa, Candida dubliniensis
MSFGRKAAERLANRSILITGASSGIGEACAKVFAEASNGQVKLVLGARRKERLVKLSDTLIKQYPNIKIHHDFLDVTIKDSISKFIAGIPHEFEPDVLINNSGKALGKEEVGELKDEDITEMFDTNVIGVIRMTQAVLPLLKKKPYADVVFIGSIAGRVPYKNGGGYCASKAAVRSFTDTFRKETINTGIRVIEVDPGAVLTEFSVVRYKGDTDAADAVYTGTEPLTPEDVAEVVVFASSRKQNTVIADTLIFPNHQASPDHVYRKPN

SEQ ID NO: 128 - protein, 268 aa, Candida albicans
MSFGRKAAERLANRSILITGASSGIGEACAKVFAEASNGQIKLILGARRKERLIKLSDTLIKQYPNIKIHHDFLDVTIKDSISKFIAGIPTDFEPDVLINNSGKALGKEQVGELKDEDITEMLDTNVVGVIRMTQAVLPLLKKKNYADVVFIGSIAGRVAYKNGGGYCASKAAVRSFVDTFRKETIDTGIRVIEVDPGAVLTEFSVVRYKGDTEAADAVYTGTEPLTPEDVAEVVVFAASRKQNTVIADTLIFPNHQASPDHVYRKPN

SEQ ID NO: 129 - protein, 268 aa, Yarrowia lipolytica
MSFGDKAAARLAGKTVFVTGASSGIGQATVLALAEAAKGDLKFVLAARRTDRLDELKKKLETDYKGIQVLPFKLDVSKVEETENIVSKLPKEFSEVDVLINNAGMVHGTEKVGSINQNDIEIMFHTNVLGLISVTQQFVGEMRKRNKGDIVNIGSIAGREPYVGGGIYCATKAAVRSFTETLRKENIDTRIRVIEVDPGAVETEFSVVRFRGDKSKADAVYAGTEPLVADDIAEFITYTLTRRENVVIADTLIFPNHQASPTHVYRKN

SEQ ID NO: 130 - protein, 270 aa, Issatchenkia orientalis
MFGNISQRLAGKNILITGASTGIGYHTAKYFAEAANGDLKLVLAARRKEKLEALKADLLAKYPSIKVHIESLDVSKTETIAPFLKGLPEEFSIVDVLVNNAGKALGLDPIGSVDPKDVDEMFQTNVLGMIQLTQLVVQQMKERNSGDIVQLGSVAGRNPYPGGGIYCASKAALRSFTHVLREELINTKIRVIEIEPGNVATEEFSLTRFKGDKSKAEKVYEGTEPLYGTDIAELILFAVSRPQNTVIAETLVFASNQASAYHIFRGSLDK

SEQ ID NO: 131 - protein, 271 aa, Aspergillus nidulans
MASAMAKRLEGKTIVITGASSGIGRSTAREFARTAPKDLKLIVTARRIDALEELAKEIKEEVGEGVKTLPVKLDVSNPEEVKNFVPSLPAEFQDIDILVNNAGLVKGVAQAPNIDPEDINIMFATNVTGLINLTQAVLPIFKKRSDGGRGDIINIGSIAGREPYPGGSIYCSTKAAVKSFTEALRKELISTRIRVIEIDPGQVETEFSIVRFYGDKSKANAVYANCEPLTPDDIAEVIVFAAGRRENVVIADTLIFPSHQASPGHLYKKPQ

SEQ ID NO: 132 - protein, 278 aa, Aspergillus niger
MATAMAKRLEGKTILVTGASSGIGRSTAKEFARTSPKDLKIIVTARRIDSLQELAKEIKEEVGEGVKVLPVQLDVSNPEDIKKFVPSLPEEFKEIDVLVNNAGLVKGVAKAPEIAPEDINVMFSTNVTGLINMTQAILPIFKKRADGGRGDIINIGSIAGREAYPGGSIYCATKAAIRSFTDALRKELIATRIRIIEIDPGQVETEFSVVRFYGDKAKADAVYAGCEPLTPDDIAEVVVFAAGRRENVVIADTLIFPSHQVSQTSHTGLNSIADGTVT

SEQ ID NO: 133 - protein, 270 aa, Neurospora crassa
MSSAVAKRLAGKTIVITGASSGIGRSTAFEFARTAPNHGLKLILTARRVDALEQIAKEIRQEVGEGVQVLPVKLDVSQPEEVRGFVGNLPEEWRDIHVLVNNAGLVKGAPSIAEEDINVMFATNVTGLINMTQAILPIFKARGSEGGSGDIVNIGSIAGREPYAGGSIYCATKAAVRSFTDALRKELIATRIRVMEIDPGQVETEFSVVRFYGDKNKADAVYAGVDPLTPDDIAEIVVFVVTRRENVVVADTLVFPSHQAGAGIMHRKST

SEQ ID NO: 134 - protein, 259 aa, Schizosaccharomyces pombe
MSRLDGKTILITGASSGIGKSTAFEIAKVAKVKLILAARRFSTVEEIAKELESKYEVSVLPLKLDVSDLKSIPGVIESLPKEFADIDVLINNAGLALGTDKVIDLNIDDAVTMITTNVLGMMAMTRAVLPIFYSKNKGDILNVGSIAGRESYVGGSVYCSTKSALAQFTSALRKETIDTRIRIMEVDPGLVETEFSVVRFHGDKQKADNVYKNSEPLTPEDIAEVILFALTRRENVVIADTLVFPSHQGGANHVYRKQA

SEQ ID NO: 135 - protein, 268 aa, Kluyveromyces marxianus
MSQGRRAAERLQNKTIFITGASAGIGEATALEYLDAANGNVKLVLAARRLSKLQALKDKIAAEYPEAKVYIGELDVTETEKIKPFIQGLPEEFKDIDILINNAGKALGVDPVGAIDSEDIKGMIDTNVLGLINVTQAVLPIFKAKNSGDIVNLGSVAGREAYPNGSIYCATKHAVRAFTQSLRKELINTKIRVIEIAPGNVETEFSYVRYKGDTDAAKKVYKGTTPLYADDIADLIVYATSRKQNTVIADVLVFATNQASPYHIYRGE

SEQ ID NO: 136 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 137 - protein, 499 aa, Saccharomyces castellii
MTKINFEAAEPATITLPNGITYNQPTGLFINNEFMKSQDYKTIKVENPATAEIVCEVSSGTSEDVEYAVESAEHAFNDTDWATQDPKIRGRYLSKLADLMEENLELMASIETLDNGKTLALSRGDVGLAINCIRDAASYADKINGRTINSGDGYMNFTVKEPIGVCGQIIPWNFPLMMLSWKIAPALAMGNVIILKPASATPLNALFFASLCKKVGIPAGVVNIIPGPGRTVGNAITTHPRIRKAAFTGSTEIGKDIAIKASGSNLKKITLELGGKSAHLVFDDANIEKTLPNLVNGIFKNAGQICSSGSRIYVQEGIYDKLMPAFKKYVENLKVGDPFDESNFQGAITNREQYETILKYIKIGKEEGAKILTGGETIGNKGYFIKPTIFYDVKEDMEIVREEIFGPVVTVSKFKDIEDGVAMANASEFGLGAGIETENLSTALKVAKMLKSGTVWINTYNDFDSRVPFGGVKQSGYGREMGEEVYDCYTEVKAIRIKL

SEQ ID NO: 138 - protein, 497 aa, Candida glabrata
MAYKTAATKTIKLPNGLTYEQPTGLFINNEFVESSDGKTMTIENPSTQEPIVDVFSATKEDVDYAVDCAERAFEKSDWATQDPKVRARYLSKLADLMEEQLELIASIETLDNGKTIALSRGDVQLSINCIRDAASYADKVDGRSIDTGDGYMNYTIREPIGVCAQIIPWNFPLMMLSWKVGPALAMGNCIVLKPASATPLNALYFSSLCKQVGIPAGVVNIIPGPGGMVGTALTTHPKVRKVAFTGSTDIGKDIAVKASASNLKKITLELGGKSAHLVFNDANLEKTLPNLVNGIFKNAGQICSSGSRIYAQAGIYDRLLKEFKAYIEKNIKVGNPFDESNFQGAITNKEQYNTILKYINIGKEEGAKVLTGGETAAEKGYFIKPTVFYDVKEDMRVVKEEIFGPCVTISKFEEIEDGVAMANDSEFGLGAGIETENLSTALKVAKMLHSGTVWINTYNDFDSRVPFGGYKQSGYGREMGSEVYECYTQTKAVRIKL

SEQ ID NO: 139 - protein, 500 aa, Saccharomyces bayanus
MTKLHFDTAEAVKITLPNGLTYEQPTGLFINNKFTKAQDGKTYPVEDPSTENTICEVSSATTEDVEYAIECADRAFHDTEWATQDPRERGRLLSKLADELESQIDLVSSIESLDNGKTLALARGDVTIAINCLRDAAAYADKINGRTINTGDGYMNFTTLEPIGVCGQIIPWNFPIMMLTWKIAPALAMGNVCILKPAAVTPLNALYFASLCKKVGIPAGVVNIVPGPGRSVGAALTNDPRIRKLAFTGSTEVGKSVAIDSSESNLKKITLELGGKSAHLVFDDADIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVEEDMRIVKEEIFGPVVTVAKFKTLEEGVAMANSSEFGLGSGIETESLSTGLKVAKMLKAGTVWINTYNDFDSRVPFGGVKQSGYGREMGEEVYHAYTEVKAVRIKL

SEQ ID NO: 140 - protein, 507 aa, Kluyveromyces lactis
MSSTIAEKLNLKIVEQDAVSITLPNGLTYQQPTGLFINNQFIKSQDGKTLKVENPSTEEIIVEVQSATSQDVEYAVEAADAAFNSEWSTMDPKKRGSLLFKLADLIEAQKELIASIESADNGKTLALARGDVGLVIDYIRSAAGYADKLGGRTINTGDGYANFTYKEPLGVCGQIIPWNFPLMMLSWKIAPALVAGNTVILKPASPTPLNALFFASLCKEAGIPAGVVNIVPGPGRSVGDTITNHPKIRKIAFTGSTDIGRDVAIKAAQSNLKKVTLELGGKSAHLVFEDANIKKTIPNLVNGIFKNAGQICSSGSRIYVQDTIYDQLLSEFKTYLETEIKVGSPFDESNFQAAINNKAQFETILNYIDIGKKEGASILTGGERVGNKGYFIKPTVFYNVKEDMRIVKEEIFGPVVTISKFSTVDEAVALANDSEFGLGAGIETENISVALKVAKRLKAGTVWINTYNDFDAAVPFGGYKQSGYGREMGEEAFESYTQIKAVRIKLD

SEQ ID NO: 141 - protein, 526 aa, Kluyveromyces thermotolerans
MNYACLRSGSSMLGMVKAARSFSISARALSARAKADFVKITTPNGHTYEQPTGLFINNEFLKSQKGETITVEDPSTEQKIVEVQSGTKEDVEYAVECAEKAFNSSWSTGDPRNRARSLLKLADLVEERKELIASIESMDNGKTLALARGDVGIVINFLRSAAGYADKLDGRSINTGDGYVNYTIREPVGVCGQIIPWNFPLMMLSWKIAPALAAGNTVILKPASPTPLNALYFASLCKEAGIPAGVVNIIPGPGRGVGETLTTHPKIRKIAFTGSTGTGKGIAVKAAQSNLKKVTLELGGKSAHLVFNDANIEKTLPNLVNGIFLNAGQICSSGSRIYVQEGIYDKLLPAFRKYVEEKITVGSPFDENNFQGAINNKAQFETIMNYVGIGKSEGAKVLTGGEKVNDKGYFIRPTIFYDVEEDMRIVKEEVFGPVVTISKFKEIEDGVAMANDSEFGLGAGIQTENVSTALKVSKMLKAGIVWVNTYNDFDSSVPFGGCKQSGYGREMGIEAFEAYTSVKAVRIKLA

SEQ ID NO: 142 - protein, 526 aa, Kluyveromyces waltii
MNKMSLKNASTMLRMAKYARNFSVSSRILSAKMKADFVKVTTPNGLTYEQPTGLFINNEFVKSHNGATIAVEDPATEKKIVEVQSGTKEDVEYAVECAEKAFNSSWSTGDPRVRARALLKLADLIEQRKELISSIECMDNGKALFLAKNDVRIVIDYIRSSAGFADKLDGRTINTGDGYVNYTLREPVGVCGQIIPWNFPLLMLSWKIAPALAAGNTVILKPASPTPLNALYFASLCKEAGIPAGVVNIIPGPGRDVGETLTTHPKIRKIAFTGSTGTGKGIAVKAAQSNLKKVTLELGGKSAHMVFNDANLEKTIPNLVNGIFLNAGQICSSGSRIYVQEGIYDKLLPAFKKYVEEVITVGSPFDESNFQGAINNKAQFDTIMNYVGIGKEEGAKVLTGGKRSGDQGYFIRPTIFYDVNEDMRIVKEEIFGPVVTISKFKEIEDGVAMANNSEFGLGAGIQTESVSTALKVSKMLKAGTVWVNTYNDFDSSVPFGGCKQSGYGSEMGIEAFDSYTTTKAVRIKLA

SEQ ID NO: 143 - protein, 500 aa, Saccharomyces cerevisiae; identical to SEQ ID NO: 136

SEQ ID NO: 144 - protein, 500 aa, Saccharomyces cerevisiae; identical to SEQ ID NO: 136

SEQ ID NO: 145 - protein, 500 aa, Saccharomyces cerevisiae; identical to SEQ ID NO: 136

SEQ ID NO: 146 - protein, 501 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLAFKARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 147 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRTFHDTEWATQDPRERGRLLSKLADELESQIDLVSSIEALDNGKTLALARGDVTIAINCLRDAAAYADKVNGRTINTGDGYMNFTTLEPVGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 148 - protein, 500 aa, Saccharomyces cerevisiae
MTKLHFDTAEPVKITLPNGLTYEQPTGLFINNKFMKAQDGKTYPVEDPSTENTVCEVSSATPEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 149 - protein, 497 aa, Pichia pastoris
MSEFVAKINIPTIAEPVEVPTGLFINNEWVAAKSGKTFAVTSPIDESHLTDLQMAGADDVDIATDFAYKAFYKHKFVEPSVRGRWLYKLAELFEEHKDTIAKLESLDNGKALHCAQFDLNLVVEYLRSCAGYADKVDGRTINTGKDHLNFTKREPLGVCGQIIPWNFPILMWAWKIGPALATGNAVVLKPASATPLTALYATKLVKEAGIPAGLVNIVPGSGRGCGNAILQHPKIKKIAFTGSTAVGIDVMVAAATFNLKKVTLELGGKSPNIVFDDCELESTIQNLITGIFFNGGEVCCAGSRIYVQEGIYEQVLSKFKEEISKLKVGNPFEEGTYQGAQATPDQFECVLGYIERAKKAGAKLLTGGNRIGTKGYFVEPTVFYDCDEDLEIVKDEIFGPVASIGKFKDVAELVEKANNSEYGLAAGIHTQDLNKAFGVADQLEAGSVWINTYNDLHQSVPFGGYKTSGIGREMGLEAFDNYTQVKAVRTRLNVPTA

SEQ ID NO: 150 - protein, 507 aa, Kluyveromyces marxianus
MSSSLAEKLNVKIVEQKPVTVTLPNGLTYEQPTGLFINNQFIRSQDGSTLKVENPSTEEIIVEVQSATAQDVEYAVESAEAAFNSEWSSMDPRNRAAYLVKLANLIEEKKELIASIESTDNGKALALARGDVGLVIDYIRSAAGYADKLGGRTIDTGDGYANFTYREPIGVCGQIIPWNFPLMMLSWKIAPALVAGNTVILKPASPTPLNALFFASLCKEAGIPAGVVNIVPGPGRSVGDTITNHPKIRKIAFTGSTDIGRDVAIKAAQSNLKKVTLELGGKSAHLVFADANIKKTIPNLVNGIFKNAGQICSSGSRIYVQDTIYDELLSEFKKYLETEIKVGSPFDESNFQAAITNKAQFETILNYIDIGKKEGAKILTGGERVGNKGYFIRPTVFYDVKEDMRIVKEEIFGPVVTISKFSTVDEAVALANDSEFGLGAGIETENLSVALKVAKRLHAGTVWINTYNDFDAAVPFGGYKQSGYGREMGEEAFESYTQVKAVRIKLE

SEQ ID NO: 151 - protein, 503 aa, Schizosaccharomyces pombe
MSTKLVDHVEITVPTGKTYIQPVGLFINNQHVDSVHGGRVKVYSPSTEKLICEVADADEEDVDIAVKVARAAFQTDAPWRKFSSAQRGRCLSRLADCIEQNLEYLASIETLDNGKSITLARGDVQAAADCFRYYGGWADKDYGQTIETDIKRFAYTRHEPIGVCGQIIPWNFPFLMCAWKIAPAVACGNTIILKTAELTPLSALCLTKFVPECGFPPGVINVLSGDGRRCGNAISSHMDIDKVAFTGSTGVGRMVMRAAASSNLKKVTLELGGKSPNIVFNDADLDSAAVWTNYGIFYNSGQVCCAGSRVYVQEDVYDEFIKRMVAKAKTLKVGDPFAEDTFQGAQVSKQQYERIVSYIESGIAHGAKLEIGGKRHGNLGYFVEPTILSNVTEDMAVGKEEIFGPVLAVIKFKTIEEAIRRGNNSTYGLAAGVHTNNITNAIKVSNALEAGTVWVNCYNLLHHQIPFGGYKESGIGRELGSYGLTNYTQTKAVHINLGMDSPI

SEQ ID NO: 152 - protein, 496 aa, Schizosaccharomyces pombe
MSEDLFVSINFPNGKSVKQPIGLYINGEWHKSAETWETVDPSIEEVIAKVYLAGEKEIDYAVKSAKEAFKTWKKVPGSEKGELLMKLAELTEKHADTLAAIEAMDSGKPLVSNARGDVDGTIALLRYCAGWADKIYGQVIPTGPEKLAYAKRTPIGVCGQIVPWNYPLNMAGWKIAPALAAGNCIIIKSAETTPLSLLYFATLVEEAGFPKGVVNIISGLGTVAGSYMAKHPGIDKIAFTGSTKVGVIVQQLAASNLKAVTLECGGKSPFLVFEDADLDQAVKWAALGIMYNSGQICTSNSRIYVQDSVYDKFIELFKKHVIQDYIVGMPFDDNTVVGPVVNKTQYNRIKNYIEQGKKEGAKLVLGDEPLPLKQGYFISPTIFADCSENMTIVKEEIFGPVVAISKFKTEDEAIEKANNTTYGLAAMCFTKDLERAHRVSDELEAGMVFINSTENSDIQAPFGGIKMSGIGNELGSNGIEMYTQIKAVHINFNNKL

SEQ ID NO: 153 - DNA, 20 bp, Artificial Sequence
  Primer oGV770
gacgcgtgtacgcatgtaac

SEQ ID NO: 154 - DNA, 25 bp, Artificial Sequence
  Primer oGV821
cgggtaattaacgacaccctagagg

SEQ ID NO: 155 - DNA, 20 bp, Artificial Sequence
  oGV3120
gcatacaggccgcacaagag

SEQ ID NO: 156 - DNA, 20 bp, Artificial Sequence
  oGV3121
ctcattcttggctgctgttc

SEQ ID NO: 157 - DNA, 20 bp, Artificial Sequence
  oGV3703
gtccgtaccgcaaataccac

SEQ ID NO: 158 - DNA, 20 bp, Artificial Sequence
  Primer oGV3704
agaagttgttcgtgccatcc

SEQ ID NO: 159 - DNA, 20 bp, Artificial Sequence
  Primer oGV3693
aactccgcgaggtcgtccag

SEQ ID NO: 160 - DNA, 20 bp, Artificial Sequence
  Primer oGV3694
aacctgccatcacgagattt

SEQ ID NO: 161 - DNA, 60 bp, Artificial Sequence
  Primer oGV3743
gcaataagaataacaataaattcattgaacatatttcagaataacttcgtataatgtatg

SEQ ID NO: 162 - DNA, 64 bp, Artificial Sequence
  Primer oGV3744
tacaaatgagccgcgaggggccccaaatattattttgtcaataacttcgtatagcatacattat